SEQUENCE LISTING (109 sequences)

SEQ ID NO: 1 - protein, 119 aa, synthetic construct
  REGION 1..119: note = Description of sequence: Anti-HER2 D2 VH v1
EVQLVESGGGLVQPGGSLRLSCAASGFTFTDYTMDWVRQAPGKGLEWVADVNPNSGQSIYNQRFKGRFTLSVDRSKNTLYLQMNSLRAEDTAVYYCARNLGPSFYFDYWGQGTLVTVSS

SEQ ID NO: 2 - protein, 119 aa, synthetic construct
  REGION 1..119: note = Description of sequence: Anti-HER2 D2 VH v2
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPLFYFDYWGQGTLVTVSS

SEQ ID NO: 3 - protein, 119 aa, synthetic construct
  REGION 1..119: note = Description of sequence: Anti-HER2 D2 VH v3
EVQLVESGGGLVQPGGSLRLSCAASGFTFTDYTMDWVRQAPGKGLEWVADVNPNSGQSIYNQRFKGRFTLSVDRSKNTLYLQMNSLRAEDTAVYYCARNLGPLFYFDYWGQGTLVTVSS

SEQ ID NO: 4 - protein, 10 aa, synthetic construct
  REGION 1..10: note = Description of sequence: Anti-HER2 D2 CDR-H1
GFTFTDYTMD

SEQ ID NO: 5 - protein, 17 aa, synthetic construct
  REGION 1..17: note = Description of sequence: Anti-HER2D2 CDR-H2
DVNPNSGGSIYNQRFKG

SEQ ID NO: 6 - protein, 17 aa, synthetic construct
  REGION 1..17: note = Description of sequence: Anti-HER2D2 CDR-H2.1
DVNPNSGQSIYNQRFKG

SEQ ID NO: 7 - protein, 12 aa, synthetic construct
  REGION 1..12: note = Description of sequence: Anti-HER2 D2 CDR-H3
ARNLGPSFYFDY

SEQ ID NO: 8 - protein, 12 aa, synthetic construct
  REGION 1..12: note = Description of sequence: Anti-HER2 D2 CDR-H3.1
ARNLGPLFYFDY

SEQ ID NO: 9 - protein, 214 aa, synthetic construct
  REGION 1..214: note = Description of sequence: Anti-HER2 light chain v1
DIQMTQSPSSLSASVGDRVTITCRASQDVNTAVAWYQQKPGKAPKLLIYSASFLYSGVPSRFSGSRSGTDFTLTISSLQPEDFATYYCQQFYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 214 aa, synthetic construct
  REGION 1..214: note = Description of sequence: Anti-HER2 light chain v2
DIQMTQSPSSLSASVGDRVTITCRASQDVNTAVAWYQQKPGKAPKLLIYSASFLYSGVPSRFSGSRSGTDFTLTISSLQPEDFATYYCQQYYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 11 aa, synthetic construct
  REGION 1..11: note = Description of sequence: Anti-HER2 CDR-L1
RASQDVNTAVA

SEQ ID NO: 12 - protein, 7 aa, synthetic construct
  REGION 1..7: note = Description of sequence: Anti-HER2 CDR-L2
SASFLYS

SEQ ID NO: 13 - protein, 9 aa, synthetic construct
  REGION 1..9: note = Description of sequence: Anti-HER2 CDR-L3.1
QQFYTTPPT

SEQ ID NO: 14 - protein, 9 aa, synthetic construct
  REGION 1..9: note = Description of sequence: Anti-HER2 CDR-L3.2
QQYYTTPPT

SEQ ID NO: 15 - protein, 180 aa, synthetic construct
  REGION 1..180: note = Description of sequence: Anti-HER2 D4 VH
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSS

SEQ ID NO: 16 - protein, 10 aa, synthetic construct
  REGION 1..10: note = Description of sequence: Anti-HER2 D4 CDR-H1
GFNIKDTYIH

SEQ ID NO: 17 - protein, 17 aa, synthetic construct
  REGION 1..17: note = Description of sequence: Anti-HER2 D4 CDR-H2
RIYPTNGYTRYADSVKG

SEQ ID NO: 18 - protein, 12 aa, synthetic construct
  REGION 1..12: note = Description of sequence: Anti-HER2 D4 CDR-H3
RWGGDGFYAMDY

SEQ ID NO: 19 - protein, 214 aa, synthetic construct
  REGION 1..214: note = Description of sequence: Anti-HER2 light chain v3
DIQMTQSPSSLSASVGDRVTITCKASQDVSTAVAWYQQKPGKAPKLLIYSASFRYTGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 450 aa, synthetic construct
  REGION 1..450: note = Description of sequence: Anti-HER2 D4 heavy chain v1
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGEGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 450 aa, synthetic construct
  REGION 1..450: note = Description of sequence: Anti-HER2 D4 heavy chain v2
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGEGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 22 - protein, 450 aa, synthetic construct
  REGION 1..450: note = Description of sequence: Anti-HER2 D4 heavy chain v3
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGEGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 23 - protein, 450 aa, synthetic construct
  REGION 1..450: note = Description of sequence: Anti-HER2 D4 heavy chain v4
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGEGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 24 - protein, 449 aa, synthetic construct
  REGION 1..449: note = Description of sequence: Anti-HER2 D2 heavy chain v1
EVQLVESGGGLVQPGGSLRLSCAASGFTFNDYTMDWVRQAPGKGLEWVADVNPNSGGSIVNRRFKGRFTLSVDRSKNTLYLQMNSLRAEDTAVYYCARNLGPFFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 25 - protein, 449 aa, synthetic construct
  REGION 1..449: note = Description of sequence: Anti-HER2 D2 heavy chain v2
EVQLVESGGGLVQPGGSLRLSCAASGFTFTDYTMDWVRQAPGKGLEWVADVNPNSGQSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 26 - protein, 449 aa, synthetic construct
  REGION 1..449: note = Description of sequence: Anti-HER2 D2 heavy chain v3
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPLFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 27 - protein, 449 aa, synthetic construct
  REGION 1..449: note = Description of sequence: Anti-HER2 D2 heavy chain v4
EVQLVESGGGLVQPGGSLRLSCAASGFTFTDYTMDWVRQAPGKGLEWVADVNPNSGQSIYNQRFKGRFTLSVDRSKNTLYLQMNSLRAEDTAVYYCARNLGPLFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 28 - protein, 450 aa, synthetic construct
  REGION 1..450: note = Description of sequence: Anti-HER2 D4 heavy chain v5
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 29 - protein, 449 aa, synthetic construct
  REGION 1..449: note = Description of sequence: Anti-HER2 D2 heavy chain v5
EVQLVESGGGLVQPGGSLRLSCAASGFTFTDYTMDWVRQAPGKGLEWVADVNPNSGQSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 30 - protein, 449 aa, synthetic construct
  REGION 1..449: note = Description of sequence: Anti-HER2 D2 heavy chain v6
EVQLVESGGGLVQPGGSLRLSCAASGFTFTDYTMDWVRQAPGKGLEWVADVNPNSGQSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 31 - protein, 450 aa, synthetic construct
  REGION 1..450: note = Description of sequence: Anti-HER2 D4 heavy chain v6
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 32 - protein, 450 aa, synthetic construct
  REGION 1..450: note = Description of sequence: Anti-HER2 D4 heavy chain v7
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 33 - protein, 449 aa, synthetic construct
  REGION 1..449: note = Description of sequence: Anti-HER2 D2 heavy chain v7
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPLFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 34 - protein, 449 aa, synthetic construct
  REGION 1..449: note = Description of sequence: Anti-HER2 D2 heavy chain v8
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPLFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 35 - protein, 449 aa, synthetic construct
  REGION 1..449: note = Description of sequence: Anti-HER2 D2 heavy chain v9
EVQLVESGGGLVQPGGSLRLSCAASGFTFTDYTMDWVRQAPGKGLEWVADVNPNSGQSIYNQRFKGRFTLSVDRSKNTLYLQMNSLRAEDTAVYYCARNLGPLFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 36 - protein, 449 aa, synthetic construct
  REGION 1..449: note = Description of sequence: Anti-HER2 D2 heavy chain v10
EVQLVESGGGLVQPGGSLRLSCAASGFTFTDYTMDWVRQAPGKGLEWVADVNPNSGQSIYNQRFKGRFTLSVDRSKNTLYLQMNSLRAEDTAVYYCARNLGPLFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 37 - protein, 450 aa, synthetic construct
  REGION 1..450: note = Description of sequence: Anti-HER2 D4 heavy chain v8
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 38 - protein, 450 aa, synthetic construct
  REGION 1..450: note = Description of sequence: Anti-HER2 D4 heavy chain v9
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 39 - protein, 450 aa, synthetic construct
  REGION 1..450: note = Description of sequence: Anti-HER2 D4 heavy chain v10
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 40 - protein, 450 aa, synthetic construct
  REGION 1..450: note = Description of sequence: Anti-HER2 D4 heavy chain v11
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPEEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 41 - protein, 450 aa, synthetic construct
  REGION 1..450: note = Description of sequence: Anti-HER2 D4 heavy chain v12
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALSAPEEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 42 - protein, 450 aa, synthetic construct
  REGION 1..450: note = Description of sequence: Anti-HER2 D4 heavy chain v13
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 43 - protein, 449 aa, synthetic construct
  REGION 1..449: note = Description of sequence: Anti-HER2 D2 heavy chain v11
EVQLVESGGGLVQPGGSLRLSCAASGFTFTDYTMDWVRQAPGKGLEWVADVNPNSGQSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 44 - protein, 449 aa, synthetic construct
  REGION 1..449: note = Description of sequence: Anti-HER2 D2 heavy chain v12
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPLFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 45 - protein, 449 aa, synthetic construct
  REGION 1..449: note = Description of sequence: Anti-HER2 D2 heavy chain v13
EVQLVESGGGLVQPGGSLRLSCAASGFTFTDYTMDWVRQAPGKGLEWVADVNPNSGQSIYNQRFKGRFTLSVDRSKNTLYLQMNSLRAEDTAVYYCARNLGPLFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 46 - protein, 449 aa, synthetic construct
  REGION 1..449: note = Description of sequence: Anti-HER2 D2 heavy chain v14
EVQLVESGGGLVQPGGSLRLSCAASGFTFTDYTMDWVRQAPGKGLEWVADVNPNSGQSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 47 - protein, 449 aa, synthetic construct
  REGION 1..449: note = Description of sequence: Anti-HER2 D2 heavy chain v15
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPLFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 48 - protein, 449 aa, synthetic construct
  REGION 1..449: note = Description of sequence: Anti-HER2 D2 heavy chain v16
EVQLVESGGGLVQPGGSLRLSCAASGFTFTDYTMDWVRQAPGKGLEWVADVNPNSGQSIYNQRFKGRFTLSVDRSKNTLYLQMNSLRAEDTAVYYCARNLGPLFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 49 - protein, 10 aa, synthetic construct
  REGION 1..10: note = Description of sequence: Anti-HER2 CDR-H1.1
GFNFTDYTMD

SEQ ID NO: 50 - protein, 10 aa, synthetic construct
  REGION 1..10: note = Description of sequence: Anti-HER2 CDR-H1.2
GFKFTDYTMD

SEQ ID NO: 51 - protein, 10 aa, synthetic construct
  REGION 1..10: note = Description of sequence: Anti-HER2 CDR-H1.3
GFMFTDYTMD

SEQ ID NO: 52 - protein, 10 aa, synthetic construct
  REGION 1..10: note = Description of sequence: Anti-HER2 CDR-H1.4
GFHFTDYTMD

SEQ ID NO: 53 - protein, 17 aa, synthetic construct
  REGION 1..17: note = Description of sequence: Anti-HER2 CDR-H2.2
DVNPNSGGSIYNRRFKG

SEQ ID NO: 54 - protein, 17 aa, synthetic construct
  REGION 1..17: note = Description of sequence: Anti-HER2 CDR-H2.3
DVNPNSGGSIYNHRFKG

SEQ ID NO: 55 - protein, 17 aa, synthetic construct
  REGION 1..17: note = Description of sequence: Anti-HER2 CDR-H2.4
DVNPNSGGSIYNTRFKG

SEQ ID NO: 56 - protein, 12 aa, synthetic construct
  REGION 1..12: note = Description of sequence: Anti-HER2 CDR-H3.2
ARNLGPWFYFDY

SEQ ID NO: 57 - protein, 12 aa, synthetic construct
  REGION 1..12: note = Description of sequence: Anti-HER2 CDR-H3.3
ARNLGPFFYFDY

SEQ ID NO: 58 - protein, 12 aa, synthetic construct
  REGION 1..12: note = Description of sequence: Anti-HER2 CDR-H3.4
ARNLGPDFYFDY

SEQ ID NO: 59 - protein, 12 aa, synthetic construct
  REGION 1..12: note = Description of sequence: Anti-HER2 CDR-H3.5
ARNLGPYFYFDY

SEQ ID NO: 60 - protein, 119 aa, synthetic construct
  REGION 1..119: note = Description of sequence: Anti-HER2 D2 VH v4
EVQLVESGGGLVQPGGSLRLSCAASGFNFTDYTMDWVRQAPGKGLEWVADVNPNSGGSIYNQRFKGRFTLSVDRSKNTLYLQMNSLRAEDTAVYYCARNLGPSFYFDYWGQGTLVTVSS

SEQ ID NO: 61 - protein, 119 aa, synthetic construct
  REGION 1..119: note = Description of sequence: Anti-HER2 D2 VH v5
EVQLVESGGGLVQPGGSLRLSCAASGFKFTDYTMDWVRQAPGKGLEWVADVNPNSGGSIYNQRFKGRFTLSVDRSKNTLYLQMNSLRAEDTAVYYCARNLGPSFYFDYWGQGTLVTVSS

SEQ ID NO: 62 - protein, 119 aa, synthetic construct
  REGION 1..119: note = Description of sequence: Anti-HER2 D2 VH v6
EVQLVESGGGLVQPGGSLRLSCAASGFMFTDYTMDWVRQAPGKGLEWVADVNPNSGGSIYNQRFKGRFTLSVDRSKNTLYLQMNSLRAEDTAVYYCARNLGPSFYFDYWGQGTLVTVSS

SEQ ID NO: 63 - protein, 119 aa, synthetic construct
  REGION 1..119: note = Description of sequence: Anti-HER2 D2 VH v7
EVQLVESGGGLVQPGGSLRLSCAASGFHFTDYTMDWVRQAPGKGLEWVADVNPNSGGSIYNQRFKGRFTLSVDRSKNTLYLQMNSLRAEDTAVYYCARNLGPSFYFDYWGQGTLVTVSS

SEQ ID NO: 64 - protein, 119 aa, synthetic construct
  REGION 1..119: note = Description of sequence: Anti-HER2 D2 VH v8
EVQLVESGGGLVQPGGSLRLSCAASGFTFTDYTMDWVRQAPGKGLEWVADVNPNSGGSIYNRRFKGRFTLSVDRSKNTLYLQMNSLRAEDTAVYYCARNLGPSFYFDYWGQGTLVTVSS

SEQ ID NO: 65 - protein, 119 aa, synthetic construct
  REGION 1..119: note = Description of sequence: Anti-HER2 D2 VH v9
EVQLVESGGGLVQPGGSLRLSCAASGFTFTDYTMDWVRQAPGKGLEWVADVNPNSGGSIYNHRFKGRFTLSVDRSKNTLYLQMNSLRAEDTAVYYCARNLGPSFYFDYWGQGTLVTVSS

SEQ ID NO: 66 - protein, 119 aa, synthetic construct
  REGION 1..119: note = Description of sequence: Anti-HER2 D2 VH v10
EVQLVESGGGLVQPGGSLRLSCAASGFTFTDYTMDWVRQAPGKGLEWVADVNPNSGGSIYNTRFKGRFTLSVDRSKNTLYLQMNSLRAEDTAVYYCARNLGPSFYFDYWGQGTLVTVSS

SEQ ID NO: 67 - protein, 119 aa, synthetic construct
  REGION 1..119: note = Description of sequence: Anti-HER2 D2 VH v11
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPWFYFDYWGQGTLVTVSS

SEQ ID NO: 68 - protein, 119 aa, synthetic construct
  REGION 1..119: note = Description of sequence: Anti-HER2 D2 VH v12
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPFFYFDYWGQGTLVTVSS

SEQ ID NO: 69 - protein, 119 aa, synthetic construct
  REGION 1..119: note = Description of sequence: Anti-HER2 D2 VH v13
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPDFYFDYWGQGTLVTVSS

SEQ ID NO: 70 - protein, 119 aa, synthetic construct
  REGION 1..119: note = Description of sequence: Anti-HER2 D2 VH v4
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPYFYFDYWGQGTLVTVSS

SEQ ID NO: 71 - protein, 217 aa, synthetic construct
  REGION 1..217: note = Description of sequence: Fc sequence with hole mutations.hD
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 72 - protein, 217 aa, synthetic construct
  REGION 1..217: note = Description of sequence: Fc sequence with hole mutations.hE
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 73 - protein, 217 aa, synthetic construct
  REGION 1..217: note = Description of sequence: Fc sequence with hole mutations.hDE
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 74 - protein, 217 aa, synthetic construct
  REGION 1..217: note = Description of sequence: "Clone CH3C.35.23.4 with knob LALA and kD"
APEAAGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 75 - protein, 217 aa, synthetic construct
  REGION 1..217: note = Description of sequence: "Clone CH3C.35.23.4 with knob LALA and kE"
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 76 - protein, 217 aa, synthetic construct
  REGION 1..217: note = Description of sequence: "Clone CH3C.35.23.4 with knob LALA and kDE"
APEAAGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 77 - protein, 217 aa, synthetic construct
  REGION 1..217: note = Description of sequence: "Clone CH3C.35.23.4 with knob LALA and PG"
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 78 - protein, 217 aa, synthetic construct
  REGION 1..217: note = Description of sequence: "Clone CH3C.35.23.4 with knob LALA and PS"
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 79 - protein, 217 aa, synthetic construct
  REGION 1..217: note = Description of sequence: "Clone CH3C.35.23.4 with knob LALA PG and kD"
APEAAGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 80 - protein, 217 aa, synthetic construct
  REGION 1..217: note = Description of sequence: "Clone CH3C.35.23.4 with knob LALA PS and kD"
APEAAGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 81 - protein, 217 aa, synthetic construct
  REGION 1..217: note = Description of sequence: "Clone CH3C.35.23.4 with knob LALA PG and kE"
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPEEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 82 - protein, 217 aa, synthetic construct
  REGION 1..217: note = Description of sequence: "Clone CH3C.35.23.4 with knob LALA PS and kE"
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPEEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 83 - protein, 217 aa, synthetic construct
  REGION 1..217: note = Description of sequence: "Clone CH3C.35.23.4 with knob LALA PG and kDE"
APEAAGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALGAPEEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 84 - protein, 217 aa, synthetic construct
  REGION 1..217: note = Description of sequence: "Clone CH3C.35.23.4 with knob LALA PS and kDE"
APEAAGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALSAPEEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 85 - protein, 217 aa, synthetic construct
  REGION 1..217: note = Description of sequence: Fc sequence with hole mutations
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 86 - protein, 217 aa, synthetic construct
  REGION 1..217: note = Description of sequence: Clone CH3C.35.23.4 with knob and LALA mutations
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 87 - protein, 214 aa, synthetic construct
  REGION 1..214: note = Description of sequence: Anti-HER2 D4 light chain control
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 214 aa, synthetic construct
  REGION 1..214: note = Description of sequence: Anti-HER2 light chain consensus
  VAR_SEQ 91: note = X = any naturally occurring amino acid
DIQMTQSPSSLSASVGDRVTITCRASQDVNTAVAWYQQKPGKAPKLLIYSASFLYSGVPSRFSGSRSGTDFTLTISSLQPEDFATYYCQQXYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 10 aa, synthetic construct
  REGION 1..10: note = Description of sequence: Anti-HER2 CDR1 consensus
  VAR_SEQ 3: note = X = any standard amino acid, except Thr.
  VAR_SEQ 4: note = X = any standard amino acid, except Phe
  VAR_SEQ 5: note = X = any standard amino acid, except Thr
GFXXXDYTMD

SEQ ID NO: 90 - protein, 17 aa, synthetic construct
  REGION 1..17: note = Description of sequence: Anti-HER2 CDR2 consensus
  VAR_SEQ 3: note = X = any standard amino acid, except Asn
  VAR_SEQ 5: note = X = any standard amino acid, except Asn
  VAR_SEQ 6: note = X = any standard amino acid, except Ser
  VAR_SEQ 7: note = X = any standard amino acid, except Gly
  VAR_SEQ 7: note = X = any standard amino acid, except Gly
  VAR_SEQ 13: note = X = any standard amino acid, except Gln
DVXPXXXXSIYNXRFKG

SEQ ID NO: 91 - protein, 12 aa, synthetic construct
  REGION 1..12: note = Description of sequence: Anti-HER2 CDR3 consensus
  VAR_SEQ 4: note = X = any standard amino acid, except Lys
  VAR_SEQ 5: note = X = any standard amino acid, except Gly
  VAR_SEQ 6: note = X = any standard amino acid, except Pro
  VAR_SEQ 7: note = X = ny standard amino acid, except Ser
ARNXXXXFYFDY

SEQ ID NO: 92 - protein, 448 aa, synthetic construct
  REGION 1..448: note = Description of sequence: Anti-HER2 D2 heavy chain control
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 93 - protein, 449 aa, synthetic construct
  REGION 1..449: note = Description of sequence: Anti-HER2 D4 heavy chain control
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 94 - protein, 214 aa, synthetic construct
  REGION 1..214: note = Description of sequence: Anti-HER2 D2 light chain control
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 217 aa, synthetic construct
  REGION 1..217: note = Description of sequence: Wild-type human Fc sequence
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 15 aa, synthetic construct
  REGION 1..15: note = Description of sequence: Human IgG1 hinge region
EPKSCDKTHTCPPCP

SEQ ID NO: 97 - protein, 217 aa, synthetic construct
  REGION 1..217: note = Description of sequence: Clone CH3C.35.23.4
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 98 - protein, 217 aa, synthetic construct
  REGION 1..217: note = Description of sequence: Clone CH3C.35.23.4 with knob mutation
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 99 - protein, 217 aa, synthetic construct
  REGION 1..217: note = Description of sequence: Clone CH3C.35.23.4 with hole mutations
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVSKEEWQQGFVFSCSVMHEALHNHYTQKSLSLSPGK

SEQ ID NO: 100 - protein, 217 aa, synthetic construct
  REGION 1..217: note = Description of sequence: Clone CH3C.35.23.4 with hole and LALA mutations
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWSNYKTTPPVLDSDGSFFLVSKLTVSKEEWQQGFVFSCSVMHEALHNHYTQKSLSLSPGK

SEQ ID NO: 101 - protein, 217 aa, synthetic construct
  REGION 1..217: note = Description of sequence: Clone CH3C.35.23.2
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWANYKTTPPVLDSDGSFFLYSKLTVTKEEWQQGFVFSCSVMHEALHNHYTQKSLSLSPGK

SEQ ID NO: 102 - protein, 217 aa, synthetic construct
  REGION 1..217: note = Description of sequence: Clone CH3C.35.23.2 with knob mutation
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 103 - protein, 217 aa, synthetic construct
  REGION 1..217: note = Description of sequence: Clone CH3C.35.23.2 with knob and LALA mutations
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 104 - protein, 217 aa, synthetic construct
  REGION 1..217: note = Description of sequence: Clone CH3C.35.23.2 with hole mutations
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 105 - protein, 217 aa, synthetic construct
  REGION 1..217: note = Description of sequence: Clone CH3C.35.23.2 with hole and LALA mutations
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 106 - protein, 217 aa, synthetic construct
  REGION 1..217: note = Description of sequence: Clone CH3C.35.20.1
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESFGTEWSSYKTTPPVLDSDGSFFLYSKLTVTKEEWQQGFVFSCSVMHEALHNHYTQKSLSLSPGK

SEQ ID NO: 107 - protein, 217 aa, synthetic construct
  REGION 1..217: note = Description of sequence: Fc sequence with knob mutation
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 120 aa, synthetic construct
  REGION 1..120: note = Description of sequence: Anti-HER2 D4 VH v1
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGEGFYAMDYWGQGTLVTVSS

SEQ ID NO: 109 - protein, 119 aa, synthetic construct
  REGION 1..119: note = Description of sequence: Anti-HER2 D2 VH v4
EVQLVESGGGLVQPGGSLRLSCAASGFTFNDYTMDWVRQAPGKGLEWVADVNPNSGGSIVNRRFKGRFTLSVDRSKNTLYLQMNSLRAEDTAVYYCARNLGPFFYFDYWGQGTLVTVSS

(19) United States
(12) Patent Application Publication
(10) Pub. No.: US 2026/0327338 A1
(43) Pub. Date: Oct. 22, 2026

(54) ANTI-HER2 ANTIBODIES AND METHODS OF USE THEREOF

(71) Applicant: Denali Therapeutics Inc., South San Francisco, CA (US)

(72) Inventors: Abira BANDYOPADHYAY, Pleasanton, CA (US); Alisa Jayne CLEMENS, San Jose, CA (US); Do Jin KIM, Albany, NY (US); Michelle E. PIZZO, Pacifica, CA (US); Lu SHAN, Belmont, CA (US); Richard THÉOLIS, JR., Santa Cruz, CA (US); Raymond Ka Hang TONG, South San Francisco, CA (US)

(21) Appl. No.: 19/728,974

(22) Filed: Jul. 2, 2026

Related U.S. Application Data
(63) Continuation of application No. 18/685,587, filed on Aug. 25, 2022.
(60) Provisional application No. 63/237,104, filed on Aug. 25, 2021.

(51) Int. Cl. C07K 16/32; A61K 35/00; A61K 39/00; A61K 45/06

(52) U.S. Cl. CPC C07K 16/32; A61K 35/00; A61K 45/06; A61K 2039/505; C07K 2317/31; C07K 2317/526; C07K 2317/565; C07K 2317/71; C07K 2317/72; C07K 2317/732; C07K 2317/92

(57) ABSTRACT

In one aspect, antibodies that bind to subdomain II of human HER2 are provided. In another aspect, antibodies comprising a light chain polypeptide that pairs with both a heavy chain polypeptide for binding to subdomain II of human HER2 and a heavy chain polypeptide for binding to subdomain IV of human HER2 are provided. In a further aspect, antibodies that bind to both subdomain II and subdomain IV of human HER2 comprising a common light chain polypeptide are provided. Methods of treating a cancer or treating brain metastasis of a cancer using these antibodies are also provided.

### CROSS-REFERENCE TO RELATED APPLICATIONS

This application is a continuation of U.S. application Ser. No. 18/685,587, filed Feb. 22, 2024, which is a National Stage Application, filed under 35 U.S.C. § 371, of International Application No. PCT/US2022/075438, filed Aug. 25, 2022 which claims the benefit of U.S. Provisional Patent Application No. 63/237,104, filed on Aug. 25, 2021, each of which is incorporated herein by reference in its entirety for all purposes.

### SEQUENCE LISTING

The Sequence Listing written in file DNL-010-06-WO_SeqList is 168,854 bytes in size, was created Jan. 5, 2026, and is hereby incorporated by reference.

### BACKGROUND

Treatment of brain metastases of cancers such as breast cancer currently poses a daunting clinical challenge. Among breast cancer patients, the incidence of brain metastases is as high as 50%. Clinical data indicate that there is a proclivity for HER2-positive breast cancers to metastasize to the brain. Notably, anti-HER2 therapies have proven useful for the control of extracranial tumors but not intracranial lesions. The failure of these therapies to control metastatic lesions such as brain metastases of HER2-positive breast cancer is mostly attributed to an inability of the therapeutic agents to cross the blood brain barrier (BBB) and access the brain parenchyma.

### SUMMARY

In one aspect, the disclosure provides an isolated antibody comprising one or more (e.g., one, two, or all three) complementarity determining regions (CDRs) selected from the group consisting of:
(a) a heavy chain CDR1 comprising the amino acid sequence of SEQ ID NO:89;
(b) a heavy chain CDR2 comprising the amino acid sequence of SEQ ID NO:90; and
(c) a heavy chain CDR3 comprising the amino acid sequence of SEQ ID NO:91,
wherein at least one of:
   X₁ in SEQ ID NO: 89 is not T;
   X₂ in SEQ ID NO: 89 is not F;
   X₃ in SEQ ID NO: 89 is not T;
   X₁ in SEQ ID NO: 90 is not N;
   X₂ in SEQ ID NO: 90 is not N;
   X₃ in SEQ ID NO: 90 is not S;
   X₄ in SEQ ID NO: 90 is not G;
   X₅ in SEQ ID NO: 90 is not G;
   X₆ in SEQ ID NO: 90 is not Q;
   X₁ in SEQ ID NO: 91 is not L;
   X₂ in SEQ ID NO: 91 is not G;
   X₃ in SEQ ID NO: 91 is not P; and
   X₄ in SEQ ID NO: 91 is not S.

In some embodiments, the heavy chain CDR1 comprises the amino acid sequence of SEQ ID NO:89, wherein X₁ is N, K, M, or H. In some embodiments, the heavy chain CDR2 comprises the amino acid sequence of SEQ ID NO:90, wherein X₅ is Q. In some embodiments, the heavy chain CDR2 comprises the amino acid sequence of SEQ ID NO:90, wherein X₆ is R, H, or T. In some embodiments, the heavy chain CDR3 comprises the amino acid sequence of SEQ ID NO:91, wherein X₄ is W, F, D, L, or Y. In some embodiments, the heavy chain CDR3 comprises the amino acid sequence of SEQ ID NO:91, wherein X₄ is L.

In some embodiments, the antibody comprises one or more (e.g., one, two, or all three) CDRs selected from the group consisting of:
(a) a heavy chain CDR1 comprising the amino acid sequence of SEQ ID NO:89;
(b) a heavy chain CDR2 comprising the amino acid sequence of SEQ ID NO:90, wherein X₅ is Q; and
(c) a heavy chain CDR3 comprising the amino acid sequence of SEQ ID NO:91, wherein X₄ is L.

In some embodiments, the antibody comprises one or more (e.g., one, two, or all three) CDRs selected from the group consisting of:
(a) a heavy chain CDR1 having at least 90% (e.g., 91%, 92%, 93%, 94%, 95%, 96%, 97%, 98%, 99%, or 100%) sequence identity to an amino acid sequence selected from the group consisting of SEQ ID NOS:4 and 49-52 or having up to two amino acid substitutions relative to an amino acid sequence selected from the group consisting of SEQ ID NOS:4 and 49-52;
(b) a heavy chain CDR2 having at least 90% (e.g., 91%, 92%, 93%, 94%, 95%, 96%, 97%, 98%, 99%, or 100%) sequence identity to an amino acid sequence selected from the group consisting of SEQ ID NOS:5-6 and 53-55 or having up to two amino acid substitutions relative to an amino acid sequence selected from the group consisting of SEQ ID NOS:5-6 and 53-55; and
(c) a heavy chain CDR3 having at least 90% (e.g., 91%, 92%, 93%, 94%, 95%, 96%, 97%, 98%, 99%, or 100%) sequence identity to an amino acid sequence selected from the group consisting of SEQ ID NOS:7-8 and 56-59 or having up to two amino acid substitutions relative to an amino acid sequence selected from the group consisting of SEQ ID NOS:7-8 and 56-59.

In some embodiments, the antibody comprises one or more (e.g., one, two, or all three) CDRs selected from the group consisting of:
(a) a heavy chain CDR1 having at least 90% (e.g., 91%, 92%, 93%, 94%, 95%, 96%, 97%, 98%, 99%, or 100%) sequence identity to the amino acid sequence of SEQ ID NO:4 or having up to two amino acid substitutions relative to the amino acid sequence of SEQ ID NO:4;
(b) a heavy chain CDR2 having at least 90% (e.g., 91%, 92%, 93%, 94%, 95%, 96%, 97%, 98%, 99%, or 100%) sequence identity to the amino acid sequence of SEQ ID NO:5 or SEQ ID NO:6 or having up to two amino acid substitutions relative to the amino acid sequence of SEQ ID NO:5 or SEQ ID NO:6; and
(c) a heavy chain CDR3 having at least 90% (e.g., 91%, 92%, 93%, 94%, 95%, 96%, 97%, 98%, 99%, or 100%) sequence identity to the amino acid sequence of SEQ ID NO:7 or SEQ ID NO:8 or having up to two amino acid substitutions relative to the amino acid sequence of SEQ ID NO:7 or SEQ ID NO:8.

In some embodiments, the antibody comprises one or more (e.g., one, two, or all three) CDRs selected from the group consisting of:
(a) a heavy chain CDR1 comprising the amino acid sequence of SEQ ID NO:4;
(b) a heavy chain CDR2 comprising the amino acid sequence of SEQ ID NO:5 or SEQ ID NO:6; and
(c) a heavy chain CDR3 comprising the amino acid sequence of SEQ ID NO:7 or SEQ ID NO:8.

In some embodiments, the antibody comprises one or more (e.g., one, two, or all three) CDRs selected from the group consisting of:
(a) a heavy chain CDR1 comprising the amino acid sequence of SEQ ID NO:4;
(b) a heavy chain CDR2 comprising the amino acid sequence of SEQ ID NO:6; and
(c) a heavy chain CDR3 comprising the amino acid sequence of SEQ ID NO:7.

In some embodiments, the antibody comprises one or more (e.g., one, two, or all three) CDRs selected from the group consisting of:
(a) a heavy chain CDR1 comprising the amino acid sequence of SEQ ID NO:4;
(b) a heavy chain CDR2 comprising the amino acid sequence of SEQ ID NO:5; and
(c) a heavy chain CDR3 comprising the amino acid sequence of SEQ ID NO:8.

In some embodiments, the antibody comprises one or more (e.g., one, two, or all three) CDRs selected from the group consisting of:
(a) a heavy chain CDR1 comprising the amino acid sequence of SEQ ID NO:4;
(b) a heavy chain CDR2 comprising the amino acid sequence of SEQ ID NO:6; and
(c) a heavy chain CDR3 comprising the amino acid sequence of SEQ ID NO:8.

In some embodiments, the antibody comprises a heavy chain variable region comprising an amino acid sequence having at least 90% (e.g., 91%, 92%, 93%, 94%, 95%, 96%, 97%, 98%, 99%, or 100%) sequence identity to any one of SEQ ID NOS:1-3. In some embodiments, the antibody comprises a heavy chain variable region comprising the amino acid sequence of any one of SEQ ID NOS:1-3.

In a related aspect, the disclosure provides an isolated antibody heavy chain comprising one or more (e.g., one, two, or all three) of the CDRs described above. In some embodiments, the antibody heavy chain comprises a heavy chain variable region comprising an amino acid sequence having at least 90% (e.g., 91%, 92%, 93%, 94%, 95%, 96%, 97%, 98%, 99%, or 100%) sequence identity to any one of SEQ ID NOS:1-3. In some embodiments, the antibody heavy chain comprises a heavy chain variable region comprising the amino acid sequence of any one of SEQ ID NOS:1-3.

In another aspect, the disclosure provides an isolated antibody comprising:
(a) a light chain CDR3 comprising the amino acid sequence of SEQ ID NO:13 or 14.

In some embodiments, the antibody further comprises one or more (e.g., one or both) CDRs selected from the group consisting of:
(b) a light chain CDR1 having at least 90% (e.g., 91%, 92%, 93%, 94%, 95%, 96%, 97%, 98%, 99%, or 100%) sequence identity to the amino acid sequence of SEQ ID NO:11 or having up to two amino acid substitutions relative to the amino acid sequence of SEQ ID NO:11; and
(c) a light chain CDR2 having up to two amino acid substitutions relative to the amino acid sequence of SEQ ID NO:12.

In some embodiments, the antibody further comprises one or more (e.g., one or both) CDRs selected from the group consisting of:
(b) a light chain CDR1 comprising the amino acid sequence of SEQ ID NO:11; and
(c) a light chain CDR2 comprising the amino acid sequence of SEQ ID NO:12.

In some embodiments, the light chain CDR3 comprises the amino acid sequence of SEQ ID NO:13. In some embodiments, the light chain CDR3 comprises the amino acid sequence of SEQ ID NO:14.

In some embodiments, the antibody comprises a light chain variable region comprising an amino acid sequence having at least 90% (e.g., 91%, 92%, 93%, 94%, 95%, 96%, 97%, 98%, 99%, or 100%) sequence identity to any one of SEQ ID NOS:9-10. In some embodiments, the antibody comprises a light chain variable region comprising the amino acid sequence of any one of SEQ ID NOS:9-10.

In a related aspect, the disclosure provides an isolated antibody light chain comprising one or more (e.g., one, two, or all three) of the CDRs described above. In some embodiments, the antibody light chain comprises a light chain variable region comprising an amino acid sequence having at least 90% (e.g., 91%, 92%, 93%, 94%, 95%, 96%, 97%, 98%, 99%, or 100%) sequence identity to any one of SEQ ID NOS:9-10. In some embodiments, the antibody light chain comprises a light chain variable region comprising the amino acid sequence of any one of SEQ ID NOS:9-10.

In yet another aspect, the disclosure provides an isolated antibody comprising an antigen binding site comprising:
(a) a heavy chain CDR1 having at least 90% (e.g., 91%, 92%, 93%, 94%, 95%, 96%, 97%, 98%, 99%, or 100%) sequence identity to an amino acid sequence selected from the group consisting of SEQ ID NOS:4 and 49-52 or having up to two amino acid substitutions relative to an amino acid sequence selected from the group consisting of SEQ ID NOS:4 and 49-52;
(b) a heavy chain CDR2 having at least 90% (e.g., 91%, 92%, 93%, 94%, 95%, 96%, 97%, 98%, 99%, or 100%) sequence identity to an amino acid sequence selected from the group consisting of SEQ ID NOS:5-6 and 53-55 or having up to two amino acid substitutions relative to an amino acid sequence selected from the group consisting of SEQ ID NOS:5-6 and 53-55; and
(c) a heavy chain CDR3 having at least 90% (e.g., 91%, 92%, 93%, 94%, 95%, 96%, 97%, 98%, 99%, or 100%) sequence identity to an amino acid sequence selected from the group consisting of SEQ ID NOS:7-8 and 56-59 or having up to two amino acid substitutions relative to an amino acid sequence selected from the group consisting of SEQ ID NOS:7-8 and 56-59;
(d) a light chain CDR1 having at least 90% (e.g., 91%, 92%, 93%, 94%, 95%, 96%, 97%, 98%, 99%, or 100%) sequence identity to the amino acid sequence of SEQ ID NO:11 or having up to two amino acid substitutions relative to the amino acid sequence of SEQ ID NO:11;
(e) a light chain CDR2 having up to two amino acid substitutions relative to the amino acid sequence of SEQ ID NO:12; and
(f) a light chain CDR3 comprising the amino acid sequence of SEQ ID NO:13 or 14.

In some embodiments, the antigen binding site comprises:
(a) a heavy chain CDR1 having at least 90% (e.g., 91%, 92%, 93%, 94%, 95%, 96%, 97%, 98%, 99%, or 100%) sequence identity to the amino acid sequence of SEQ ID NO:4 or having up to two amino acid substitutions relative to the amino acid sequence of SEQ ID NO:4;
(b) a heavy chain CDR2 having at least 90% (e.g., 91%, 92%, 93%, 94%, 95%, 96%, 97%, 98%, 99%, or 100%) sequence identity to the amino acid sequence of SEQ ID NO:5 or SEQ ID NO:6 or having up to two amino acid substitutions relative to the amino acid sequence of SEQ ID NO:5 or SEQ ID NO:6;
(c) a heavy chain CDR3 having at least 90% (e.g., 91%, 92%, 93%, 94%, 95%, 96%, 97%, 98%, 99%, or 100%) sequence identity to the amino acid sequence of SEQ ID NO:7 or SEQ ID NO:8 or having up to two amino acid substitutions relative to the amino acid sequence of SEQ ID NO:7 or SEQ ID NO:8;
(d) a light chain CDR1 having at least 90% (e.g., 91%, 92%, 93%, 94%, 95%, 96%, 97%, 98%, 99%, or 100%) sequence identity to the amino acid sequence of SEQ ID NO:11 or having up to two amino acid substitutions relative to the amino acid sequence of SEQ ID NO:11;
(e) a light chain CDR2 having up to two amino acid substitutions relative to the amino acid sequence of SEQ ID NO:12; and
(f) a light chain CDR3 comprising the amino acid sequence of SEQ ID NO:13 or 14.

In some embodiments, the antigen binding site comprises a heavy chain variable region comprising an amino acid sequence having at least 90% (e.g., 91%, 92%, 93%, 94%, 95%, 96%, 97%, 98%, 99%, or 100%) sequence identity to any one of SEQ ID NOS:1-3 and a light chain variable region comprising an amino acid sequence having at least 90% (e.g., 91%, 92%, 93%, 94%, 95%, 96%, 97%, 98%, 99%, or 100%) sequence identity to any one of SEQ ID NOS:9-10. In some embodiments, the antigen binding site comprises a heavy chain variable region comprising the amino acid sequence of any one of SEQ ID NOS:1-3 and a light chain variable region comprising the amino acid sequence of any one of SEQ ID NOS:9-10.

In some embodiments, the antibody further comprises a second antigen binding site comprising one or more CDRs selected from the group consisting of:
(a) a heavy chain CDR1 comprising the amino acid sequence of SEQ ID NO:16 or having up to two amino acid substitutions relative to the amino acid sequence of SEQ ID NO:16;
(b) a heavy chain CDR2 having at least 90% (e.g., 91%, 92%, 93%, 94%, 95%, 96%, 97%, 98%, 99%, or 100%) sequence identity to the amino acid sequence of SEQ ID NO:17 or having up to two amino acid substitutions relative to the amino acid sequence of SEQ ID NO:17; and
(c) a heavy chain CDR3 having at least 90% (e.g., 91%, 92%, 93%, 94%, 95%, 96%, 97%, 98%, 99%, or 100%) sequence identity to the amino acid sequence of SEQ ID NO:18 or having up to two amino acid substitutions relative to the amino acid sequence of SEQ ID NO:18.

In some embodiments, the second antigen binding site comprises a heavy chain variable region comprising an amino acid sequence having at least 90% (e.g., 91%, 92%, 93%, 94%, 95%, 96%, 97%, 98%, 99%, or 100%) sequence identity to SEQ ID NO:15. In some embodiments, the second antigen binding site comprises a heavy chain variable region comprising the sequence of SEQ ID NO:15.

In some embodiments, the second antigen binding site further comprises one or more CDRs selected from the group consisting of:
(a) a light chain CDR1 having at least 90% (e.g., 91%, 92%, 93%, 94%, 95%, 96%, 97%, 98%, 99%, or 100%) sequence identity to the amino acid sequence of SEQ ID NO:11 or having up to two amino acid substitutions relative to the amino acid sequence of SEQ ID NO:11;
(b) a light chain CDR2 having up to two amino acid substitutions relative to the amino acid sequence of SEQ ID NO:12; and
(c) a light chain CDR3 having up to two amino acid substitutions relative to the amino acid sequence of SEQ ID NO:13 or 14.

In some embodiments, the second antigen binding site comprises a light chain variable region comprising an amino acid sequence having at least 90% (e.g., 91%, 92%, 93%, 94%, 95%, 96%, 97%, 98%, 99%, or 100%) sequence identity to any one of SEQ ID NOS:9-10. In some embodiments, the second antigen binding site comprises a light chain variable region comprising the sequence of any one of SEQ ID NOS:9-10.

In some embodiments, the first and second antigen binding sites comprise the same light chain CDR1, CDR2, and CDR3 sequences. In some embodiments, the antibody comprises heavy and light chain CDRs selected from the combinations listed in Table 1.

In a related aspect, the disclosure provides an isolated antibody comprising heavy and light chains selected from the combinations listed in Table 2.

In a further aspect, the disclosure provides an isolated antibody comprising:
(a) a first antigen binding site for human epidermal growth factor receptor 2 (HER2) subdomain IV;
(b) a second antigen binding site for human HER2 subdomain II; and
(c) a modified Fc polypeptide dimer comprising a first Fc polypeptide that contains modifications that create a TfR-binding site,
wherein a light chain polypeptide sequence in the first antigen binding site is identical to a light chain polypeptide sequence in the second antigen binding site.

In a related aspect, the disclosure provides an isolated antibody comprising:
(a) a first antigen binding site for human HER2 subdomain II;
(b) a second antigen binding site for human HER2 subdomain IV; and
(c) a modified Fc polypeptide dimer comprising a first Fc polypeptide that contains modifications that create a TfR-binding site,
wherein a light chain polypeptide sequence in the first antigen binding site is identical to a light chain polypeptide sequence in the second antigen binding site.

In some embodiments, the first Fc polypeptide comprises a modified CH3 domain comprising the TfR-binding site. In some embodiments, the modified CH3 domain is derived from a human IgG1, IgG2, IgG3, or IgG4 CH3 domain.

In some embodiments, the modified CH3 domain comprises one, two, three, four, five, six, seven, eight, nine, ten, or eleven substitutions in a set of amino acid positions comprising 380, 384, 386, 387, 388, 389, 390, 413, 415, 416, and 421, according to EU numbering. In some embodiments, the modified CH3 domain comprises Glu, Leu, Ser, Val, Trp, Tyr, or Gln at position 380; Leu, Tyr, Phe, Trp, Met, Pro, or Val at position 384; Leu, Thr, His, Pro, Asn, Val, or Phe at position 386; Val, Pro, Ile, or an acidic amino acid at position 387; Trp at position 388; an aliphatic amino acid, Gly, Ser, Thr, or Asn at position 389; Gly, His, Gln, Leu, Lys, Val, Phe, Ser, Ala, Asp, Glu, Asn, Arg, or Thr at position 390; an acidic amino acid, Ala, Ser, Leu, Thr, Pro, Ile, or His at position 413; Glu, Ser, Asp, Gly, Thr, Pro, Gln, or Arg at position 415; Thr, Arg, Asn, or an acidic amino acid at position 416; and/or an aromatic amino acid, His, or Lys at position 421, according to EU numbering.

In some embodiments, the first Fc polypeptide that contains modifications that create the TfR-binding site binds to the apical domain of TfR.

In some embodiments, the first Fc polypeptide and the second Fc polypeptide each comprises modifications that promote heterodimerization. In some embodiments, the first Fc polypeptide comprises a T366W substitution and the second Fc polypeptide comprises T366S, L368A, and Y407V substitutions, according to EU numbering. In other embodiments, the first Fc polypeptide comprises T366S, L368A, and Y407V substitutions and the second Fc polypeptide comprises a T366W substitution, according to EU numbering.

In some embodiments, the first Fc polypeptide and/or the second Fc polypeptide independently comprises modifications that reduce TfR-mediated effector function. In some embodiments, the modifications that reduce effector function are L234A and L235A substitutions, according to EU numbering. In certain embodiments, the first Fc polypeptide specifically binds to TfR and comprises L234A and L235A substitutions. In certain embodiments, the first Fc polypeptide further comprises a P329G or a P329S substitution, according to EU numbering. In certain embodiments, the second Fc polypeptide comprises Leu at positions 234 and 235 and a proline at position 329, according to EU numbering. In other embodiments, the second Fc polypeptide specifically binds to TfR and comprises L234A and L235A substitutions. In certain embodiments, the second Fc polypeptide further comprises a P329G or a P329S substitution, according to EU numbering. In certain embodiments, the first Fc polypeptide comprises Leu at positions 234 and 235 and a proline at position 329, according to EU numbering.

In some embodiments, a hinge region or a portion thereof is linked to the N-terminus of the first Fc polypeptide and/or the second Fc polypeptide.

In some embodiments, the first Fc polypeptide and/or the second Fc polypeptide independently comprises a sequence having at least 90% (e.g., 91%, 92%, 93%, 94%, 95%, 96%, 97%, 98%, 99%, or 100%) identity to a sequence selected from the group consisting of SEQ ID NOS:71-86 and 98-100. In some embodiments, the first Fc polypeptide or the second Fc polypeptide comprises a sequence having at least 90% (e.g., 91%, 92%, 93%, 94%, 95%, 96%, 97%, 98%, 99%, or 100%) identity to a sequence selected from the group consisting of SEQ ID NOS:71-73, 85, and 99-100. In other embodiments, the first Fc polypeptide or the second Fc polypeptide comprises a sequence having at least 90% (e.g., 91%, 92%, 93%, 94%, 95%, 96%, 97%, 98%, 99%, or 100%) identity to a sequence selected from the group consisting of SEQ ID NOS:74-84, 86 and 98.

In some embodiments of this antibody, the first antigen binding site comprises the amino acid sequence of SEQ ID NO:15; the second antigen binding site comprises an amino acid sequence selected from the group consisting of SEQ ID NOS:1-3 and 60-70; the first Fc polypeptide that contains modifications that create the TfR-binding site comprises an amino acid sequence selected from the group consisting of SEQ ID NOS:74-84, 86, and 98; and the light chain polypeptide sequence comprises the amino acid sequence of SEQ ID NO:9 or SEQ ID NO:10. In some embodiments, the antibody further comprises a second Fc polypeptide comprising an amino acid sequence selected from the group consisting of SEQ ID NOS:71-73, 85, and 99-100.

In other embodiments of this antibody, the first antigen binding site comprises an amino acid sequence selected from the group consisting of SEQ ID NOS:1-3 and 60-70; the second antigen binding site comprises the amino acid sequence of SEQ ID NO:15; the first Fc polypeptide that contains modifications that create the TfR-binding site comprises an amino acid sequence selected from the group consisting of SEQ ID NOS:74-84, 86, and 98; and the light chain polypeptide sequence comprises the amino acid sequence of SEQ ID NO:9 or SEQ ID NO:10. In some embodiments, the antibody further comprises a second Fc polypeptide comprising an amino acid sequence selected from the group consisting of SEQ ID NOS:71-73, 85, and 99-100.

In some embodiments, the first Fc polypeptide and/or the second Fc polypeptide independently comprises a S239D and/or a I332E substitution, according to EU numbering. In some embodiments, the first Fc polypeptide and/or the second Fc polypeptide independently comprising the S239D and/or the I332E substitution is capable of enhancing HER2-mediated effector function.

In some embodiments of this antibody:
(a) the first Fc polypeptide comprises a S239D substitution and the second Fc polypeptide comprises a S239D substitution, according to EU numbering;
(b) the first Fc polypeptide comprises a I332E substitution and the second Fc polypeptide comprises a S239D substitution, according to EU numbering;
(c) the first Fc polypeptide comprises a S239D and a I332E substitution and the second Fc polypeptide comprises a S239D substitution, according to EU numbering;
(d) the second Fc polypeptide comprises a S239D substitution, according to EU numbering;
(e) the first Fc polypeptide comprises a S239D substitution and the second Fc polypeptide comprises a I332E substitution, according to EU numbering;
(f) the first Fc polypeptide comprises a I332E substitution and the second Fc polypeptide comprises a I332E substitution, according to EU numbering;
(g) the first Fc polypeptide comprises a S239D and a I332E substitution and the second Fc polypeptide comprises a I332E substitution, according to EU numbering;
(h) the second Fc polypeptide comprises a I332E substitution, according to EU numbering;
(i) the first Fc polypeptide comprises a S239D substitution and the second Fc polypeptide comprises a S239D and a I332E substitution, according to EU numbering;
(j) the first Fc polypeptide comprises a I332E substitution and the second Fc polypeptide comprises a S239D and a I332E substitution, according to EU numbering;
(k) the first Fc polypeptide comprises a S239D and a I332E substitution and the second Fc polypeptide comprises a S239D and a I332E substitution, according to EU numbering;
(l) the second Fc polypeptide comprises a S239D and a I332E substitution, according to EU numbering;
(m) the first Fc polypeptide comprises a S239D substitution, according to EU numbering;
(n) the first Fc polypeptide comprises a I332E substitution, according to EU numbering; or
(o) the first Fc polypeptide comprises a S239D and a I332E substitution, according to EU numbering.

In certain embodiments of this antibody:
(a) the first Fc polypeptide comprises a I332E substitution and the second Fc polypeptide comprises a S239D substitution, according to EU numbering;
(b) the first Fc polypeptide comprises a S239D and a I332E substitution and the second Fc polypeptide comprises a S239D substitution, according to EU numbering;
(c) the first Fc polypeptide comprises a S239D substitution and the second Fc polypeptide comprises a I332E substitution, according to EU numbering;
(d) the second Fc polypeptide comprises a I332E substitution, according to EU numbering;
(e) the first Fc polypeptide comprises a S239D substitution and the second Fc polypeptide comprises a S239D and a I332E substitution, according to EU numbering; or
(f) the first Fc polypeptide comprises a I332E substitution, according to EU numbering.

In particular embodiments of this antibody:
(a) the first Fc polypeptide comprises a I332E substitution and a serine at position 239, and the second Fc polypeptide comprises a S239D substitution and a isoleucine at position 332, according to EU numbering;
(b) the first Fc polypeptide comprises a S239D and a I332E substitution, and the second Fc polypeptide comprises a S239D substitution and a isoleucine at position 332, according to EU numbering;
(c) the first Fc polypeptide comprises a S239D substitution and a isoleucine at position 332, and the second Fc polypeptide comprises a I332E substitution and a serine at position 239, according to EU numbering;
(d) the first Fc polypeptide comprises a serine at position 239 and a isoleucine at 332, and the second Fc polypeptide comprises a I332E substitution and a serine at position 239, according to EU numbering;
(e) the first Fc polypeptide comprises a S239D substitution and a isoleucine at position 332, and the second Fc polypeptide comprises a S239D and a I332E substitution, according to EU numbering; or
(f) the first Fc polypeptide comprises a I332E substitution and a serine at position 239, according to EU numbering, and the second Fc polypeptide comprises a serine at position 239 and a isoleucine at 332.

In some embodiments, the antibody comprises two heavy chains and two light chains. In certain embodiments, the antibody comprises heavy and light chains selected from the combinations listed in Table 2. In certain embodiments, the first heavy chain comprises a V_{H} and a Fc sequence selected from the combinations in Table 3 and the second heavy chain comprises a V_{H} and a Fc sequence selected from the combinations in Table 4. In certain embodiments, the first heavy chain comprises a V_{H} and a Fc sequence selected from the combinations in Table 5 and the second heavy chain comprises a V_{H} and a Fc sequence selected from the combinations in Table 6.

In another aspect, the disclosure provides a pharmaceutical composition comprising any of the antibodies described herein and a pharmaceutically acceptable carrier.

In another aspect, the disclosure provides an isolated polynucleotide comprising a nucleotide sequence encoding an antibody described herein.

In another aspect, the disclosure provides a vector comprising the polynucleotide of the previous aspect.

In another aspect, the disclosure provides a host cell comprising the polynucleotide or the vector.

In another aspect, the disclosure provides a method for treating a cancer or treating brain metastasis of a cancer in a subject, the method comprising administering to the subject a therapeutically effective amount of an antibody described herein or a pharmaceutical composition thereof.

In some embodiments, the antibody is adminstered in combination with a chemotherapy or radiation therapy. In some embodiments, the cancer is a metastatic cancer. In some embodiments, the cancer is a breast cancer. In some embodiments, the cancer is a HER2-positive cancer.

### BRIEF DESCRIPTION OF THE DRAWINGS

FIG. 1 is a schematic drawing showing an exemplary bispecific antibody having a first antigen binding site for human HER2 subdomain IV (“Anti-HER2_D4”) and a second antigen binding site for human HER2 subdomain II (“Anti-HER2_D2”), in which the first and second antigen binding sites include the identical light chain polypeptide, and an Fc polypeptide dimer comprising a first Fc polypeptide having a TfR-binding site and a knob mutation and a second Fc polypeptide having a hole mutation.

FIG. 2 shows growth inhibition assay results on ZR-75-30 cells as well as IC50 and max % growth inhibition values for the different antibodies in Table 12.

FIGS. 3A and 3B illustrate in vivo anti-tumor activity tumor in single dose study with ATV:CLC bispecific antibody in 2 human cell line derived xenograft models. FIG. 3A: BT-474; FIG. 3B: OE19.

FIGS. 4A and 4B illustrate in vivo anti-tumor activity tumor in single dose lower dose study with ATV:CLC bispecific antibody in 2 human cell line derived xenograft models. FIG. 4A: BT-474; FIG. 4B: OE19.

FIGS. 5A and 5B illustrate in vivo anti-tumor activity tumor in multidose study with ATV:CLC bispecific antibody in 2 human cell line derived xenograft models. FIG. 5A: BT-474; FIG. 5B: OE19.

FIG. 6 illustrates brain uptake of ATV:CLC bispecific antibody.

FIGS. 7A and 7B illustrate IHC brain distribution of CLC bispecific antibodies.

FIG. 8 illustrates plasma PK in single dose study with ATV:CLC bispecific antibodies in cynomolgus monkeys.

FIG. 9 illustrates ADCC of ATV:CLC bispecific antibodies.

### DETAILED DESCRIPTION

### I. Introduction

Described herein are anti-HER2 bispecific antibodies that utilize a common light chain approach, i.e., two antigen binding domains that are paired with an identical light chain but still retain separate specificities. The use of a common light chain prevents light chain mispairing and as a result makes it easier to manufacture these bispecific antibodies. In some embodiments, the bispecific antibodies comprise a first antigen binding site for human HER2 subdomain IV and a second antigen binding site for human HER2 subdomain II, wherein the light chain polypeptide sequence in the first antigen binding site is identical to the light chain polypeptide sequence in the second antigen binding site. In another embodiment, the bispecific antibodies comprise a first antigen binding site for human HER2 subdomain II and a second antigen binding site for human HER2 subdomain IV, wherein the light chain polypeptide sequence in the first antigen binding site is identical to the light chain polypeptide sequence in the second antigen binding site.

Furthermore, previous therapies have failed to control brain metastases of HER2-positive breast cancer mostly because of the inability of the therapeutic agents to cross the blood brain barrier (BBB) and access the brain parenchyma. Thus, there is a need for new therapeutic agents that can cross the BBB and target HER2 in the brain parenchyma. We previously described the use of transferrin receptor (TfR)-binding as a method to enable BBB delivery across the brain endothelium, as the expression of TfR is highly expressed in brain endothelial cells and can enable BBB delivery via receptor-mediated transcytosis. Interestingly, TfR is highly expressed in various cancers, including HER2-positive breast cancers. The mechanism by which cancer cells acquire increased TfR expression likely relates to tumor cell proliferation and increased metabolic demand such as iron uptake. In fact, public microarray datasets demonstrated a correlation of TfR expression to breast cancer prognosis (Miller et al., Cancer Res. 71:6728, 2011). There have also been some reports on the use of TfR as a pharmacological target for various types of cancers.

In some embodiments, the anti-HER2 bispecific antibody comprises one or more modified Fc polypeptides that specifically bind to a BBB receptor, e.g., TfR (i.e., TfR-binding Fc polypeptides). In some embodiments, the anti-HER2 bispecific antibody is capable of being transported across the BBB. In some embodiments, the anti-HER2 bispecific antibodies binding to both HER2 and TfR as described herein can provide additional anti-tumor benefits upon binding to HER2-positive tumor cells which also express high levels of TfR, compared to other therapeutic agents that bind to HER2 alone. Specifically, since these antibodies can bind both the TfR and HER2 at the same time, this could enhance their potency and/or efficacy.

### II. Definitions

As used herein, the singular forms “a,” “an,” and “the” include plural referents unless the content clearly dictates otherwise. Thus, for example, reference to “an antibody” optionally includes a combination of two or more such molecules, and the like.

As used herein, the terms “about” and “approximately,” when used to modify an amount specified in a numeric value or range indicate that the numeric value as well as reasonable deviations from the value known to the skilled person in the art, for example ±20%, ±10%, or ±5%, are within the intended meaning of the recited value.

The terms “human epidermal growth factor receptor 2,” “HER2,” “HER2/neu,” and “ERBB2” (also known as CD340, receptor tyrosine-protein kinase erbB-2, proto-oncogene and Neu,) refer to a tyrosine receptor kinase protein encoded by the ERBB2 gene in humans that is a member of the human epidermal growth factor receptor (HER/EGFR/ERBB) family. Amplification or overexpression of HER2 plays a significant role in the development and progression of certain aggressive types of cancer, including breast cancer. Non-limiting examples of human HER2 nucleotide sequences are set forth in GenBank reference numbers NP 001005862, NP_001289936, NP_001289937, NP_001289938, and NP_004448. Non-limiting examples of human HER2 peptide sequences are set forth in GenBank reference numbers NP_001005862, NP_001276865, NP_001276866, NP_001276867, and NP_004439.

The extracellular domain of HER2, which contains approximately 600 amino acids, includes four subdomains (subdomains I, II, III, and IV). Subdomains I and III form a ligand binding site. The cysteine-rich subdomains II and IV are involved in receptor homodimerization and heterodimerization. Anti-HER2 antibodies can bind to specific subdomains (e.g., subdomain II and/or subdomain IV).

As used herein, the term “anti-HER2_D2” or “anti-HER2_D4” refers to an antibody that binds to subdomain II or IV, respectively, of human HER2.

As used herein, the term “antibody” refers to a protein with an immunoglobulin fold that specifically binds to an antigen via its variable regions. The term encompasses intact polyclonal antibodies, intact monoclonal antibodies, single chain antibodies, multispecific antibodies such as bispecific antibodies, monospecific antibodies, monovalent antibodies, chimeric antibodies, humanized antibodies, and human antibodies. The term “antibody,” as used herein, also includes antibody fragments that retain antigen-binding specificity, including but not limited to Fab, F(ab′)₂, Fv, scFv, and bivalent scFv. Antibodies can contain light chains that are classified as either kappa or lambda. Antibodies can contain heavy chains that are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain” (V_{L}) and “variable heavy chain” (V_{H}) refer to these light and heavy chains, respectively.

The term “variable region” or “variable domain” refers to a domain in an antibody heavy chain or light chain that is derived from a germline Variable (V) gene, Diversity (D) gene, or Joining (J) gene (and not derived from a Constant (Cμ and Cδ) gene segment), and that gives an antibody its specificity for binding to an antigen. Typically, an antibody variable region comprises four conserved “framework” regions interspersed with three hypervariable “complementarity determining regions.”

The term “complementarity determining region” or “CDR” refers to the three hypervariable regions in each chain that interrupt the four framework regions established by the light and heavy chain variable regions. The CDRs are primarily responsible for antibody binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 or CDR-H3 is located in the variable region of the heavy chain of the antibody in which it is found, whereas a V_{L} CDR1 or CDR-L1 is the CDR1 from the variable region of the light chain of the antibody in which it is found.

The “framework regions” or “FRs” of different light or heavy chains are relatively conserved within a species. The framework region of an antibody, that is the combined framework regions of the constituent light and heavy chains, serves to position and align the CDRs in three-dimensional space. Framework sequences can be obtained from public DNA databases or published references that include germline antibody gene sequences. For example, germline DNA sequences for human heavy and light chain variable region genes can be found in the “VBASE2” germline variable gene sequence database for human and mouse sequences.

The amino acid sequences of the CDRs and framework regions can be determined using various well known definitions in the art, e.g., Kabat, Chothia, international ImMunoGeneTics database (IMGT), AbM, and observed antigen contacts (“Contact”). In some embodiments, CDRs are determined according to the Contact definition. See, MacCallum et al., J. Mol. Biol. 262:732-745, 1996. In some embodiments, CDRs are determined by a combination of Kabat, Chothia, and/or Contact CDR definitions.

The term “epitope” refers to the area or region of an antigen to which a molecule, e.g., the CDRs of an antibody, specifically binds and can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids (e.g., a linear epitope), or amino acids from different parts of the protein that are brought into proximity by protein folding (e.g., a discontinuous or conformational epitope).

As used herein, the phrase “recognizes an epitope,” as used with reference to an antibody, means that the antibody CDRs interact with or specifically bind to the antigen at that epitope or a portion of the antigen containing that epitope.

A “humanized antibody” is a chimeric immunoglobulin derived from a non-human source (e.g., murine) that contains minimal sequences derived from the non-human immunoglobulin outside the CDRs. In general, a humanized antibody will comprise at least one (e.g., two) variable domain(s), in which the CDR regions substantially correspond to those of the non-human immunoglobulin and the framework regions substantially correspond to those of a human immunoglobulin sequence. In some instances, certain framework region residues of a human immunoglobulin can be replaced with the corresponding residues from a non-human species to, e.g., improve specificity, affinity, and/or serum half-life. The humanized antibody can also comprise at least a portion of an immunoglobulin constant region (Fc), typically that of a human immunoglobulin sequence. Methods of antibody humanization are known in the art.

A “human antibody” or a “fully human antibody” is an antibody having human heavy chain and light chain sequences, typically derived from human germline genes. In some embodiments, the antibody is produced by a human cell, by a non-human animal that utilizes human antibody repertoires (e.g., transgenic mice that are genetically engineered to express human antibody sequences), or by phage display platforms.

The term “specifically binds” refers to a molecule, e.g., an antibody as described herein, that binds to an epitope or target with greater affinity, greater avidity, and/or greater duration to that epitope or target in a sample than it binds to another epitope or non-target compound (e.g., a structurally different antigen). In some embodiments, a molecule that specifically binds to an epitope or target is a molecule that binds to the epitope or target with at least 5-fold greater affinity than other epitopes or non-target compounds, e.g., at least 6-fold, 7-fold, 8-fold, 9-fold, 10-fold, 25-fold, 50-fold, 100-fold, 1000-fold, 10,000-fold, or greater affinity. The term “specific binding,” “specifically binds to,” or “is specific for” a particular epitope or target, as used herein, can be exhibited, for example, by a molecule having an equilibrium dissociation constant K_{D} for the epitope or target to which it binds of, e.g., 10⁻⁴ M or smaller, e.g., 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. It will be recognized by one of skill that a molecule that specifically binds to a target from one species may also specifically bind to orthologs of that target.

The term “binding affinity” is used herein to refer to the strength of a non-covalent interaction between two molecules, e.g., between an antibody as described herein and an antigen. Thus, for example, the term may refer to 1:1 interactions between an antibody and an antigen, unless otherwise indicated or clear from context. Binding affinity may be quantified by measuring an equilibrium dissociation constant (K_{D}), which refers to the dissociation rate constant (k_{d}, time⁻¹) divided by the association rate constant (kₐ, time⁻¹ M⁻¹). K_{D} can be determined by measurement of the kinetics of complex formation and dissociation, e.g., using Surface Plasmon Resonance (SPR) methods, e.g., a Biacore™ system; kinetic exclusion assays such as KinExA®, and BioLayer interferometry (e.g., using the ForteBio® Octet platform). As used herein, “binding affinity” includes not only formal binding affinities, such as those reflecting 1:1 interactions between an antibody and an antigen, but also apparent affinities for which K_{D}'s are calculated that may reflect avid binding.

A “transferrin receptor” or “TfR,” as used herein, refers to transferrin receptor protein 1. The human transferrin receptor 1 polypeptide sequence is set forth in SEQ ID NO:150. Transferrin receptor protein 1 sequences from other species are also known (e.g., chimpanzee, accession number XP_003310238.1; rhesus monkey, NP 001244232.1; dog, NP_001003111.1; cattle, NP_001193506.1; mouse, NP_035768.1; rat, NP_073203.1; and chicken, NP_990587.1). The term “transferrin receptor” also encompasses allelic variants of exemplary reference sequences, e.g., human sequences, that are encoded by a gene at a transferrin receptor protein 1 chromosomal locus. Full-length transferrin receptor protein includes a short N-terminal intracellular region, a transmembrane region, and a large extracellular domain. The extracellular domain is characterized by three domains: a protease-like domain, a helical domain, and an apical domain.

As used herein, the term “Fc polypeptide” refers to the C-terminal region of a naturally occurring immunoglobulin heavy chain polypeptide that is characterized by an Ig fold as a structural domain. An Fc polypeptide contains constant region sequences including at least the CH2 domain and/or the CH3 domain and may contain at least part of the hinge region, but does not contain a variable region.

A “modified Fc polypeptide” refers to an Fc polypeptide that has at least one mutation, e.g., a substitution, deletion or insertion, as compared to a wild-type immunoglobulin heavy chain Fc polypeptide sequence, but retains the overall Ig fold or structure of the native Fc polypeptide.

As used herein, “FcRn” refers to the neonatal Fc receptor. Binding of Fc polypeptides to FcRn reduces clearance and increases serum half-life of the Fc polypeptide. The human FcRn protein is a heterodimer that is composed of a protein of about 50 kDa in size that is similar to a major histocompatibility (MHC) class I protein and a β2-microglobulin of about 15 kDa in size.

As used herein, an “FcRn binding site” refers to the region of an Fc polypeptide that binds to FcRn. In human IgG, the FcRn binding site, as numbered using the EU index, includes L251, M252, I253, S254, R255, T256, M428, H433, N434, H435, and Y436. These positions correspond to positions 21 to 26, 198, and 203 to 206 of SEQ ID NO:95.

As used herein, a “native FcRn binding site” refers to a region of an Fc polypeptide that binds to FcRn and that has the same amino acid sequence as the region of a naturally occurring Fc polypeptide that binds to FcRn.

As used herein, the terms “CH3 domain” and “CH2 domain” refer to immunoglobulin constant region domain polypeptides. For purposes of this application, a CH3 domain polypeptide refers to the segment of amino acids from about position 341 to about position 447 as numbered according to the EU numbering scheme, and a CH2 domain polypeptide refers to the segment of amino acids from about position 231 to about position 340 as numbered according to the EU numbering scheme and does not include hinge region sequences. CH2 and CH3 domain polypeptides may also be numbered by the IMGT (ImMunoGeneTics) numbering scheme in which the CH2 domain numbering is 1-110 and the CH3 domain numbering is 1-107, according to the IMGT Scientific chart numbering (IMGT website). CH2 and CH3 domains are part of the Fc region of an immunoglobulin. An Fc region refers to the segment of amino acids from about position 231 to about position 447 as numbered according to the EU numbering scheme, but as used herein, can include at least a part of a hinge region of an antibody. An illustrative hinge region sequence is the human IgG1 hinge sequence EPKSCDKTHTCPPCP (SEQ ID NO:96).

The terms “wild-type,” “native,” and “naturally occurring,” as used with reference to a CH3 or CH2 domain, refer to a domain that has a sequence that occurs in nature.

As used herein, the term “mutant,” as used with reference to a mutant polypeptide or mutant polynucleotide is used interchangeably with “variant.” A variant with respect to a given wild-type CH3 or CH2 domain reference sequence can include naturally occurring allelic variants. A “non-naturally” occurring CH3 or CH2 domain refers to a variant or mutant domain that is not present in a cell in nature and that is produced by genetic modification, e.g., using genetic engineering technology or mutagenesis techniques, of a native CH3 domain or CH2 domain polynucleotide or polypeptide. A “variant” includes any domain comprising at least one amino acid mutation with respect to wild-type. Mutations may include substitutions, insertions, and deletions.

The term “isolated,” as used with reference to a nucleic acid or protein, denotes that the nucleic acid or protein is essentially free of other cellular components with which it is associated in the natural state. It is preferably in a homogeneous state. Purity and homogeneity are typically determined using analytical chemistry techniques such as electrophoresis (e.g., polyacrylamide gel electrophoresis) or chromatography (e.g., high performance liquid chromatography). In some embodiments, an isolated nucleic acid or protein is at least 85% pure, at least 90% pure, at least 95% pure, or at least 99% pure.

The term “amino acid” refers to naturally occurring and synthetic amino acids, as well as amino acid analogs and amino acid mimetics that function in a manner similar to the naturally occurring amino acids. Naturally occurring are those encoded by the genetic code, as well as those amino acids that are later modified, e.g., hydroxyproline, γ-carboxyglutamate and O-phosphoserine. Naturally occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof. “Amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids.

The term “protein” as used herein refers to either a polypeptide or a dimer (i.e, two) or multimer (i.e., three or more) of single chain polypeptides. The single chain polypeptides of a protein may be joined by a covalent bond, e.g., a disulfide bond, or non-covalent interactions.

The terms “polynucleotide” and “nucleic acid” interchangeably refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. Examples of polynucleotides contemplated herein include single- and double-stranded DNA, single- and double-stranded RNA, and hybrid molecules having mixtures of single- and double-stranded DNA and RNA.

The terms “conservative substitution” and “conservative mutation” refer to an alteration that results in the substitution of an amino acid with another amino acid that can be categorized as having a similar feature. Examples of categories of conservative amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R), and His (Histidine or H); an “aromatic group” including Phe (Phenylalanine or F), Tyr (Tyrosine or Y), Trp (Tryptophan or W), and (Histidine or H); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T), and Cys (Cysteine or C). Within each group, subgroups can also be identified. For example, the group of charged or polar amino acids can be sub-divided into sub-groups including: a “positively-charged sub-group” comprising Lys, Arg and His; a “negatively-charged sub-group” comprising Glu and Asp; and a “polar sub-group” comprising Asn and Gln. In another example, the aromatic or cyclic group can be sub-divided into sub-groups including: a “nitrogen ring sub-group” comprising Pro, His and Trp; and a “phenyl sub-group” comprising Phe and Tyr. In another further example, the aliphatic group can be sub-divided into sub-groups, e.g., an “aliphatic non-polar sub-group” comprising Val, Leu, Gly, and Ala; and an “aliphatic slightly-polar sub-group” comprising Met, Ser, Thr, and Cys. Examples of categories of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free-OH can be maintained; and Gln for Asn or vice versa, such that a free —NH₂ can be maintained. In some embodiments, hydrophobic amino acids are substituted for naturally occurring hydrophobic amino acid, e.g., in the active site, to preserve hydrophobicity.

The terms “identical” or percent “identity,” in the context of two or more polypeptide sequences, refer to two or more sequences or subsequences that are the same or have a specified percentage of amino acid residues, e.g., at least 60%, at least 65%, at least 70%, at least 75%, at least 80%, at least 85%, at least 90%, or at least 95% or greater, that are identical over a specified region when compared and aligned for maximum correspondence over a comparison window or designated region as measured using a sequence comparison algorithm or by manual alignment and visual inspection.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif.) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a modified Fc polypeptide “corresponds to” an amino acid in SEQ ID NO:95, when the residue aligns with the amino acid in SEQ ID NO:95 when optimally aligned to SEQ ID NO:95. The polypeptide that is aligned to the reference sequence need not be the same length as the reference sequence.

The terms “subject,” “individual,” and “patient,” as used interchangeably herein, refer to a mammal, including but not limited to humans, non-human primates, rodents (e.g., rats, mice, and guinea pigs), rabbits, cows, pigs, horses, and other mammalian species. In one embodiment, the patient is a human.

The terms “treatment,” “treating,” and the like are used herein to generally mean obtaining a desired pharmacologic and/or physiologic effect. “Treating” or “treatment” may refer to any indicia of success in the treatment or amelioration of a cancer (e.g., a HER2-positive and/or metastatic cancer), including any objective or subjective parameter such as abatement, remission, improvement in patient survival, increase in survival time or rate, diminishing of symptoms or making the disease more tolerable to the patient, slowing in the rate of degeneration or decline, or improving a patient's physical or mental well-being. The treatment or amelioration of symptoms can be based on objective or subjective parameters. The effect of treatment can be compared to an individual or pool of individuals not receiving the treatment, or to the same patient prior to treatment or at a different time during treatment.

The term “pharmaceutically acceptable excipient” refers to a non-active pharmaceutical ingredient that is biologically or pharmacologically compatible for use in humans or animals, such as, but not limited to a buffer, carrier, or preservative.

As used herein, a “therapeutic amount” or “therapeutically effective amount” of a molecule (e.g., an antibody as described herein) is an amount of the molecule that treats, alleviates, abates, or reduces the severity of symptoms of a disease in a subject.

The term “administer” refers to a method of delivering molecules or compositions to the desired site of biological action. These methods include, but are not limited to, topical delivery, parenteral delivery, intravenous delivery, intradermal delivery, intramuscular delivery, intrathecal delivery, colonic delivery, rectal delivery, or intraperitoneal delivery. In one embodiment, an antibody as described herein is administered intravenously.

### III. Anti-HER2 Antibodies

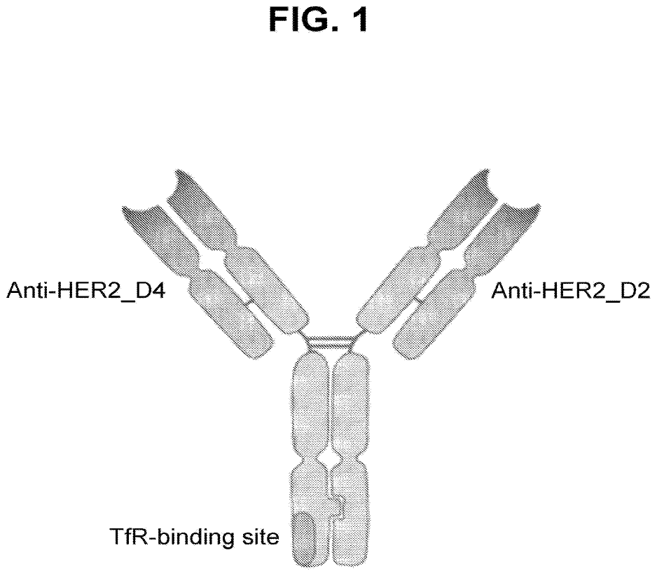

In one aspect, antibodies that bind to both subdomain II and subdomain IV of human HER2 comprising a common light chain polypeptide are provided. In some embodiments, one or both of the Fc polypeptides of the antibodies is a modified Fc polypeptide (e.g., modified to promote TfR binding and/or to enhance heterodimerization of the Fc polypeptides). A schematic drawing of such a bispecific antibody is shown in FIG. 1.

In some embodiments, an anti-HER2 antibody comprises:
(a) a first antigen binding site for human HER2 subdomain IV;
(b) a second antigen binding site for human HER2 subdomain II; and
(c) a modified Fc polypeptide dimer comprising a first Fc polypeptide that contains modifications that create a TfR-binding site,
wherein a light chain polypeptide sequence in the first antigen binding site is identical to a light chain polypeptide sequence in the second antigen binding site.

In other embodiments, an anti-HER2 antibody comprises:
(a) a first antigen binding site for human HER2 subdomain II;
(b) a second antigen binding site for human HER2 subdomain IV; and
(c) a modified Fc polypeptide dimer comprising a first Fc polypeptide that contains modifications that create a TfR-binding site,
wherein a light chain polypeptide sequence in the first antigen binding site is identical to a light chain polypeptide sequence in the second antigen binding site.

In some embodiments, the first Fc polypeptide comprises a modified CH3 domain comprising the TfR-binding site. In certain embodiments, the modified CH3 domain comprises substitutions in a set of amino acid positions as described herein that create the TfR-binding site.

In some embodiments, the first Fc polypeptide and the second Fc polypeptide each comprises modifications that promote heterodimerization. For example, the first Fc polypeptide can comprise a T366W substitution and the second Fc polypeptide can comprise T366S, L368A, and Y407V substitutions, according to EU numbering. In another example, the first Fc polypeptide can comprise T366S, L368A, and Y407V substitutions and the second Fc polypeptide can comprise a T366W substitution, according to EU numbering. Further, the first Fc polypeptide and/or the second Fc polypeptide independently can comprise modifications that reduce TfR-mediated effector function, i.e., reduce effector function upon TfR binding. For example, the modifications that reduce TfR-mediated effector function are (i) L234A and L235A substitutions or (ii) L234A and L235A substitutions and a P329G or a P329S substitution, according to EU numbering.

In some embodiments, the first Fc polypeptide and/or the second Fc polypeptide independently comprises a S239D and/or a I332E substitution, according to EU numbering. In certain embodiments, the first Fc polypeptide or the second Fc polypeptide comprises a S239D and/or a I332E substitution, according to EU numbering. In certain other embodiments, the first Fc polypeptide comprises a S239D and/or a I332E substitution and the second Fc polypeptide comprises a S239D and/or a I332E substitution, according to EU numbering. In particular embodiments, the first Fc polypeptide and/or the second Fc polypeptide independently comprising the S239D and/or the I332E substitution is capable of enhancing HER2-mediated effector function, i.e., enhancing effector function upon HER2 binding.

In some embodiments, the first Fc polypeptide comprises a S239D substitution and the second Fc polypeptide comprises a S239D substitution, according to EU numbering. In some embodiments, the first Fc polypeptide comprises a I332E substitution and the second Fc polypeptide comprises a S239D substitution, according to EU numbering. In some embodiments, the first Fc polypeptide comprises a S239D and a I332E substitution and the second Fc polypeptide comprises a S239D substitution, according to EU numbering. In some embodiments, the second Fc polypeptide comprises a S239D substitution, according to EU numbering. In some embodiments, the first Fc polypeptide comprises a S239D substitution and the second Fc polypeptide comprises a I332E substitution, according to EU numbering. In some embodiments, the first Fc polypeptide comprises a I332E substitution and the second Fc polypeptide comprises a I332E substitution, according to EU numbering. In some embodiments, the first Fc polypeptide comprises a S239D and a I332E substitution and the second Fc polypeptide comprises a I332E substitution, according to EU numbering. In some embodiments, the second Fc polypeptide comprises a I332E substitution, according to EU numbering. In some embodiments, the first Fc polypeptide comprises a S239D substitution and the second Fc polypeptide comprises a S239D and a I332E substitution, according to EU numbering. In some embodiments, the first Fc polypeptide comprises a I332E substitution and the second Fc polypeptide comprises a S239D and a I332E substitution, according to EU numbering. In some embodiments, the first Fc polypeptide comprises a S239D and a I332E substitution and the second Fc polypeptide comprises a S239D and a I332E substitution, according to EU numbering. In some embodiments, the second Fc polypeptide comprises a S239D and a I332E substitution, according to EU numbering. In some embodiments, the first Fc polypeptide comprises a S239D substitution, according to EU numbering. In some embodiments, the first Fc polypeptide comprises a I332E substitution, according to EU numbering. In some embodiments, the first Fc polypeptide comprises a S239D and a I332E substitution, according to EU numbering.

In certain embodiments, the first Fc polypeptide comprises a I332E substitution and the second Fc polypeptide comprises a S239D substitution, according to EU numbering. In particular embodiments, the first Fc polypeptide comprises a I332E substitution and a serine at position 239, and the second Fc polypeptide comprises a S239D substitution and a isoleucine at position 332, according to EU numbering.

In certain embodiments, the first Fc polypeptide comprises a S239D and a I332E substitution and the second Fc polypeptide comprises a S239D substitution, according to EU numbering. In particular embodiments, the first Fc polypeptide comprises a S239D and a I332E substitution, and the second Fc polypeptide comprises a S239D substitution and a isoleucine at position 332, according to EU numbering.

In certain embodiments, the first Fc polypeptide comprises a S239D substitution and the second Fc polypeptide comprises a I332E substitution, according to EU numbering. In particular embodiments, the first Fc polypeptide comprises a S239D substitution and a isoleucine at position 332, and the second Fc polypeptide comprises a I332E substitution and a serine at position 239, according to EU numbering.

In certain embodiments, the second Fc polypeptide comprises a I332E substitution, according to EU numbering. In particular embodiments, the first Fc polypeptide comprises a serine at position 239 and a isoleucine at 332, and the second Fc polypeptide comprises a I332E substitution and a serine at position 239, according to EU numbering.

In certain embodiments, the first Fc polypeptide comprises a S239D substitution and the second Fc polypeptide comprises a S239D and a I332E substitution, according to EU numbering. In particular embodiments, the first Fc polypeptide comprises a S239D substitution and a isoleucine at position 332, and the second Fc polypeptide comprises a S239D and a I332E substitution, according to EU numbering.

In certain embodiments, the first Fc polypeptide comprises a I332E substitution, according to EU numbering. In particular embodiments, the first Fc polypeptide comprises a I332E substitution and a serine at position 239, according to EU numbering, and the second Fc polypeptide comprises a serine at position 239 and a isoleucine at 332.

In some embodiments, the first Fc polypeptide comprises the TfR-binding site, a T366W substitution, L234A and L235A substitutions (optionally including a P329G or a P329S substitution), and optionally a S239D and/or a I332E substitution, according to EU numbering, and the second Fc polypeptide comprises T366S, L368A, and Y407V substitutions and optionally a S239D and/or a I332E substitution, according to EU numbering. For example, the first Fc polypeptide can comprise a sequence having at least 90% (e.g., 91%, 92%, 93%, 94%, 95%, 96%, 97%, 98%, 99%, or 100%) identity to the sequence of any one of SEQ ID NOS:74-84, 86 and 98, and the second Fc polypeptide can comprise a sequence having at least 90% (e.g., 91%, 92%, 93%, 94%, 95%, 96%, 97%, 98%, 99%, or 100%) identity to the sequence of any one of SEQ ID NOS:71-73, 85, and 99-100.

In certain embodiments, the first Fc polypeptide comprises the TfR-binding site and contains L234A and L235A substitutions (optionally including a P329G or a P329S substitution) and the second Fc polypeptide does not include the L234A or L325A substitutions (or the P329G or P329S substitution if present in the first Fc polypeptide), according to EU numbering. In certain other embodiments, the first Fc polypeptide comprises the TfR-binding site and does not include the L234A or L325A substitutions (or the P329G or P329S substitution if present in the second Fc polypeptide) and the second Fc polypeptide contains L234A and L235A substitutions (optionally including a P329G or a P329S substitution), according to EU numbering.

In some embodiments, one or both of the Fc polypeptides can have its C-terminal lysine removed (e.g., the Lys residue at position 447 of the Fc polypeptide, according to EU numbering). In some embodiments, removal of the C-terminal lysines in the Fc polypeptides can improve the stability of the antibody.

In some embodiments, the antigen binding site for human HER2 subdomain II in the anti-HER2 antibody comprises one or more (e.g., one, two, or all three) CDRs selected from the group consisting of:
(a) a heavy chain CDR1 comprising the amino acid sequence of SEQ ID NO:89;
(b) a heavy chain CDR2 comprising the amino acid sequence of SEQ ID NO:90; and
(c) a heavy chain CDR3 comprising the amino acid sequence of SEQ ID NO:91.

In certain embodiments, at least one of: X₁ in SEQ ID NO:89 is not T; X₂ in SEQ ID NO:89 is not F; X₃ in SEQ ID NO:89 is not T; X₁ in SEQ ID NO:90 is not N; X₂ in SEQ ID NO:90 is not N; X₃ in SEQ ID NO:90 is not S; X₄ in SEQ ID NO:90 is not G; X₅ in SEQ ID NO:90 is not G; X₆ in SEQ ID NO:90 is not Q; X₁ in SEQ ID NO:91 is not L; X₂ in SEQ ID NO:91 is not G; X₃ in SEQ ID NO:91 is not P; and X₄ in SEQ ID NO:91 is not S.

In some embodiments, the heavy chain CDR1 comprises the amino acid sequence of SEQ ID NO:89, wherein X₁ is N, K, M, or H. In some embodiments, the heavy chain CDR2 comprises the amino acid sequence of SEQ ID NO:90, wherein X₅ is Q. In some embodiments, the heavy chain CDR2 comprises the amino acid sequence of SEQ ID NO:90, wherein X₆ is R, H, or T. In some embodiments, the heavy chain CDR3 comprises the amino acid sequence of SEQ ID NO:91, wherein X₄ is W, F, D, L, or Y. In some embodiments, the heavy chain CDR3 comprises the amino acid sequence of SEQ ID NO:91, wherein X₄ is L.

In some embodiments, the antigen binding site for human HER2 subdomain II in the anti-HER2 antibody comprises one or more (e.g., one, two, or all three) CDRs selected from the group consisting of:
(a) a heavy chain CDR1 comprising the amino acid sequence of SEQ ID NO:89;
(b) a heavy chain CDR2 comprising the amino acid sequence of SEQ ID NO:90, wherein X₅ is Q; and
(c) a heavy chain CDR3 comprising the amino acid sequence of SEQ ID NO:91, wherein X₄ is L.

In some embodiments, the antigen binding site for human HER2 subdomain II in the anti-HER2 antibody comprises one or more (e.g., one, two, or all three) CDRs selected from the group consisting of:
(a) a heavy chain CDR1 having at least 90% (e.g., 91%, 92%, 93%, 94%, 95%, 96%, 97%, 98%, 99%, or 100%) sequence identity to an amino acid sequence selected from the group consisting of SEQ ID NOS:4 and 49-52 or having up to two amino acid substitutions relative to an amino acid sequence selected from the group consisting of SEQ ID NOS:4 and 49-52;
(b) a heavy chain CDR2 having at least 90% (e.g., 91%, 92%, 93%, 94%, 95%, 96%, 97%, 98%, 99%, or 100%) sequence identity to an amino acid sequence selected from the group consisting of SEQ ID NOS:5-6 and 53-55 or having up to two amino acid substitutions relative to an amino acid sequence selected from the group consisting of SEQ ID NOS:5-6 and 53-55; and
(c) a heavy chain CDR3 having at least 90% (e.g., 91%, 92%, 93%, 94%, 95%, 96%, 97%, 98%, 99%, or 100%) sequence identity to an amino acid sequence selected from the group consisting of SEQ ID NOS:7-8 and 56-59 or having up to two amino acid substitutions relative to an amino acid sequence selected from the group consisting of SEQ ID NOS:7-8 and 56-59.

In some embodiments, the antigen binding site for human HER2 subdomain II in the anti-HER2 antibody comprises one or more (e.g., one, two, or all three) CDRs selected from the group consisting of:
(a) a heavy chain CDR1 having at least 90% (e.g., 91%, 92%, 93%, 94%, 95%, 96%, 97%, 98%, 99%, or 100%) sequence identity to the amino acid sequence of SEQ ID NO:4 or having up to two amino acid substitutions relative to the amino acid sequence of SEQ ID NO:4;
(b) a heavy chain CDR2 having at least 90% (e.g., 91%, 92%, 93%, 94%, 95%, 96%, 97%, 98%, 99%, or 100%) sequence identity to the amino acid sequence of SEQ ID NO:5 or SEQ ID NO:6 or having up to two amino acid substitutions relative to the amino acid sequence of SEQ ID NO:5 or SEQ ID NO:6; and
(c) a heavy chain CDR3 having at least 90% (e.g., 91%, 92%, 93%, 94%, 95%, 96%, 97%, 98%, 99%, or 100%) sequence identity to the amino acid sequence of SEQ ID NO:7 or SEQ ID NO:8 or having up to two amino acid substitutions relative to the amino acid sequence of SEQ ID NO:7 or SEQ ID NO:8.

In some embodiments, the antigen binding site for human HER2 subdomain II in the anti-HER2 antibody comprises one or more (e.g., one, two, or all three) CDRs selected from the group consisting of:
(a) a heavy chain CDR1 comprising the amino acid sequence of SEQ ID NO:4;
(b) a heavy chain CDR2 comprising the amino acid sequence of SEQ ID NO:5 or SEQ ID NO:6; and
(c) a heavy chain CDR3 comprising the amino acid sequence of SEQ ID NO:7 or SEQ ID NO:8.

In some embodiments, the antigen binding site for human HER2 subdomain II in the anti-HER2 antibody comprises one or more (e.g., one, two, or all three) CDRs selected from the group consisting of:
(a) a heavy chain CDR1 comprising the amino acid sequence of SEQ ID NO:4;
(b) a heavy chain CDR2 comprising the amino acid sequence of SEQ ID NO:6; and
(c) a heavy chain CDR3 comprising the amino acid sequence of SEQ ID NO:7.

In some embodiments, the antigen binding site for human HER2 subdomain II in the anti-HER2 antibody comprises one or more (e.g., one, two, or all three) CDRs selected from the group consisting of:
(a) a heavy chain CDR1 comprising the amino acid sequence of SEQ ID NO:4;
(b) a heavy chain CDR2 comprising the amino acid sequence of SEQ ID NO:5; and
(c) a heavy chain CDR3 comprising the amino acid sequence of SEQ ID NO:8.

In some embodiments, the antigen binding site for human HER2 subdomain II in the anti-HER2 antibody comprises one or more (e.g., one, two, or all three) CDRs selected from the group consisting of:
(a) a heavy chain CDR1 comprising the amino acid sequence of SEQ ID NO:4;
(b) a heavy chain CDR2 comprising the amino acid sequence of SEQ ID NO:6; and
(c) a heavy chain CDR3 comprising the amino acid sequence of SEQ ID NO:8.

In some embodiments, the antigen binding site for human HER2 subdomain II in the anti-HER2 antibody comprises a heavy chain variable region comprising an amino acid sequence having at least 90% (e.g., 91%, 92%, 93%, 94%, 95%, 96%, 97%, 98%, 99%, or 100%) sequence identity to any one of SEQ ID NOS:1-3 and 60-70. In some embodiments, the antigen binding site for human HER2 subdomain II in the anti-HER2 antibody comprises a heavy chain variable region comprising the amino acid sequence of any one of SEQ ID NOS:1-3 and 60-70.

In some embodiments, the antigen binding site for human HER2 subdomain IV in the anti-HER2 antibody comprises one or more (e.g., one, two, or all three) CDRs selected from the group consisting of:
(a) a heavy chain CDR1 comprising the amino acid sequence of SEQ ID NO:16 or having up to two amino acid substitutions relative to the amino acid sequence of SEQ ID NO:16;
(b) a heavy chain CDR2 having at least 90% (e.g., 91%, 92%, 93%, 94%, 95%, 96%, 97%, 98%, 99%, or 100%) sequence identity to the amino acid sequence of SEQ ID NO:17 or having up to two amino acid substitutions relative to the amino acid sequence of SEQ ID NO:17; and
(c) a heavy chain CDR3 having at least 90% (e.g., 91%, 92%, 93%, 94%, 95%, 96%, 97%, 98%, 99%, or 100%) sequence identity to the amino acid sequence of SEQ ID NO:18 or having up to two amino acid substitutions relative to the amino acid sequence of SEQ ID NO:18.

In some embodiments, the antigen binding site for human HER2 subdomain IV in the anti-HER2 antibody comprises one or more (e.g., one, two, or all three) CDRs selected from the group consisting of:
(a) a heavy chain CDR1 comprising the amino acid sequence of SEQ ID NO:16;
(b) a heavy chain CDR2 comprising the amino acid sequence of SEQ ID NO:17; and
(c) a heavy chain CDR3 comprising the amino acid sequence of SEQ ID NO:18.

In some embodiments, the antigen binding site for human HER2 subdomain IV in the anti-HER2 antibody comprises a heavy chain variable region comprising an amino acid sequence having at least 90% (e.g., 91%, 92%, 93%, 94%, 95%, 96%, 97%, 98%, 99%, or 100%) sequence identity to SEQ ID NO:15. In some embodiments, the antigen binding site for human HER2 subdomain IV in the anti-HER2 antibody comprises a heavy chain variable region comprising the sequence of SEQ ID NO:15.

In some embodiments, the light chain polypeptide sequence in the first and second antigen binding sites, i.e., one for HER2 subdomain II and the other for HER2 subdomain IV, in the anti-HER2 antibody comprises one or more (e.g., one, two, or all three) CDRs selected from the group consisting of:
(a) a light chain CDR1 having at least 90% (e.g., 91%, 92%, 93%, 94%, 95%, 96%, 97%, 98%, 99%, or 100%) sequence identity to the amino acid sequence of SEQ ID NO:11 or having up to two amino acid substitutions relative to the amino acid sequence of SEQ ID NO:11;
(b) a light chain CDR2 having up to two amino acid substitutions relative to the amino acid sequence of SEQ ID NO:12; and
(c) a light chain CDR3 having up to two amino acid substitutions relative to the amino acid sequence of SEQ ID NO:13 or 14.

In some embodiments, the light chain polypeptide sequence comprises one or more (e.g., one, two, or all three) CDRs selected from the group consisting of:
(a) a light chain CDR1 comprising the amino acid sequence of SEQ ID NO:11;
(b) a light chain CDR2 comprising the amino acid sequence of SEQ ID NO:12; and
(c) a light chain CDR3 comprising the amino acid sequence of SEQ ID NO:13 or 14.

In some embodiments, the light chain polypeptide sequence comprises one or more (e.g., one, two, or all three) CDRs selected from the group consisting of:
(a) a light chain CDR1 comprising the amino acid sequence of SEQ ID NO:11;
(b) a light chain CDR2 comprising the amino acid sequence of SEQ ID NO:12; and
(c) a light chain CDR3 comprising the amino acid sequence of SEQ ID NO:13.

In some embodiments, the light chain polypeptide sequence comprises one or more (e.g., one, two, or all three) CDRs selected from the group consisting of:
(a) a light chain CDR1 comprising the amino acid sequence of SEQ ID NO:11;
(b) a light chain CDR2 comprising the amino acid sequence of SEQ ID NO:12; and
(c) a light chain CDR3 comprising the amino acid sequence of SEQ ID NO:14.

In certain embodiments, the light chain polypeptide sequence comprises a light chain CDR3 comprising the amino acid sequence of SEQ ID NO:13 or 14 and optionally further comprises one or more (e.g., one or both) CDRs selected from the group consisting of: a light chain CDR1 having at least 90% (e.g., 91%, 92%, 93%, 94%, 95%, 96%, 97%, 98%, 99%, or 100%) sequence identity to the amino acid sequence of SEQ ID NO:11 or having up to two amino acid substitutions relative to the amino acid sequence of SEQ ID NO:11; and a light chain CDR2 having up to two amino acid substitutions relative to the amino acid sequence of SEQ ID NO:12. In particular embodiments, the light chain polypeptide sequence comprises the amino acid sequence of SEQ ID NO:13 or 14 and optionally further comprises one or more (e.g., one or both) CDRs selected from the group consisting of: a light chain CDR1 comprising the amino acid sequence of SEQ ID NO:11; and a light chain CDR2 comprising the amino acid sequence of SEQ ID NO:12.

In some embodiments, the light chain polypeptide sequence comprises a light chain variable region comprising an amino acid sequence having at least 90% (e.g., 91%, 92%, 93%, 94%, 95%, 96%, 97%, 98%, 99%, or 100%) sequence identity to any one of SEQ ID NOS:9-10. In some embodiments, the light chain polypeptide sequence comprises a light chain variable region comprising the amino acid sequence of any one of SEQ ID NOS:9-10.

In some embodiments, the anti-HER2 antibody comprises heavy and light chain CDRs selected from the combinations listed in Table 1, i.e., any one of Combo #A-AC. In particular embodiments of this antibody:
(a) the first heavy chain comprises a heavy chain CDR1 comprising the amino acid sequence of SEQ ID NO:16, a heavy chain CDR2 comprising the amino acid sequence of SEQ ID NO:17, and a heavy chain CDR3 comprising the amino acid sequence of SEQ ID NO:18;
(b) the second heavy chain comprises a heavy chain CDR1 comprising the amino acid sequence of any one of SEQ ID NOS:4 and 49-52, a heavy chain CDR2 comprising the amino acid sequence of any one of SEQ ID NOS:5-6 and 53-55, and a heavy chain CDR3 comprising the amino acid sequence of any one of SEQ ID NOS:7-8 and 56-59; and
(c) the light chain comprises a light chain CDR1 comprising the amino acid sequence of SEQ ID NO:11, a light chain CDR2 comprising the amino acid sequence of SEQ ID NO:12, and a light chain CDR3 comprising the amino acid sequence of any one of SEQ ID NOS:13-14.

In certain embodiments of the anti-HER2 antibody, the first antigen binding site comprises an amino acid sequence selected from the group consisting of SEQ ID NO:15; the second antigen binding site comprises the amino acid sequence of SEQ ID NOS:1-3 and 60-70; the first Fc polypeptide comprises an amino acid sequence selected from the group consisting of SEQ ID NOS:74-84, 86, and 98; and the light chain polypeptide sequence comprises an amino acid sequence selected from the group consisting of SEQ ID NOS:9-10 and 19. In some embodiments, the antibody further comprises a second Fc polypeptide comprising an amino acid sequence selected from the group consisting of SEQ ID NOS:71-73, 85, and 99-100.

In certain other embodiments of the anti-HER2 antibody, the first antigen binding site comprises an amino acid sequence selected from the group consisting of SEQ ID NOS:1-3 and 60-70; the second antigen binding site comprises the amino acid sequence of SEQ ID NO:15; the first Fc polypeptide comprises an amino acid sequence selected from the group consisting of SEQ ID NOS:74-84, 86, and 98; and the light chain polypeptide sequence comprises an amino acid sequence selected from the group consisting of SEQ ID NOS:9-10 and 19. In some embodiments, the antibody further comprises a second Fc polypeptide comprising an amino acid sequence selected from the group consisting of SEQ ID NOS:71-73, 85, and 99-100.

In some embodiments, the anti-HER2 antibody comprises a first heavy chain for binding to human HER2 subdomain II or IV, a second heavy chain for binding to the other HER2 subdomain, and two identical light chains.

In certain embodiments, the anti-HER2 antibody comprises heavy and light chains each having at least 90% (e.g., 91%, 92%, 93%, 94%, 95%, 96%, 97%, 98%, 99%, or 100%) sequence identity to the amino acid sequences selected from the combinations listed in Table 2, i.e., any one of Combo #A-AT.

In particular embodiments of this antibody:
(a) the first heavy chain comprises SEQ ID NO:37, the second heavy chain comprises SEQ ID NO:25, and the light chain comprises SEQ ID NO:9;
(b) the first heavy chain comprises SEQ ID NO:31, the second heavy chain comprises SEQ ID NO:30, and the light chain comprises SEQ ID NO:9;
(c) the first heavy chain comprises SEQ ID NO:31, the second heavy chain comprises SEQ ID NO:29, and the light chain comprises SEQ ID NO:9;
(d) the first heavy chain comprises SEQ ID NO:38, the second heavy chain comprises SEQ ID NO:30, and the light chain comprises SEQ ID NO:9;
(e) the first heavy chain comprises SEQ ID NO:32, the second heavy chain comprises SEQ ID NO:30, and the light chain comprises SEQ ID NO:9;
(f) the first heavy chain comprises SEQ ID NO:32, the second heavy chain comprises SEQ ID NO:29, and the light chain comprises SEQ ID NO:9;
(g) the first heavy chain comprises SEQ ID NO:39, the second heavy chain comprises SEQ ID NO:30, and the light chain comprises SEQ ID NO:9;
(h) the first heavy chain comprises SEQ ID NO:37, the second heavy chain comprises SEQ ID NO:26, and the light chain comprises SEQ ID NO:9;
(i) the first heavy chain comprises SEQ ID NO:31, the second heavy chain comprises SEQ ID NO:34, and the light chain comprises SEQ ID NO:9;
(j) the first heavy chain comprises SEQ ID NO:31, the second heavy chain comprises SEQ ID NO:33, and the light chain comprises SEQ ID NO:9;
(k) the first heavy chain comprises SEQ ID NO:38, the second heavy chain comprises SEQ ID NO:34, and the light chain comprises SEQ ID NO:9;
(l) the first heavy chain comprises SEQ ID NO:32, the second heavy chain comprises SEQ ID NO:34, and the light chain comprises SEQ ID NO:9;
(m) the first heavy chain comprises SEQ ID NO:32, the second heavy chain comprises SEQ ID NO:33, and the light chain comprises SEQ ID NO:9;
(n) the first heavy chain comprises SEQ ID NO:39, the second heavy chain comprises SEQ ID NO:34, and the light chain comprises SEQ ID NO:9;
(o) the first heavy chain comprises SEQ ID NO:37, the second heavy chain comprises SEQ ID NO:27, and the light chain comprises SEQ ID NO:9;
(p) the first heavy chain comprises SEQ ID NO:31, the second heavy chain comprises SEQ ID NO:36, and the light chain comprises SEQ ID NO:9;
(q) the first heavy chain comprises SEQ ID NO:31, the second heavy chain comprises SEQ ID NO:35, and the light chain comprises SEQ ID NO:9;
(r) the first heavy chain comprises SEQ ID NO:38, the second heavy chain comprises SEQ ID NO:36, and the light chain comprises SEQ ID NO:9;
(s) the first heavy chain comprises SEQ ID NO:32, the second heavy chain comprises SEQ ID NO:36, and the light chain comprises SEQ ID NO:9;
(t) the first heavy chain comprises SEQ ID NO:32, the second heavy chain comprises SEQ ID NO:35, and the light chain comprises SEQ ID NO:9;
(u) the first heavy chain comprises SEQ ID NO:39, the second heavy chain comprises SEQ ID NO:46, and the light chain comprises SEQ ID NO:9;
(v) the first heavy chain comprises SEQ ID NO:37, the second heavy chain comprises SEQ ID NO:25, and the light chain comprises SEQ ID NO:10;
(w) the first heavy chain comprises SEQ ID NO:31, the second heavy chain comprises SEQ ID NO:30, and the light chain comprises SEQ ID NO:10;
(x) the first heavy chain comprises SEQ ID NO:31, the second heavy chain comprises SEQ ID NO:29, and the light chain comprises SEQ ID NO:10;
(y) the first heavy chain comprises SEQ ID NO:38, the second heavy chain comprises SEQ ID NO:30, and the light chain comprises SEQ ID NO:10;
(z) the first heavy chain comprises SEQ ID NO:32, the second heavy chain comprises SEQ ID NO:30, and the light chain comprises SEQ ID NO:10;
(aa) the first heavy chain comprises SEQ ID NO:32, the second heavy chain comprises SEQ ID NO:29, and the light chain comprises SEQ ID NO:10;
(ab) the first heavy chain comprises SEQ ID NO:39, the second heavy chain comprises SEQ ID NO:30, and the light chain comprises SEQ ID NO:10;
(ac) the first heavy chain comprises SEQ ID NO:37, the second heavy chain comprises SEQ ID NO:26, and the light chain comprises SEQ ID NO:10;
(ad) the first heavy chain comprises SEQ ID NO:31, the second heavy chain comprises SEQ ID NO:34, and the light chain comprises SEQ ID NO:10;
(ae) the first heavy chain comprises SEQ ID NO:31, the second heavy chain comprises SEQ ID NO:33, and the light chain comprises SEQ ID NO:10;
(af) the first heavy chain comprises SEQ ID NO:38, the second heavy chain comprises SEQ ID NO:34, and the light chain comprises SEQ ID NO:10;
(ag) the first heavy chain comprises SEQ ID NO:32, the second heavy chain comprises SEQ ID NO:34, and the light chain comprises SEQ ID NO:10;
(ah) the first heavy chain comprises SEQ ID NO:32, the second heavy chain comprises SEQ ID NO:33, and the light chain comprises SEQ ID NO:10;
(ai) the first heavy chain comprises SEQ ID NO:39, the second heavy chain comprises SEQ ID NO:34, and the light chain comprises SEQ ID NO:10;
(aj) the first heavy chain comprises SEQ ID NO:37, the second heavy chain comprises SEQ ID NO:27, and the light chain comprises SEQ ID NO:10;
(ak) the first heavy chain comprises SEQ ID NO:31, the second heavy chain comprises SEQ ID NO:36, and the light chain comprises SEQ ID NO:10;
(al) the first heavy chain comprises SEQ ID NO:31, the second heavy chain comprises SEQ ID NO:35, and the light chain comprises SEQ ID NO:10;
(am) the first heavy chain comprises SEQ ID NO:38, the second heavy chain comprises SEQ ID NO:36, and the light chain comprises SEQ ID NO:10;
(an) the first heavy chain comprises SEQ ID NO:32, the second heavy chain comprises SEQ ID NO:36, and the light chain comprises SEQ ID NO:10;
(ao) the first heavy chain comprises SEQ ID NO:32, the second heavy chain comprises SEQ ID NO:35, and the light chain comprises SEQ ID NO:10;
(ap) the first heavy chain comprises SEQ ID NO:39, the second heavy chain comprises SEQ ID NO:46, and the light chain comprises SEQ ID NO:10;
(aq) the first heavy chain comprises SEQ ID NO:20, the second heavy chain comprises SEQ ID NO:24, and the light chain comprises SEQ ID NO:19;
(ar) the first heavy chain comprises SEQ ID NO:21, the second heavy chain comprises SEQ ID NO:24, and the light chain comprises SEQ ID NO:19;
(as) the first heavy chain comprises SEQ ID NO:22, the second heavy chain comprises SEQ ID NO:24, and the light chain comprises SEQ ID NO:19; or
(at) the first heavy chain comprises SEQ ID NO:23, the second heavy chain comprises SEQ ID NO:24, and the light chain comprises SEQ ID NO:19.

In certain embodiments, the anti-HER2 antibody comprises a first heavy chain comprising a V_{H} and a Fc sequence each having at least 90% (e.g., 91%, 92%, 93%, 94%, 95%, 96%, 97%, 98%, 99%, or 100%) sequence identity to the amino acid sequences selected from the combinations in Table 3, i.e., any one of Combo #A-L, and a second heavy chain comprising a V_{H} and a Fc sequence each having at least 90% (e.g., 91%, 92%, 93%, 94%, 95%, 96%, 97%, 98%, 99%, or 100%) sequence identity to the amino acid sequences selected from the combinations in Table 4, i.e., any one of Combo #A-L. In any of these heavy chain combinations, the light chain polypeptide sequence has at least 90% (e.g., 91%, 92%, 93%, 94%, 95%, 96%, 97%, 98%, 99%, or 100%) sequence identity to an amino acid sequence selected from the group consisting of SEQ ID NOS:9-10 and 19.

In particular embodiments of this antibody:
(a) the first heavy chain comprises SEQ ID NOS:1 and 71 and the second heavy chain comprises SEQ ID NOS:15 and 86;
(b) the first heavy chain comprises SEQ ID NOS:1 and 71 and the second heavy chain comprises SEQ ID NOS:15 and 74;
(c) the first heavy chain comprises SEQ ID NOS:1 and 71 and the second heavy chain comprises SEQ ID NOS:15 and 75;
(d) the first heavy chain comprises SEQ ID NOS:1 and 71 and the second heavy chain comprises SEQ ID NOS:15 and 76;
(e) the first heavy chain comprises SEQ ID NOS:1 and 71 and the second heavy chain comprises SEQ ID NOS:15 and 77;
(f) the first heavy chain comprises SEQ ID NOS:1 and 71 and the second heavy chain comprises SEQ ID NOS:15 and 78;
(g) the first heavy chain comprises SEQ ID NOS:1 and 71 and the second heavy chain comprises SEQ ID NOS:15 and 79;
(h) the first heavy chain comprises SEQ ID NOS:1 and 71 and the second heavy chain comprises SEQ ID NOS:15 and 80;
(i) the first heavy chain comprises SEQ ID NOS:1 and 71 and the second heavy chain comprises SEQ ID NOS:15 and 81;
(j) the first heavy chain comprises SEQ ID NOS:1 and 71 and the second heavy chain comprises SEQ ID NOS:15 and 82;
(k) the first heavy chain comprises SEQ ID NOS:1 and 71 and the second heavy chain comprises SEQ ID NOS:15 and 83; or
(l) the first heavy chain comprises SEQ ID NOS:1 and 71 and the second heavy chain comprises SEQ ID NOS:15 and 84.

In particular embodiments of this antibody:
(a) the first heavy chain comprises SEQ ID NOS:2 and 71 and the second heavy chain comprises SEQ ID NOS:15 and 86;
(b) the first heavy chain comprises SEQ ID NOS:2 and 71 and the second heavy chain comprises SEQ ID NOS:15 and 74;
(c) the first heavy chain comprises SEQ ID NOS:2 and 71 and the second heavy chain comprises SEQ ID NOS:15 and 75;
(d) the first heavy chain comprises SEQ ID NOS:2 and 71 and the second heavy chain comprises SEQ ID NOS:15 and 76;
(e) the first heavy chain comprises SEQ ID NOS:2 and 71 and the second heavy chain comprises SEQ ID NOS:15 and 77;
(f) the first heavy chain comprises SEQ ID NOS:2 and 71 and the second heavy chain comprises SEQ ID NOS:15 and 78;
(g) the first heavy chain comprises SEQ ID NOS:2 and 71 and the second heavy chain comprises SEQ ID NOS:15 and 79;
(h) the first heavy chain comprises SEQ ID NOS:2 and 71 and the second heavy chain comprises SEQ ID NOS:15 and 80;
(i) the first heavy chain comprises SEQ ID NOS:2 and 71 and the second heavy chain comprises SEQ ID NOS:15 and 81;
(j) the first heavy chain comprises SEQ ID NOS:2 and 71 and the second heavy chain comprises SEQ ID NOS:15 and 82;
(k) the first heavy chain comprises SEQ ID NOS:2 and 71 and the second heavy chain comprises SEQ ID NOS:15 and 83; or
(l) the first heavy chain comprises SEQ ID NOS:2 and 71 and the second heavy chain comprises SEQ ID NOS:15 and 84.

In particular embodiments of this antibody:
(a) the first heavy chain comprises SEQ ID NOS:3 and 71 and the second heavy chain comprises SEQ ID NOS:15 and 86;
(b) the first heavy chain comprises SEQ ID NOS:3 and 71 and the second heavy chain comprises SEQ ID NOS:15 and 74;
(c) the first heavy chain comprises SEQ ID NOS:3 and 71 and the second heavy chain comprises SEQ ID NOS:15 and 75;
(d) the first heavy chain comprises SEQ ID NOS:3 and 71 and the second heavy chain comprises SEQ ID NOS:15 and 76;
(e) the first heavy chain comprises SEQ ID NOS:3 and 71 and the second heavy chain comprises SEQ ID NOS:15 and 77;
(f) the first heavy chain comprises SEQ ID NOS:3 and 71 and the second heavy chain comprises SEQ ID NOS:15 and 78;
(g) the first heavy chain comprises SEQ ID NOS:3 and 71 and the second heavy chain comprises SEQ ID NOS:15 and 79;
(h) the first heavy chain comprises SEQ ID NOS:3 and 71 and the second heavy chain comprises SEQ ID NOS:15 and 80;
(i) the first heavy chain comprises SEQ ID NOS:3 and 71 and the second heavy chain comprises SEQ ID NOS:15 and 81;
(j) the first heavy chain comprises SEQ ID NOS:3 and 71 and the second heavy chain comprises SEQ ID NOS:15 and 82;
(k) the first heavy chain comprises SEQ ID NOS:3 and 71 and the second heavy chain comprises SEQ ID NOS:15 and 83; or
(l) the first heavy chain comprises SEQ ID NOS:3 and 71 and the second heavy chain comprises SEQ ID NOS:15 and 84.

In particular embodiments of this antibody:
(a) the first heavy chain comprises SEQ ID NOS:1 and 72 and the second heavy chain comprises SEQ ID NOS:15 and 86;
(b) the first heavy chain comprises SEQ ID NOS:1 and 72 and the second heavy chain comprises SEQ ID NOS:15 and 74;
(c) the first heavy chain comprises SEQ ID NOS:1 and 72 and the second heavy chain comprises SEQ ID NOS:15 and 75;
(d) the first heavy chain comprises SEQ ID NOS:1 and 72 and the second heavy chain comprises SEQ ID NOS:15 and 76;
(e) the first heavy chain comprises SEQ ID NOS:1 and 72 and the second heavy chain comprises SEQ ID NOS:15 and 77;
(f) the first heavy chain comprises SEQ ID NOS:1 and 72 and the second heavy chain comprises SEQ ID NOS:15 and 78;
(g) the first heavy chain comprises SEQ ID NOS:1 and 72 and the second heavy chain comprises SEQ ID NOS:15 and 79;
(h) the first heavy chain comprises SEQ ID NOS:1 and 72 and the second heavy chain comprises SEQ ID NOS:15 and 80;
(i) the first heavy chain comprises SEQ ID NOS:1 and 72 and the second heavy chain comprises SEQ ID NOS:15 and 81;
(j) the first heavy chain comprises SEQ ID NOS:1 and 72 and the second heavy chain comprises SEQ ID NOS:15 and 82;
(k) the first heavy chain comprises SEQ ID NOS:1 and 72 and the second heavy chain comprises SEQ ID NOS:15 and 83; or
(l) the first heavy chain comprises SEQ ID NOS:1 and 72 and the second heavy chain comprises SEQ ID NOS:15 and 84.

In particular embodiments of this antibody:
(a) the first heavy chain comprises SEQ ID NOS:2 and 72 and the second heavy chain comprises SEQ ID NOS:15 and 86;
(b) the first heavy chain comprises SEQ ID NOS:2 and 72 and the second heavy chain comprises SEQ ID NOS:15 and 74;
(c) the first heavy chain comprises SEQ ID NOS:2 and 72 and the second heavy chain comprises SEQ ID NOS:15 and 75;
(d) the first heavy chain comprises SEQ ID NOS:2 and 72 and the second heavy chain comprises SEQ ID NOS:15 and 76;
(e) the first heavy chain comprises SEQ ID NOS:2 and 72 and the second heavy chain comprises SEQ ID NOS:15 and 77;
(f) the first heavy chain comprises SEQ ID NOS:2 and 72 and the second heavy chain comprises SEQ ID NOS:15 and 78;
(g) the first heavy chain comprises SEQ ID NOS:2 and 72 and the second heavy chain comprises SEQ ID NOS:15 and 79;
(h) the first heavy chain comprises SEQ ID NOS:2 and 72 and the second heavy chain comprises SEQ ID NOS:15 and 80;
(i) the first heavy chain comprises SEQ ID NOS:2 and 72 and the second heavy chain comprises SEQ ID NOS:15 and 81;
(j) the first heavy chain comprises SEQ ID NOS:2 and 72 and the second heavy chain comprises SEQ ID NOS:15 and 82;
(k) the first heavy chain comprises SEQ ID NOS:2 and 72 and the second heavy chain comprises SEQ ID NOS:15 and 83; or
(l) the first heavy chain comprises SEQ ID NOS:2 and 72 and the second heavy chain comprises SEQ ID NOS:15 and 84.

In particular embodiments of this antibody:
(a) the first heavy chain comprises SEQ ID NOS:3 and 72 and the second heavy chain comprises SEQ ID NOS:15 and 86;
(b) the first heavy chain comprises SEQ ID NOS:3 and 72 and the second heavy chain comprises SEQ ID NOS:15 and 74;
(c) the first heavy chain comprises SEQ ID NOS:3 and 72 and the second heavy chain comprises SEQ ID NOS:15 and 75;
(d) the first heavy chain comprises SEQ ID NOS:3 and 72 and the second heavy chain comprises SEQ ID NOS:15 and 76;
(e) the first heavy chain comprises SEQ ID NOS:3 and 72 and the second heavy chain comprises SEQ ID NOS:15 and 77;
(f) the first heavy chain comprises SEQ ID NOS:3 and 72 and the second heavy chain comprises SEQ ID NOS:15 and 78;
(g) the first heavy chain comprises SEQ ID NOS:3 and 72 and the second heavy chain comprises SEQ ID NOS:15 and 79;
(h) the first heavy chain comprises SEQ ID NOS:3 and 72 and the second heavy chain comprises SEQ ID NOS:15 and 80;
(i) the first heavy chain comprises SEQ ID NOS:3 and 72 and the second heavy chain comprises SEQ ID NOS:15 and 81;
(j) the first heavy chain comprises SEQ ID NOS:3 and 72 and the second heavy chain comprises SEQ ID NOS:15 and 82;
(k) the first heavy chain comprises SEQ ID NOS:3 and 72 and the second heavy chain comprises SEQ ID NOS:15 and 83; or
(l) the first heavy chain comprises SEQ ID NOS:3 and 72 and the second heavy chain comprises SEQ ID NOS:15 and 84.

In particular embodiments of this antibody:
(a) the first heavy chain comprises SEQ ID NOS:1 and 73 and the second heavy chain comprises SEQ ID NOS:15 and 86;
(b) the first heavy chain comprises SEQ ID NOS:1 and 73 and the second heavy chain comprises SEQ ID NOS:15 and 74;
(c) the first heavy chain comprises SEQ ID NOS:1 and 73 and the second heavy chain comprises SEQ ID NOS:15 and 75;
(d) the first heavy chain comprises SEQ ID NOS:1 and 73 and the second heavy chain comprises SEQ ID NOS:15 and 76;
(e) the first heavy chain comprises SEQ ID NOS:1 and 73 and the second heavy chain comprises SEQ ID NOS:15 and 77;
(f) the first heavy chain comprises SEQ ID NOS:1 and 73 and the second heavy chain comprises SEQ ID NOS:15 and 78;
(g) the first heavy chain comprises SEQ ID NOS:1 and 73 and the second heavy chain comprises SEQ ID NOS:15 and 79;
(h) the first heavy chain comprises SEQ ID NOS:1 and 73 and the second heavy chain comprises SEQ ID NOS:15 and 80;
(i) the first heavy chain comprises SEQ ID NOS:1 and 73 and the second heavy chain comprises SEQ ID NOS:15 and 81;
(j) the first heavy chain comprises SEQ ID NOS:1 and 73 and the second heavy chain comprises SEQ ID NOS:15 and 82;
(k) the first heavy chain comprises SEQ ID NOS:1 and 73 and the second heavy chain comprises SEQ ID NOS:15 and 83; or
(l) the first heavy chain comprises SEQ ID NOS:1 and 73 and the second heavy chain comprises SEQ ID NOS:15 and 84.

In particular embodiments of this antibody:
(a) the first heavy chain comprises SEQ ID NOS:2 and 73 and the second heavy chain comprises SEQ ID NOS:15 and 86;
(b) the first heavy chain comprises SEQ ID NOS:2 and 73 and the second heavy chain comprises SEQ ID NOS:15 and 74;
(c) the first heavy chain comprises SEQ ID NOS:2 and 73 and the second heavy chain comprises SEQ ID NOS:15 and 75;
(d) the first heavy chain comprises SEQ ID NOS:2 and 73 and the second heavy chain comprises SEQ ID NOS:15 and 76;
(e) the first heavy chain comprises SEQ ID NOS:2 and 73 and the second heavy chain comprises SEQ ID NOS:15 and 77;
(f) the first heavy chain comprises SEQ ID NOS:2 and 73 and the second heavy chain comprises SEQ ID NOS:15 and 78;
(g) the first heavy chain comprises SEQ ID NOS:2 and 73 and the second heavy chain comprises SEQ ID NOS:15 and 79;
(h) the first heavy chain comprises SEQ ID NOS:2 and 73 and the second heavy chain comprises SEQ ID NOS:15 and 80;
(i) the first heavy chain comprises SEQ ID NOS:2 and 73 and the second heavy chain comprises SEQ ID NOS:15 and 81;
(j) the first heavy chain comprises SEQ ID NOS:2 and 73 and the second heavy chain comprises SEQ ID NOS:15 and 82;
(k) the first heavy chain comprises SEQ ID NOS:2 and 73 and the second heavy chain comprises SEQ ID NOS:15 and 83; or
(l) the first heavy chain comprises SEQ ID NOS:2 and 73 and the second heavy chain comprises SEQ ID NOS:15 and 84.

In particular embodiments of this antibody:
(a) the first heavy chain comprises SEQ ID NOS:3 and 73 and the second heavy chain comprises SEQ ID NOS:15 and 86;
(b) the first heavy chain comprises SEQ ID NOS:3 and 73 and the second heavy chain comprises SEQ ID NOS:15 and 74;
(c) the first heavy chain comprises SEQ ID NOS:3 and 73 and the second heavy chain comprises SEQ ID NOS:15 and 75;
(d) the first heavy chain comprises SEQ ID NOS:3 and 73 and the second heavy chain comprises SEQ ID NOS:15 and 76;
(e) the first heavy chain comprises SEQ ID NOS:3 and 73 and the second heavy chain comprises SEQ ID NOS:15 and 77;
(f) the first heavy chain comprises SEQ ID NOS:3 and 73 and the second heavy chain comprises SEQ ID NOS:15 and 78;
(g) the first heavy chain comprises SEQ ID NOS:3 and 73 and the second heavy chain comprises SEQ ID NOS:15 and 79;
(h) the first heavy chain comprises SEQ ID NOS:3 and 73 and the second heavy chain comprises SEQ ID NOS:15 and 80;
(i) the first heavy chain comprises SEQ ID NOS:3 and 73 and the second heavy chain comprises SEQ ID NOS:15 and 81;
(j) the first heavy chain comprises SEQ ID NOS:3 and 73 and the second heavy chain comprises SEQ ID NOS:15 and 82;
(k) the first heavy chain comprises SEQ ID NOS:3 and 73 and the second heavy chain comprises SEQ ID NOS:15 and 83; or
(l) the first heavy chain comprises SEQ ID NOS:3 and 73 and the second heavy chain comprises SEQ ID NOS:15 and 84.

In particular embodiments of this antibody:
(a) the first heavy chain comprises SEQ ID NOS:1 and 85 and the second heavy chain comprises SEQ ID NOS:15 and 86;
(b) the first heavy chain comprises SEQ ID NOS:1 and 85 and the second heavy chain comprises SEQ ID NOS:15 and 74;
(c) the first heavy chain comprises SEQ ID NOS:1 and 85 and the second heavy chain comprises SEQ ID NOS:15 and 75;
(d) the first heavy chain comprises SEQ ID NOS:1 and 85 and the second heavy chain comprises SEQ ID NOS:15 and 76;
(e) the first heavy chain comprises SEQ ID NOS:1 and 85 and the second heavy chain comprises SEQ ID NOS:15 and 77;
(f) the first heavy chain comprises SEQ ID NOS:1 and 85 and the second heavy chain comprises SEQ ID NOS:15 and 78;
(g) the first heavy chain comprises SEQ ID NOS:1 and 85 and the second heavy chain comprises SEQ ID NOS:15 and 79;
(h) the first heavy chain comprises SEQ ID NOS:1 and 85 and the second heavy chain comprises SEQ ID NOS:15 and 80;
(i) the first heavy chain comprises SEQ ID NOS:1 and 85 and the second heavy chain comprises SEQ ID NOS:15 and 81;
(j) the first heavy chain comprises SEQ ID NOS:1 and 85 and the second heavy chain comprises SEQ ID NOS:15 and 82;
(k) the first heavy chain comprises SEQ ID NOS:1 and 85 and the second heavy chain comprises SEQ ID NOS:15 and 83; or
(l) the first heavy chain comprises SEQ ID NOS:1 and 85 and the second heavy chain comprises SEQ ID NOS:15 and 84.

In particular embodiments of this antibody:
(a) the first heavy chain comprises SEQ ID NOS:2 and 85 and the second heavy chain comprises SEQ ID NOS:15 and 86;
(b) the first heavy chain comprises SEQ ID NOS:2 and 85 and the second heavy chain comprises SEQ ID NOS:15 and 74;
(c) the first heavy chain comprises SEQ ID NOS:2 and 85 and the second heavy chain comprises SEQ ID NOS:15 and 75;
(d) the first heavy chain comprises SEQ ID NOS:2 and 85 and the second heavy chain comprises SEQ ID NOS:15 and 76;
(e) the first heavy chain comprises SEQ ID NOS:2 and 85 and the second heavy chain comprises SEQ ID NOS:15 and 77;
(f) the first heavy chain comprises SEQ ID NOS:2 and 85 and the second heavy chain comprises SEQ ID NOS:15 and 78;
(g) the first heavy chain comprises SEQ ID NOS:2 and 85 and the second heavy chain comprises SEQ ID NOS:15 and 79;
(h) the first heavy chain comprises SEQ ID NOS:2 and 85 and the second heavy chain comprises SEQ ID NOS:15 and 80;
(i) the first heavy chain comprises SEQ ID NOS:2 and 85 and the second heavy chain comprises SEQ ID NOS:15 and 81;
(j) the first heavy chain comprises SEQ ID NOS:2 and 85 and the second heavy chain comprises SEQ ID NOS:15 and 82;
(k) the first heavy chain comprises SEQ ID NOS:2 and 85 and the second heavy chain comprises SEQ ID NOS:15 and 83; or
(l) the first heavy chain comprises SEQ ID NOS:2 and 85 and the second heavy chain comprises SEQ ID NOS:15 and 84.

In particular embodiments of this antibody:
(a) the first heavy chain comprises SEQ ID NOS:3 and 85 and the second heavy chain comprises SEQ ID NOS:15 and 86;
(b) the first heavy chain comprises SEQ ID NOS:3 and 85 and the second heavy chain comprises SEQ ID NOS:15 and 74;
(c) the first heavy chain comprises SEQ ID NOS:3 and 85 and the second heavy chain comprises SEQ ID NOS:15 and 75;
(d) the first heavy chain comprises SEQ ID NOS:3 and 85 and the second heavy chain comprises SEQ ID NOS:15 and 76;
(e) the first heavy chain comprises SEQ ID NOS:3 and 85 and the second heavy chain comprises SEQ ID NOS:15 and 77;
(f) the first heavy chain comprises SEQ ID NOS:3 and 85 and the second heavy chain comprises SEQ ID NOS:15 and 78;
(g) the first heavy chain comprises SEQ ID NOS:3 and 85 and the second heavy chain comprises SEQ ID NOS:15 and 79;
(h) the first heavy chain comprises SEQ ID NOS:3 and 85 and the second heavy chain comprises SEQ ID NOS:15 and 80;
(i) the first heavy chain comprises SEQ ID NOS:3 and 85 and the second heavy chain comprises SEQ ID NOS:15 and 81;
(j) the first heavy chain comprises SEQ ID NOS:3 and 85 and the second heavy chain comprises SEQ ID NOS:15 and 82;
(k) the first heavy chain comprises SEQ ID NOS:3 and 85 and the second heavy chain comprises SEQ ID NOS:15 and 83; or
(l) the first heavy chain comprises SEQ ID NOS:3 and 85 and the second heavy chain comprises SEQ ID NOS:15 and 84.

In certain other embodiments, the anti-HER2 antibody comprises a first heavy chain comprising a V_{H} and a Fc sequence each having at least 90% (e.g., 91%, 92%, 93%, 94%, 95%, 96%, 97%, 98%, 99%, or 100%) sequence identity to the amino acid sequences selected from the combinations in Table 5, i.e., any one of Combo #A-AJ, and a second heavy chain comprising a V_{H} and a Fc sequence each having at least 90% (e.g., 91%, 92%, 93%, 94%, 95%, 96%, 97%, 98%, 99%, or 100%) sequence identity to the amino acid sequences selected from the combinations in Table 6, i.e., any one of Combo #A-D. In any of these heavy chain combinations, the light chain polypeptide sequence has at least 90% (e.g., 91%, 92%, 93%, 94%, 95%, 96%, 97%, 98%, 99%, or 100%) sequence identity to an amino acid sequence selected from the group consisting of SEQ ID NOS:9-10 and 19.

### IV. Fc Polypeptides and Modifications Thereof

In some aspects, any of the antibodies described herein comprises an Fc polypeptide dimer in which either one or both Fc polypeptides in the dimer contain amino acid modifications relative to a wild-type Fc polypeptide. In some embodiments, the amino acid modifications in an Fc polypeptide (e.g., a modified Fc polypeptide) can result in binding of the Fc polypeptide dimer to a BBB receptor (e.g., a TfR), promote heterodimerization of the two Fc polypeptides in the dimer, modulate effector function, extend serum half-life, influence glycosylation, and/or reduce immunogenicity in humans. In some embodiments, the Fc polypeptides present in the antibody independently have an amino acid sequence identity of at least about 85%, 90%, 95%, 96%, 97%, 98%, or 99% to a corresponding wild-type Fc polypeptide (e.g., a human IgG1, IgG2, IgG3, or IgG4 Fc polypeptide). Examples and descriptions of modified Fc polypeptides (e.g., TfR-binding Fc polypeptides) can be found, e.g., in International Patent Publication No. WO 2018/152326, which is incorporated herein by reference in its entirety.

### Fc Polypeptide Modifications for BBB Receptor Binding

Provided herein are anti-HER2 antibodies that are capable of being transported across the BBB. Such a protein comprises a modified Fc polypeptide that binds to a BBB receptor. BBB receptors are expressed on BBB endothelia, as well as other cell and tissue types. In some embodiments, the BBB receptor is a TfR. A modified Fc polypeptide that binds to TfR is also referred to as having a TfR-binding site.

Amino acid residues designated in various Fc modifications, including those introduced in a modified Fc polypeptide that binds to a BBB receptor, e.g., TfR, are numbered herein using EU index numbering. Any Fc polypeptide, e.g., an IgG1, IgG2, IgG3, or IgG4 Fc polypeptide, may have modifications, e.g., amino acid substitutions, in one or more positions as described herein. In some embodiments, the domain that is modified for BBB (e.g., TfR) receptor-binding activity is a human Ig CH3 domain, such as an IgG1 CH3 domain. The CH3 domain can be of any IgG subtype, i.e., from IgG1, IgG2, IgG3, or IgG4. In the context of IgG1 antibodies, a CH3 domain refers to the segment of amino acids from about position 341 to about position 447 as numbered according to the EU numbering scheme.

In some embodiments, a modified Fc polypeptide that specifically binds to TfR binds to the apical domain of TfR and may bind to TfR without blocking or otherwise inhibiting binding of transferrin to TfR. In some embodiments, binding of transferrin to TfR is not substantially inhibited. In some embodiments, binding of transferrin to TfR is inhibited by less than about 50% (e.g., less than about 45%, 40%, 35%, 30%, 25%, 20%, 15%, 10%, or 5%).

In some embodiments, a BBB (e.g., TfR) receptor-binding Fc polypeptide present in an antibody described herein comprises one or more at least one, two, or three substitutions; and in some embodiments, at least four, five, six, seven, eight, nine, or ten substitutions at amino acid positions comprising 266, 267, 268, 269, 270, 271, 295, 297, 298, and 299, according to the EU numbering scheme. In some embodiments, a BBB (e.g., TfR) receptor-binding Fc polypeptide present in an antibody described herein comprises at least one, two, or three substitutions; and in some embodiments, at least four, five, six, seven, eight, or nine substitutions at amino acid positions comprising 274, 276, 283, 285, 286, 287, 288, 289, and 290, according to the EU numbering scheme. In some embodiments, a BBB (e.g., TfR) receptor-binding Fc polypeptide present in an antibody described herein comprises at least one, two, or three substitutions; and in some embodiments, at least four, five, six, seven, eight, nine, or ten substitutions at amino acid positions comprising 268, 269, 270, 271, 272, 292, 293, 294, 296, and 300, according to the EU numbering scheme. In some embodiments, a BBB (e.g., TfR) receptor-binding Fc polypeptide present in an antibody described herein comprises at least one, two, or three substitutions; and in some embodiments, at least four, five, six, seven, eight, or nine substitutions at amino acid positions comprising 272, 274, 276, 322, 324, 326, 329, 330, and 331, according to the EU numbering scheme. In some embodiments, a BBB (e.g., TfR) receptor-binding Fc polypeptide present in an antibody described herein comprises at least one, two, or three substitutions; and in some embodiments, at least four, five, six, or seven substitutions at amino acid positions comprising 345, 346, 347, 349, 437, 438, 439, and 440, according to the EU numbering scheme.

In some embodiments, a BBB (e.g., TfR) receptor-binding Fc polypeptide present in an antibody described herein comprises at least one, two, or three substitutions; and in some embodiments, at least four, five, six, seven, eight, or nine substitutions at amino acid positions 384, 386, 387, 388, 389, 390, 413, 416, and 421, according to the EU numbering scheme.

In some embodiments, a BBB (e.g., TfR) receptor-binding Fc polypeptide comprises at least one position having a substitution, relative to SEQ ID NO:95, as follows: Leu, Tyr, Met, or Val at position 384; Leu, Thr, His, or Pro at position 386; Val, Pro, or an acidic amino acid at position 387; an aromatic amino acid, e.g., Trp or Gly (e.g., Trp) at position 388; Val, Ser, or Ala at position 389; an acidic amino acid, Ala, Ser, Leu, Thr, or Pro at position 413; Thr or an acidic amino acid at position 416; or Trp, Tyr, His, or Phe at position 421. In some embodiments, a BBB (e.g., TfR) receptor-binding Fc polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set. Thus, for example, Ile may be present at position 384, 386, and/or position 413. In some embodiments, the acidic amino acid at position one, two, or each of positions 387, 413, and 416 is Glu. In other embodiments, the acidic amino acid at one, two or each of positions 387, 413, and 416 is Asp. In some embodiments, two, three, four five, six, seven, or all eight of positions 384, 386, 387, 388, 389, 413, 416, and 421 have an amino acid substitution as specified in this paragraph.

In some embodiments, a Fc polypeptide having modifications in amino acid positions 384, 386, 387, 388, 389, 390, 413, 416, and/or 421 comprises a native Asn at position 390. In some embodiments, the Fc polypeptide comprises Gly, His, Gln, Leu, Lys, Val, Phe, Ser, Ala, or Asp at position 390. In some embodiments, the Fc polypeptide further comprises one, two, three, or four substitutions at positions comprising 380, 391, 392, and 415. In some embodiments, Trp, Tyr, Leu, or Gln may be present at position 380. In some embodiments, Ser, Thr, Gln, or Phe may be present at position 391. In some embodiments, Gln, Phe, or His may be present at position 392. In some embodiments, Glu may be present at position 415.

In certain embodiments, the Fc polypeptide comprises two, three, four, five, six, seven, eight, nine, or ten positions selected from the following: Trp, Leu, or Glu at position 380; Tyr or Phe at position 384; Thr at position 386; Glu at position 387; Trp at position 388; Ser, Ala, Val, or Asn at position 389; Ser or Asn at position 390; Thr or Ser at position 413; Glu or Ser at position 415; Glu at position 416; and/or Phe at position 421. In some embodiments, the Fc polypeptide comprises all eleven positions as follows: Trp, Leu, or Glu at position 380; Tyr or Phe at position 384; Thr at position 386; Glu at position 387; Trp at position 388; Ser, Ala, Val, or Asn at position 389; Ser or Asn at position 390; Thr or Ser at position 413; Glu or Ser at position 415; Glu at position 416; and/or Phe at position 421.

In certain embodiments, the BBB (e.g., TfR) receptor-binding Fc polypeptide comprises Leu or Met at position 384; Leu, His, or Pro at position 386; Val at position 387; Trp at position 388; Val or Ala at position 389; Pro at position 413; Thr at position 416; and/or Trp at position 421. In some embodiments, the Fc polypeptide further comprises Ser, Thr, Gln, or Phe at position 391. In some embodiments, the Fc polypeptide further comprises Trp, Tyr, Leu, or Gln at position 380 and/or Gln, Phe, or His at position 392. In some embodiments, Trp is present at position 380 and/or Gln is present at position 392. In some embodiments, a BBB (e.g., TfR) receptor-binding Fc polypeptide does not have a Trp at position 380.

In other embodiments, a BBB (e.g., TfR) receptor-binding Fc polypeptide comprises Tyr at position 384; Thr at position 386; Glu or Val and position 387; Trp at position 388; Ser at position 389; Ser or Thr at position 413; Glu at position 416; and/or Phe at position 421. In some embodiments, the BBB (e.g., TfR) receptor-binding Fc polypeptide comprises a native Asn at position 390. In certain embodiments, the Fc polypeptide further comprises Trp, Tyr, Leu, or Gln at position 380; and/or Glu at position 415. In some embodiments, the Fc polypeptide further comprises Trp at position 380 and/or Glu at position 415.

In some embodiments, the BBB (e.g., TfR) receptor-binding Fc polypeptide comprises one or more of the following substitutions: Trp at position 380; Thr at position 386; Trp at position 388; Val at position 389; Ser or Thr at position 413; Glu at position 415; and/or Phe at position 421.

In additional embodiments, the BBB (e.g., TfR) receptor-binding Fc polypeptide further comprises one, two, or three positions selected from the following: position 414 is Lys, Arg, Gly, or Pro; position 424 is Ser, Thr, Glu, or Lys; and position 426 is Ser, Trp, or Gly.

In some embodiments, the BBB (e.g., TfR) receptor-binding Fc polypeptide has the sequence of SEQ ID NO:97. In some embodiments of the antibodies described herein, one of the two Fc polypeptides in the Fc polypeptide dimer can be a BBB (e.g., TfR) receptor-binding Fc polypeptide having the sequence of SEQ ID NO:97, while the other Fc polypeptide in the Fc polypeptide dimer can have the sequence of a wild-type Fc polypeptide (e.g., SEQ ID NO:95). In other embodiments of the antibodies described herein, both Fc polypeptides in the Fc polypeptide dimer can be a BBB (e.g., TfR) receptor-binding Fc polypeptide having the sequence of SEQ ID NO:97.

In some embodiments of the antibodies described herein, the first Fc polypeptide and/or the second Fc polypeptide independently comprises Tyr at position 384, Thr at position 386, Glu at position 387, Trp at position 388, Ser at position 389, Ser at position 413, Glu at position 415, Glu at position 416, and Phe at position 421, according to EU numbering, and a sequence having at least 90% (e.g., 91%, 92%, 93%, 94%, 95%, 96%, 97%, 98%, 99%, or 100%) identity to a sequence selected from SEQ ID NOS:74-84, 86, and 97-101.

In some embodiments of the antibodies described herein, one of the two Fc polypeptides in the Fc polypeptide dimer can be a BBB (e.g., TfR) receptor-binding Fc polypeptide comprising Tyr at position 384, Thr at position 386, Glu at position 387, Trp at position 388, Ser at position 389, Ser at position 413, Glu at position 415, Glu at position 416, and Phe at position 421, according to EU numbering, and a sequence having at least 90% identity to the sequence of SEQ ID NO:97, while the other Fc polypeptide in the Fc polypeptide dimer can have the sequence of a wild-type Fc polypeptide (e.g., SEQ ID NO:95).

In some embodiments of the antibodies described herein, the first Fc polypeptide and/or the second Fc polypeptide independently comprises Tyr at position 384, Thr at position 386, Glu at position 387, Trp at position 388, Ala at position 389, Thr at position 413, Glu at position 415, Glu at position 416, and Phe at position 421, according to EU numbering, and a sequence having at least 90% (e.g., 91%, 92%, 93%, 94%, 95%, 96%, 97%, 98%, 99%, or 100%) identity to a sequence selected from SEQ ID NOS:101-105.

In some embodiments of the antibodies described herein, one of the two Fc polypeptides in the Fc polypeptide dimer can be a BBB (e.g., TfR) receptor-binding Fc polypeptide comprising Tyr at position 384, Thr at position 386, Glu at position 387, Trp at position 388, Ala at position 389, Thr at position 413, Glu at position 415, Glu at position 416, and Phe at position 421, according to EU numbering, and a sequence having at least 90% identity to the sequence of SEQ ID NO:101, while the other Fc polypeptide in the Fc polypeptide dimer can have the sequence of a wild-type Fc polypeptide (e.g., SEQ ID NO:95).

In some embodiments, the BBB (e.g., TfR) receptor-binding Fc polypeptide has the sequence of SEQ ID NO:101. In some embodiments of the antibodies described herein, one of the two Fc polypeptides in the Fc polypeptide dimer can be a BBB (e.g., TfR) receptor-binding Fc polypeptide having the sequence of SEQ ID NO:101, while the other Fc polypeptide in the Fc polypeptide dimer can have the sequence of a wild-type Fc polypeptide (e.g., SEQ ID NO:95). In other embodiments of the antibodies described herein, both Fc polypeptides in the Fc polypeptide dimer can be a BBB (e.g., TfR) receptor-binding Fc polypeptide having the sequence of SEQ ID NO:101.

In some embodiments, the BBB (e.g., TfR) receptor-binding Fc polypeptide comprises the following substitutions listed in Table A below (according to EU numbering):

TABLE A

| | 378 | 379 | 380 | 381 | 382 | 383 | 384 | 385 | 386 | 387 | 388 | 389 | 390 | 391 |
|---|---|---|---|---|---|---|---|---|---|---|---|---|---|---|
| Wild-type | A | V | E | W | E | S | N | G | Q | P | E | N | N | Y |
| CH3C.35.20.1 | . | . | . | . | . | . | F | . | T | E | W | S | S | . |
| CH3C.35.20.2 | . | . | . | . | . | . | Y | . | T | E | W | A | S | . |
| CH3C.35.20.3 | . | . | . | . | . | . | Y | . | T | E | W | V | S | . |
| CH3C.35.20.4 | . | . | . | . | . | . | Y | . | T | E | W | S | S | . |
| CH3C.35.20.5 | . | . | . | . | . | . | F | . | T | E | W | A | S | . |
| CH3C.35.20.6 | . | . | . | . | . | . | F | . | T | E | W | V | S | . |
| CH3C.35.2.a.1 | . | . | W | . | . | . | F | . | T | E | W | S | S | . |
| CH3C.35.2.a.2 | . | . | W | . | . | . | Y | . | T | E | W | A | S | . |
| CH3C.35.2.a.3 | . | . | W | . | . | . | Y | . | T | E | W | V | S | . |
| CH3C.35.2.a.4 | . | . | W | . | . | . | Y | . | T | E | W | S | S | . |
| CH3C.35.2.a.5 | . | . | W | . | . | . | F | . | T | E | W | A | S | . |
| CH3C.35.2.a.6 | . | . | W | . | . | . | F | . | T | E | W | V | S | . |
| CH3C.35.23.1 | . | . | . | . | . | . | F | . | T | E | W | S | . | . |
| CH3C.35.23.2 | . | . | . | . | . | . | Y | . | T | E | W | A | . | . |
| CH3C.35.23.3 | . | . | . | . | . | . | Y | . | T | E | W | V | . | . |
| CH3C.35.23.4 | . | . | . | . | . | . | Y | . | T | E | W | S | . | . |
| CH3C.35.23.5 | . | . | . | . | . | . | F | . | T | E | W | A | . | . |
| CH3C.35.23.6 | . | . | . | . | . | . | F | . | T | E | W | V | . | . |
| CH3C.35.24.1 | . | . | W | . | . | . | F | . | T | E | W | S | . | . |
| CH3C.35.24.2 | . | . | W | . | . | . | Y | . | T | E | W | A | . | . |
| CH3C.35.24.3 | . | . | W | . | . | . | Y | . | T | E | W | V | . | . |
| CH3C.35.24.4 | . | . | W | . | . | . | Y | . | T | E | W | S | . | . |
| CH3C.35.24.5 | . | . | W | . | . | . | F | . | T | E | W | A | . | . |
| CH3C.35.24.6 | . | . | W | . | . | . | F | . | T | E | W | V | . | . |
| CH3C.35.21.17.1 | . | . | L | . | . | . | F | . | T | E | W | S | S | . |
| CH3C.35.21.17.2 | . | . | L | . | . | . | Y | . | T | E | W | A | S | . |
| CH3C.35.21.17.3 | . | . | L | . | . | . | Y | . | T | E | W | V | S | . |
| CH3C.35.21.17.4 | . | . | L | . | . | . | Y | . | T | E | W | S | S | . |
| CH3C.35.21.17.5 | . | . | L | . | . | . | F | . | T | E | W | A | S | . |
| CH3C.35.21.17.6 | . | . | L | . | . | . | F | . | T | E | W | V | S | . |
| CH3C.35.20 | . | . | . | . | . | . | Y | . | T | E | W | S | S | . |
| CH3C.35.21 | . | . | W | . | . | . | Y | . | T | E | W | S | S | . |
| CH3C.35.22 | . | . | W | . | . | . | Y | . | T | E | W | S | . | . |
| CH3C.35.23 | . | . | . | . | . | . | Y | . | T | E | W | S | . | . |
| CH3C.35.24 | . | . | W | . | . | . | Y | . | T | E | W | S | . | . |
| CH3C.35.21.17 | . | . | L | . | . | . | Y | . | T | E | W | S | S | . |
| CH3C.35.N390 | . | . | . | . | . | . | Y | . | T | E | W | S | . | . |
| CH3C.35.20.1.1 | | | | | | | F | | T | E | W | S | S | |
| CH3C.35.23.2.1 | | | | | | | Y | | T | E | W | A | | |
| CH3C.35.23.1.1 | | | | | | | F | | T | E | W | S | | |
| CH3C.35.S413 | | | | | | | Y | | T | E | W | S | S | |
| CH3C.35.23.3.1 | | | | | | | Y | | T | E | W | V | | |
| CH3C.35.N390.1 | | | | | | | Y | | T | E | W | S | | |
| CH3C.35.23.6.1 | | | | | | | F | | T | E | W | V | | |
| | 392 | 411 | 412 | 413 | 414 | 415 | 416 | 417 | 418 | 419 | 420 | 421 | 422 | 423 |
| Wild-type | K | T | V | D | K | S | R | W | Q | Q | G | N | V | F |
| CH3C.35.20.1 | . | . | . | T | . | E | E | . | . | . | . | F | . | . |
| CH3C.35.20.2 | . | . | . | T | . | E | E | . | . | . | . | F | . | . |
| CH3C.35.20.3 | . | . | . | T | . | E | E | . | . | . | . | F | . | . |
| CH3C.35.20.4 | . | . | . | S | . | E | E | . | . | . | . | F | . | . |
| CH3C.35.20.5 | . | . | . | T | . | E | E | . | . | . | . | F | . | . |
| CH3C.35.20.6 | . | . | . | T | . | E | E | . | . | . | . | F | . | . |
| CH3C.35.2.a.1 | . | . | . | T | . | E | E | . | . | . | . | F | . | . |
| CH3C.35.2.a.2 | . | . | . | T | . | E | E | . | . | . | . | F | . | . |
| CH3C.35.2.a.3 | . | . | . | T | . | E | E | . | . | . | . | F | . | . |
| CH3C.35.2.a.4 | . | . | . | S | . | E | E | . | . | . | . | F | . | . |
| CH3C.35.2.a.5 | . | . | . | T | . | E | E | . | . | . | . | F | . | . |
| CH3C.35.2.a.6 | . | . | . | T | . | E | E | . | . | . | . | F | . | . |
| CH3C.35.23.1 | . | . | . | T | . | E | E | . | . | . | . | F | . | . |
| CH3C.35.23.2 | . | . | . | T | . | E | E | . | . | . | . | F | . | . |
| CH3C.35.23.3 | . | . | . | T | . | E | E | . | . | . | . | F | . | . |
| CH3C.35.23.4 | . | . | . | S | . | E | E | . | . | . | . | F | . | . |
| CH3C.35.23.5 | . | . | . | T | . | E | E | . | . | . | . | F | . | . |
| CH3C.35.23.6 | . | . | . | T | . | E | E | . | . | . | . | F | . | . |
| CH3C.35.24.1 | . | . | . | T | . | E | E | . | . | . | . | F | . | . |
| CH3C.35.24.2 | . | . | . | T | . | E | E | . | . | . | . | F | . | . |
| CH3C.35.24.3 | . | . | . | T | . | E | E | . | . | . | . | F | . | . |
| CH3C.35.24.4 | . | . | . | S | . | E | E | . | . | . | . | F | . | . |
| CH3C.35.24.5 | . | . | . | T | . | E | E | . | . | . | . | F | . | . |
| CH3C.35.24.6 | . | . | . | T | . | E | E | . | . | . | . | F | . | . |
| CH3C.35.21.17.1 | . | . | . | T | . | E | E | . | . | . | . | F | . | . |
| CH3C.35.21.17.2 | . | . | . | T | . | E | E | . | . | . | . | F | . | . |
| CH3C.35.21.17.3 | . | . | . | T | . | E | E | . | . | . | . | F | . | . |
| CH3C.35.21.17.4 | . | . | . | S | . | E | E | . | . | . | . | F | . | . |
| CH3C.35.21.17.5 | . | . | . | T | . | E | E | . | . | . | . | F | . | . |
| CH3C.35.21.17.6 | . | . | . | T | . | E | E | . | . | . | . | F | . | . |
| CH3C.35.20 | . | . | . | T | . | E | E | . | . | . | . | F | . | . |
| CH3C.35.21 | . | . | . | T | . | E | E | . | . | . | . | F | . | . |
| CH3C.35.22 | . | . | . | T | . | . | E | . | . | . | . | F | . | . |
| CH3C.35.23 | . | . | . | T | . | E | E | . | . | . | . | F | . | . |
| CH3C.35.24 | . | . | . | T | . | E | E | . | . | . | . | F | . | . |
| CH3C.35.21.17 | . | . | . | T | . | E | E | . | . | . | . | F | . | . |
| CH3C.35.N390 | . | . | . | T | . | . | E | . | . | . | . | F | . | . |
| CH3C.35.20.1.1 | | | | S | | E | E | | | | | F | | |
| CH3C.35.23.2.1 | | | | S | | | E | | | | | F | | |
| CH3C.35.23.1.1 | | | | S | | E | E | | | | | F | | |
| CH3C.35.S413 | | | | S | | | E | | | | | F | | |
| CH3C.35.23.3.1 | | | | S | | E | E | | | | | F | | |
| CH3C.35.N390.1 | | | | S | | | E | | | | | F | | |
| CH3C.35.23.6.1 | | | | S | | E | E | | | | | F | | |

### Fc Polypeptide Modifications for Heterodimerization

In some embodiments, the Fc polypeptides present in any antibody described herein include knob and hole mutations to promote heterodimer formation and hinder homodimer formation. Generally, the modifications introduce a protuberance (“knob”) at the interface of a first polypeptide and a corresponding cavity (“hole”) in the interface of a second polypeptide, such that the protuberance can be positioned in the cavity so as to promote heterodimer formation and thus hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine). In some embodiments, such additional mutations are at a position in the Fc polypeptide that does not have a negative effect on binding of the polypeptide to a BBB receptor, e.g., TfR.

In one illustrative embodiment of a knob and hole approach for dimerization, position 366 (numbered according to the EU numbering scheme) of one of the Fc polypeptides present in the antibody comprises a tryptophan in place of a native threonine. The other Fc polypeptide in the dimer has a valine at position 407 (numbered according to the EU numbering scheme) in place of the native tyrosine. The other Fc polypeptide may further comprise a substitution in which the native threonine at position 366 (numbered according to the EU numbering scheme) is substituted with a serine and a native leucine at position 368 (numbered according to the EU numbering scheme) is substituted with an alanine. Thus, one of the Fc polypeptides of an antibody described herein has the T366W knob mutation and the other Fc polypeptide has the Y407V mutation, which is typically accompanied by the T366S and L368A hole mutations.

In some embodiments, one or both Fc polypeptides present in an antibody described herein may also be engineered to contain other modifications for heterodimerization, e.g., electrostatic engineering of contact residues within a CH3-CH3 interface that are naturally charged or hydrophobic patch modifications.

For example, in some embodiments, an antibody described herein can contain an Fc polypeptide dimer that has one Fc polypeptide having the T366W knob mutation and at least 90% (e.g., 91%, 92%, 93%, 94%, 95%, 96%, 97%, 98%, or 99%) identity to the sequence of SEQ ID NO:107 and the other Fc polypeptide having the T366S, L368A, and Y407V hole mutations and at least 90% identity to the sequence of SEQ ID NO:85. In certain embodiments, one or both Fc polypeptides in the Fc polypeptide dimer can be a TfR-binding Fc polypeptide. In particular embodiments, an antibody described herein can contain an Fc polypeptide dimer that has (i) a first Fc polypeptide having the sequence of SEQ ID NO:85, and (ii) a second Fc polypeptide having the sequence of SEQ ID NO:98. In particular embodiments, an antibody described herein can contain an Fc polypeptide dimer that has (i) a first Fc polypeptide having the sequence of SEQ ID NO:85, and (ii) a second Fc polypeptide having the sequence of SEQ ID NO:102.

### Fc Polypeptide Modifications for Modulating Effector Function

In some embodiments, one or both Fc polypeptides present in any antibody described herein may comprise modifications that reduce TfR-mediated effector function upon TfR binding, i.e., having a reduced ability to induce certain biological functions upon binding to an Fc receptor expressed on an effector cell that mediates the effector function. Examples of antibody effector functions include, but are not limited to, Clq binding and complement dependent cytotoxicity (CDC), Fc receptor binding, antibody-dependent cell-mediated cytotoxicity (ADCC), antibody-dependent cell-mediated phagocytosis (ADCP), down-regulation of cell surface receptors (e.g., B cell receptor), and B-cell activation. Effector functions may vary with the antibody class. For example, native human IgG1 and IgG3 antibodies can elicit ADCC and CDC activities upon binding to an appropriate Fc receptor present on an immune system cell; and native human IgG1, IgG2, IgG3, and IgG4 can elicit ADCP functions upon binding to the appropriate Fc receptor present on an immune cell.

In some embodiments, one or both Fc polypeptides present in an antibody described herein may comprise modifications that reduce or eliminate TfR-mediated effector function. Illustrative Fc polypeptide mutations that reduce TfR-mediated effector function include, but are not limited to, substitutions in a CH2 domain, e.g., at positions 234 and 235, according to the EU numbering scheme. For example, in some embodiments, one or both Fc polypeptides can comprise alanine residues at positions 234 and 235. Thus, one or both Fc polypeptides may have L234A and L235A (also referred to as “LALA” herein) substitutions.

Additional Fc polypeptide mutations that modulate an effector function include, but are not limited to, the following: position 329 may have a mutation in which proline is substituted with a glycine, alanine, serine, or arginine or an amino acid residue large enough to destroy the Fc/Fcγ receptor interface that is formed between proline 329 of the Fc and tryptophan residues Trp 87 and Trp 110 of FcγRIII. Additional illustrative substitutions include S228P, E233P, L235E, N297A, N297D, and P331S, according to the EU numbering scheme. Multiple substitutions may also be present, e.g., L234A and L235A of a human IgG1 Fc region; L234A, L235A, and P329G of a human IgG1 Fc region; S228P and L235E of a human IgG4 Fc region; L234A and G237A of a human IgG1 Fc region; L234A, L235A, and G237A of a human IgG1 Fc region; V234A and G237A of a human IgG2 Fc region; L235A, G237A, and E318A of a human IgG4 Fc region; and S228P and L236E of a human IgG4 Fc region, according to the EU numbering scheme. In some embodiments, one or both Fc polypeptides may have one or more amino acid substitutions that modulate ADCC, e.g., substitutions at positions 298, 333, and/or 334, according to the EU numbering scheme. In some embodiments, one or both Fc polypeptides may have L234A, L235A, and P329G or P329S substitutions, according to the EU numbering scheme.

In some embodiments, one or both Fc polypeptides present in an antibody described herein may comprise modifications that are capable of enhancing HER2-mediated effector function upon HER2 binding, i.e., enhancing the ability to induce certain biological functions upon binding to an Fc receptor expressed on an effector cell that mediates the effector function. Examples of antibody effector functions are described above. Illustrative Fc polypeptide mutations that are capable of enhancing HER2-mediated effector function include, but are not limited to, substitutions in a CH2 domain, e.g., at positions 239 and/or 332, according to the EU numbering scheme. For example, in some embodiments, one or both Fc polypeptides can comprise aspartic acid at position 239 and/or glutamic acid at position 332. Thus, one or both Fc polypeptides may have a S239D and/or a I332E substitution, according to EU numbering.

“cis-LALA” Configuration

In some embodiments of any antibody described herein, only one of the two Fc polypeptides (but not both Fc polypeptides) of the two Fc polypeptides in the antibody is modified to reduce TfR-mediated effector function upon TfR binding. The other Fc polypeptide does not contain a TfR-binding site or any modifications that reduce effector function. The Fc polypeptide dimer in the antibody that has only one of the two Fc polypeptides containing both the TfR-binding site and modifications that reduce FcγR binding (e.g., LALA substitutions) when bound to TfR, while the other Fc polypeptide does not contain a TfR-binding site or any modifications that reduce FcγR binding, is referred to as having the cis-LALA configuration.

For example, in some embodiments, an antibody described herein can contain an Fc polypeptide dimer having the cis-LALA configuration that has (i) a first Fc polypeptide having the sequence of SEQ ID NO:86, which has both a TfR-binding site and LALA substitutions, as well as a knob modification, and (ii) a second Fc polypeptide having at least 90% identity to the sequence of SEQ ID NO:85, which only has a hole modification. In some embodiments, an antibody described herein can contain an Fc polypeptide dimer having the cis-LALA configuration that has (i) a first Fc polypeptide having the sequence of SEQ ID NO:103, which has both a TfR-binding site and LALA substitutions, as well as a knob modification, and (ii) a second Fc polypeptide having at least 90% identity to the sequence of SEQ ID NO:85, which only has a hole modification.

In particular embodiments, an antibody described herein can contain an Fc polypeptide dimer having the cis-LALA configuration that has (i) a first Fc polypeptide comprising Ala at position 234, Ala at position 235, Trp at position 366, Tyr at position 384, Thr at position 386, Glu at position 387, Trp at position 388, Ser at position 389, Ser at position 413, Glu at position 415, Glu at position 416, and Phe at position 421, according to EU numbering, and a sequence having at least 90% identity to the sequence of SEQ ID NO:86, and (ii) a second Fc polypeptide comprising Ser at position 366, Ala at position 368, and Val at position 407, according to EU numbering, and a sequence having at least 90% identity to the sequence of SEQ ID NO:85.

In particular embodiments, an antibody described herein can contain an Fc polypeptide dimer having the cis-LALA configuration that has (i) a first Fc polypeptide comprises Ser at position 366, Ala at position 368, and Val at position 407, according to EU numbering, and a sequence having at least 90% identity to the sequence of SEQ ID NO:85, and (ii) a second Fc polypeptide comprises Ala at position 234, Ala at position 235, Trp at position 366, Tyr at position 384, Thr at position 386, Glu at position 387, Trp at position 388, Ser at position 389, Ser at position 413, Glu at position 415, Glu at position 416, and Phe at position 421, according to EU numbering, and a sequence having at least 90% identity to the sequence of SEQ ID NO:86.

In particular embodiments, an antibody described herein can contain an Fc polypeptide dimer having the cis-LALA configuration that has (i) a first Fc polypeptide comprising Ala at position 234, Ala at position 235, Trp at position 366, Tyr at position 384, Thr at position 386, Glu at position 387, Trp at position 388, Ala at position 389, Thr at position 413, Glu at position 415, Glu at position 416, and Phe at position 421, according to EU numbering, and a sequence having at least 90% identity to the sequence of SEQ ID NO:103, and (ii) a second Fc polypeptide comprising Ser at position 366, Ala at position 368, and Val at position 407, according to EU numbering, and a sequence having at least 90% identity to the sequence of SEQ ID NO:85.

In particular embodiments, an antibody described herein can contain an Fc polypeptide dimer having the cis-LALA configuration that has (i) a first Fc polypeptide comprising Ser at position 366, Ala at position 368, and Val at position 407, according to EU numbering, and a sequence having at least 90% identity to the sequence of SEQ ID NO:85, and (ii) a second Fc polypeptide comprising Ala at position 234, Ala at position 235, Trp at position 366, Tyr at position 384, Thr at position 386, Glu at position 387, Trp at position 388, Ala at position 389, Thr at position 413, Glu at position 415, Glu at position 416, and Phe at position 421, according to EU numbering, and a sequence having at least 90% identity to the sequence of SEQ ID NO:103.

### Fc Polypeptide Modifications for Extending Serum Half-Life

In some embodiments, modifications to enhance serum half-life may be introduced into any antibody described herein. For example, in some embodiments, one or both Fc polypeptides present in an antibody described herein may comprise a tyrosine at position 252, a threonine at position 254, and a glutamic acid at position 256, as numbered according to the EU numbering scheme. Thus, one or both Fc polypeptides may have M252Y, S254T, and T256E substitutions. Alternatively, one or both Fc polypeptides may have M428L and N434S substitutions, as numbered according to the EU numbering scheme. Alternatively, one or both Fc polypeptides may have an N434S or N434A substitution.

Fc Polypeptide with C-terminal Lysine Residue Removed

In some embodiments of the antibodies described herein, one or both of the Fc polypeptides can have its C-terminal lysine removed (e.g., the Lys residue at position 447 of the Fc polypeptide, according to EU numbering). The C-terminal lysine residue is highly conserved in immunoglobulins across many species and may be fully or partially removed by the cellular machinery during protein production. In some embodiments, removal of the C-terminal lysines in the Fc polypeptides can improve the stability of the antibodies.

### V. Preparation of Antibodies

For preparing an antibody described herein, many techniques known in the art can be used. In some embodiments, the genes encoding the heavy and light chains of an antibody of interest can be cloned from a cell, e.g, from a hybridoma. Gene libraries encoding heavy and light chains of monoclonal antibodies can also be made from hybridoma or plasma cells. Alternatively, phage or yeast display technology can be used to identify antibodies and Fab fragments that specifically bind to selected antigens.

Antibodies can be produced using any number of expression systems, including prokaryotic and eukaryotic expression systems. In some embodiments, the expression system is a mammalian cell expression system, such as a hybridoma, or a CHO cell expression system. Many such systems are widely available from commercial suppliers. In some embodiments, the polynucleotides encoding the polypeptides that comprise the antibody may be expressed using a single vector, e.g., in a di-cistronic expression unit, or under the control of different promoters. In other embodiments, the polynucleotides encoding the polypeptides that comprise the antibody may be expressed using separate vectors.

In some aspects, the disclosure provides isolated nucleic acids comprising a nucleic acid sequence encoding any of the polypeptides comprising the antibodies as described herein, vectors comprising such nucleic acids, and host cells into which the nucleic acids are introduced that are used to replicate the nucleic acids and/or to express the antibodies.

In some embodiments, a polynucleotide (e.g., an isolated polynucleotide) comprises a nucleotide sequence encoding a polypeptide that comprises the antibody as disclosed herein (e.g., as described in Section III above). In some embodiments, the polynucleotide comprises a nucleotide sequence encoding one or more amino acid sequences (e.g., heavy chain, light chain, and/or Fc polypeptide sequences) disclosed in the Informal Sequence Listing below. In some embodiments, the polynucleotide comprises a nucleotide sequence encoding an amino acid sequence having at least 85% sequence identity (e.g., at least 85%, at least 90%, at least 91%, at least 92%, at least 93%, at least 94%, at least 95%, at least 96%, at least 97%, at least 98%, or at least 99% sequence identity) to a sequence disclosed in the Informal Sequence Listing below. In some embodiments, a polynucleotide as described herein is operably linked to a heterologous nucleic acid, e.g., a heterologous promoter.

Suitable vectors containing polynucleotides encoding antibodies of the present disclosure, or fragments thereof, include cloning vectors and expression vectors. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a nucleic acid of the present disclosure. The expression vector may replicate in the host cells either as episomes or as an integral part of the chromosomal DNA. Suitable expression vectors include but are not limited to plasmids, viral vectors, including adenoviruses, adeno-associated viruses, retroviruses, and any other vector.

Suitable host cells for cloning or expressing a polynucleotide or vector as described herein include prokaryotic or eukaryotic cells. In some embodiments, the host cell is prokaryotic. In some embodiments, the host cell is eukaryotic, e.g., Chinese Hamster Ovary (CHO) cells or lymphoid cells. In some embodiments, the host cell is a human cell, e.g., a Human Embryonic Kidney (HEK) cell.

In another aspect, methods of making an antibody as described herein are provided. In some embodiments, the method includes culturing a host cell as described herein (e.g., a host cell expressing a polynucleotide or vector as described herein) under conditions suitable for expression of the antibody. In some embodiments, the antibody is subsequently recovered from the host cell (or host cell culture medium). In some embodiments, the antibody is purified, e.g., by chromatography.

### VI. Therapeutic Methods

In some aspects, provided herein are methods for treating a cancer (e.g., a HER2-positive cancer) or treating brain metastasis of a cancer (e.g., a HER2-positive cancer) in a subject by administering to the subject a therapeutically effective amount of an antibody described herein or a pharmaceutical composition thereof. Also provided herein are methods of transcytosis of an antibody variable region that is capable of binding HER2 (e.g., human HER2), or an antigen-binding fragment thereof, across an endothelium. In some embodiments, the methods comprise contacting the endothelium with a composition comprising an antibody described herein. In some embodiments, the endothelium is the blood brain barrier (BBB).

Non-limiting examples of HER2-positive cancers that can be treated according to the methods provided herein include HER2-positive breast, ovarian, bladder, salivary gland, endometrial, pancreatic, and non-small-cell lung cancer (NSCLC), as well as HER2-positive gastric adenocarcinoma and/or a HER2-positive gastroesophageal junction adnocarcinoma. In some embodiments, the HER2-positive cancer is a HER2-positive breast cancer. In some embodiments, the HER2-positive cancer is a HER2-positive gastric adenocarcinoma and/or a HER2-positive gastroesophageal junction adnocarcinoma. In some embodiments, the HER2-positive cancer is a metastatic cancer.

In still other aspects, provided herein are methods for treating metastasis of a cancer (e.g., a HER2-positive cancer). In some embodiments, the methods comprise administering to the subject a therapeutically effective amount of an antibody described herein. In some embodiments, the metastasis is a brain metastasis of a HER2-positive cancer described above. In some embodiments, the metastasis is a brain meatstasis of a HER2-positive breast cancer. In some embodiments, the metastasis is a brain metastasis of a HER2-positive gastric adenocarcinoma and/or a HER2-positive gastroesophageal junction adnocarcinoma.

In some embodiments, the therapeutic benefit can comprise a decrease in or slowing of tumor growth, a decrease in tumor size (e.g., volume), a decrease in tumor cell viability, a decrease in the number of metastatic lesions, amelioration in one or more signs or symptoms of a cancer (e.g., HER2-positive cancer), and/or an increase in patient survival. In some embodiments, tumor cell survival, tumor growth, tumor size, and/or the number of metastatic lesions is decreased by at least about 10%, 20%, 30%, 40%, 50%, 60%, 70%, 80%, 90%, or more.

In some embodiments, the antibody antagonizes HER2 activity. In some embodiments, HER2 activity is inhibited (e.g., by at least about 10%, 20%, 30%, 40%, 50%, 60%, 70%, 80%, 90%, or more).

The route of administration of an an antibody described herein can be oral, intraperitoneal, transdermal, subcutaneous, intravenous, intramuscular, intrathecal, inhalational, topical, intralesional, rectal, intrabronchial, nasal, transmucosal, intestinal, ocular or otic delivery, or any other methods known in the art. In some embodiments, the antibody is administered orally, intravenously, or intraperitoneally.

### VII. Pharmaceutical Compositions and Kits

In other aspects, pharmaceutical compositions and kits comprising an antibody in accordance with the disclosure are provided.

### Pharmaceutical Compositions

Guidance for preparing formulations for use in the disclosure can be found in any number of handbooks for pharmaceutical preparation and formulation that are known to those of skill in the art.

In some embodiments, a pharmaceutical composition comprises an antibody as described herein and further comprises one or more pharmaceutically acceptable carriers and/or excipients. A pharmaceutically acceptable carrier includes any solvents, dispersion media, or coatings that are physiologically compatible and that do not interfere with or otherwise inhibit the activity of the active agent.

In some embodiments, the antibody can be formulated for parenteral administration by injection. Typically, a pharmaceutical composition for use in in vivo administration is sterile, e.g., heat sterilization, steam sterilization, sterile filtration, or irradiation.

Dosages and desired drug concentration of pharmaceutical compositions described herein may vary depending on the particular use envisioned.

### Kits

In some embodiments, a kit for use in treating a cancer (e.g., a HER2-positive cancer) comprising an antibody described herein is provided. In some embodiments, the kit further comprises one or more additional therapeutic agents. For example, in some embodiments, the kit comprises an antibody as described herein and further comprises one or more additional therapeutic agents for use in the treatment of cancer. In some embodiments, the kit further comprises instructional materials containing directions (i.e., protocols) for the practice of the methods described herein (e.g., instructions for using the kit for administering an antibody). While the instructional materials typically comprise written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD-ROM), and the like. Such media may include addresses to internet sites that provide such instructional materials.

### VIII. Examples

The present invention will be described in greater detail by way of specific examples. The following examples are offered for illustrative purposes only, and are not intended to limit the invention in any manner.

### Example 1. Generation of Anti-HER2 Bispecific Antibodies

### Expression and Purification of Recombinant Bispecific Antibody Variants

Expression plasmids consisting of (i) a heavy chain polypeptide comprising a TfR-binding site and a knob (T366W) mutation, (ii) a heavy chain polypeptide comprising hole (T366S/L368A/Y407V) mutations, and (iii) light chains according to the combinations in Table 2 are co-transfected in Expi293 or ExpiCHO cells. Recombinant bispecific antibody variants are subsequently purified from conditioned media by loading supernatant over a protein A column (GE Mab Select SuRe). The column is washed with 10 column volumes of PBS, pH 7.4. The proteins are eluted with 50 mM sodium citrate, pH 3.0 containing 150 mM NaCl, and immediately neutralized with 200 mM arginine, 137 mM succinic acid, pH 5.0. The proteins are further purified by size-exclusion chromatography (GE Superdex200) using 200 mM arginine, 137 mM succinic acid, pH 5.0 as running buffer. The purified proteins are confirmed by intact mass LC/MS, and purity of >95% is confirmed by SDS-PAGE and analytical HPLC-SEC.

The heavy chain polypeptides may be further processed during cell culture production, such that the C-terminal lysine residue is removed. Thus, the bispecific antibodies listed in Table 2 may refer to protein molecules comprising heavy chains that are unprocessed (i.e., comprise the C-terminal lysine residue); protein molecules comprising one or more heavy chains that are processed (i.e., the C-terminal lysine residue is absent); or a mixture of protein molecules having processed and/or unprocessed heavy chains.

### Example 2. Biacore Assessment of Anti-HER2 Antibodies

HER2 extracellular domain (ECD) binding affinities of engineered anti-HER2 antibodies were measured by SPR using a Biacore 8K instrument. Antibodies were captured on Biacore™ Series S CM5 sensor chips immobilized with mouse anti-human Fab (human Fab capture kit from GE Healthcare) followed by injections of serial 3-fold dilutions of recombinant HER2 ECD at a flow rate of 30 μL/min. Each sample was analyzed using a 3-minute association followed by a 10-minute dissociation. After each injection, the sensor chip was regenerated using a 50 mM glycine pH2.0 regeneration buffer. A 1:1 Languir model of simultaneous fitting of kₒₙ and k_{off} was used for kinetics analysis.

The consensus sequence for Anti-HER2_D4 light chain control (SEQ ID NO:87) and Anti-HER2_D2 light chain control (SEQ ID NO:94) was analyzed. Structural analysis suggested that a Y residue at position 91 was involved in the structural organization of CDR loops for efficient HER2 D4 binding while an H residue at the same position was less involved in HER2 D2 binding. After screening of single amino acid substitutions at position 91, Y and F residues at this position were selected for further testing.

Affinity matured anti-HER2 light chain sequences (SEQ ID NOS:9 and 10) were paired with anti-HER2_D2 heavy chain control (SEQ ID NO:92) and anti-HER2_D4 heavy chain control (SEQ ID NO:93) for HER2 binding K_{D} measurement. Results are shown in Table 7.

TABLE 7

| | | HER2 binding |
|---|---|---|
| LC | HC | K_{D} (nM) |
| Anti-HER2_D4 light chain | Anti-HER2_D2 | 41 |
| control (SEQ ID NO: 87) | heavy chain | |
| SEQ ID NO: 10 | control | 14 |
| SEQ ID NO: 9 | (SEQ ID NO: 92) | 13 |
| Anti-HER2_D2 light chain | | 2.9 |
| control (SEQ ID NO: 94) | | |
| Anti-HER2_D4 light chain | Anti-HER2_D4 | 3 |
| control (SEQ ID NO: 87) | heavy chain | |
| SEQ ID NO: 10 | control | 1.7 |
| SEQ ID NO: 9 | (SEQ ID NO: 93) | 1.5 |
| Anti-HER2_D2 light chain | | No binding |
| control (SEQ ID NO: 94) | | |

SEQ ID NO:9 and 10 light chains showed HER2 binding when paired with both Anti-HER2 D2 and D4 heavy chain controls. SEQ ID NO:9 and 10 light chains showed lower HER2 binding affinity (13 K_{D} and 14 K_{D} respectively) compared to Anti-HER2_D2 light chain control (2.9 K_{D}) when paired with Anti-HER2_D2 heavy chain control. In contrast, SEQ ID NO:9 and 10 light chains showed higher HER2 binding affinity (1.5 K_{D} and 1.7 K_{D} respectively) compared to Anti-HER2_D4 light chain control (3 K_{D}) when paired with Anti-HER2_D4 heavy chain control.

Thirteen amino acid positions in CDR H1, H2, or H3 of Anti-HER2_D2 heavy chain control (SEQ ID NO:92) were selected based on structural analysis. The selected residues were randomized to find single amino acid substitution variants with improved HER2 ECD domain II binding. Antibodies with these single point mutations were paired with Anti-HER2_D4 light chain control (SEQ ID NO:87) and expressed in Expi293 cells and antibodies in cell culture supernatants were screened for recombinant HER2 ECD binding using SPR. Variants with improved HER2 ECD domain II were selected and expressed with SEQ ID NO:10 light chain in Expi293 cells and purified for additional SPR binding evaluation.

Affinity matured anti-HER2_D2 heavy chain sequences comprising the V_{H} region of SEQ ID NOS:1-2 and 60-70 were paired with anti-HER2_D4 light chain control (SEQ ID NO:87) for HER2 binding K_{D} measurement. Results are shown in Table 8.

TABLE 8

| | | HER2 binding |
|---|---|---|
| LC | HC | K_{D} (nM) |
| Anti-HER2_D4 light | Anti-HER2_D2 heavy chain | 42 |
| chain control | control (SEQ ID NO: 92) | |
| (SEQ ID NO: 87) | SEQ ID NO: 60 | 28 |
| | SEQ ID NO: 61 | 17.5 |
| | SEQ ID NO: 62 | 18.3 |
| | SEQ ID NO: 63 | 22.1 |
| | SEQ ID NO: 1 | 28 |
| | SEQ ID NO: 64 | 21 |
| | SEQ ID NO: 65 | 35 |
| | SEQ ID NO: 66 | 32.3 |
| | SEQ ID NO: 67 | 6.1 |
| | SEQ ID NO: 68 | 9.8 |
| | SEQ ID NO: 69 | 10.2 |
| | SEQ ID NO: 2 | 28 |
| | SEQ ID NO: 70 | 30 |

Affinity matured anti-HER2_D2 heavy chain sequences comprising the V_{H} region of SEQ ID NOS:1-2 and 60-70 paired with anti-HER2_D4 light chain control (SEQ ID NO:87) showed HER2 binding and all showed HER2 binding improvement compared to Anti-HER2_D2 heavy chain control.

Affinity matured anti-HER2 light chain sequence (SEQ ID NO:10) was paired with affinity matured anti-HER2_D2 heavy chain sequences comprising the V_{H} region of SEQ ID NOS:1-3 for HER2 binding K_{D} measurement. Results are shown in Table 9.

TABLE 9

| LC | HC | HER2 binding K_{D} (nM) |
|---|---|---|
| SEQ ID NO: 10 | SEQ ID NO: 1 | 4.2 |
| | SEQ ID NO: 2 | 6.2 |
| | SEQ ID NO: 3 | 2.1 |

SEQ ID NO:10 light chain paired with affinity matured anti-HER2_D2 heavy chain sequences comprising the V_{H} region of SEQ ID NOS:1-3 showed improved HER2 binding affinity (4.2, 6.2, 2.1 K_{D} respectively). As discussed above and shown in Table 7, Anti-HER2_D2 light chain control light chain paired with Anti-HER2_D2 heavy chain control had a HER2 binding affinity of 2.9 K_{D}. Therefore, SEQ ID NO:10 light chain paired with affinity matured anti-HER2_D2 heavy chain sequences comprising the V_{H} region of SEQ ID NOS:3 binds HER2 with a higher affinity than the control.

### Example 3. In Vitro ADCC/ADCP of Anti-HER2 Bispecific Antibodies

A human ADCC Reporter Bioassay, V variant kit (Promega G7018) was used to assess activation of human FcγRIIIa, while a human FcgRIIa ADCP Reporter Bioassay kit (Promega G9995) was used to measure activation of the human FcγRIIa reporter of the bispecific antibodies according to the combinations in Table 10. The kit contains all of the components described below. Several cell lines with varying expression levels of HER2 and TfR were tested. The cells SKBR3 (ATCC HTB-30), ZR-75-30 (ATCC CRL-1504), BT-474 (ATCC HTB-20), OE-19 (Sigma 96071721), CHO-KI+HumanTfR (ChemPartner CRO agreement) were cultured in RPMI (Liffe Technologies 61870-036) supplemented with 10% FBS (Hyclone Bovine serum SH30080.03) and 1% Penicillin-Streptomycin (Life Technologies 15140-122) to exponential phase, washed twice with PBS and resuspended at 1.0×10⁶ cells/mL in RPMI supplemented with 10% FBS and 1% Penicillin/Streptomycin. White 96-well high binding Nunc plates (ThermoFisher) were coated with 25 μL of media containing 50,000 cells/well.

Antibody titrations were prepared in RPMI with 4% low IgG serum and 25 μl per well was added to the plates to opsonize cells, then covered and incubated for 30 minutes at 37 °C, 5% CO₂. During antibody opsonization, 3.5 mL of medium was pre-warmed to 37 °C and the FcγR reporter cells were quickly defrosted in 37 °C water bath, without inverting, then added to pre-warmed medium in a 15 mL conical tube with gentle mixing. After 30-minute opsonization, FcγR reporter cell line was added to each plate at 25 μl per well and incubated for 6 hours (hFcγRIIIa and hFcγRIIa activation for SKBR3, ZR-75-30, BT-474) or 16 hours (hFcγRIIIa and hFcγRIIa for CHO-KI+huTfR) at 37 °C, 5% CO₂. After incubation, plates were allowed to acclimate to room temperature and 75 μL per well of Bio-Glo luciferase substrate suspension (Promega) was added and luminescence measured on a Perkin Elmer Envision reader. Results are shown in Table 11.

TABLE 10

| | | Hole chain | | | | | | | |
|---|---|---|---|---|---|---|---|---|---|
| | | hole | hole.E | hole.D | hole.DE | hole.PG | hole.PG.D | hole.PG.E | hole.PG.DE |
| Knob | cis-LALA.CH3C.35.23.4.knob | Fc1 | Fc2 | Fc3 | Fc4 | Fc5 | Fc6 | Fc7 | Fc8 |
| chain | cis-LALA.D.CH3C.35.23.4.knob | Fc17 | Fc18 | Fc19 | Fc20 | Fc21 | Fc22 | Fc23 | Fc24 |
| | cis-LALA.E.CH3C.35.23.4.knob | Fc25 | Fc26 | Fc27 | Fc28 | Fc29 | Fc30 | Fc31 | Fc32 |
| | cis-LALA.DE.CH3C.35.23.4.knob | Fc33 | Fc34 | Fc35 | Fc36 | Fc37 | Fc38 | Fc39 | Fc40 |
| | cis-LALAPG.CH3C.35.23.4.knob | Fc41 | Fc42 | Fc43 | Fc44 | Fc45 | Fc46 | Fc47 | Fc48 |
| | cis-LALAPG.D.CH3C.35.23.4.knob | Fc49 | Fc50 | Fc51 | Fc52 | Fc53 | Fc54 | Fc55 | Fc56 |
| | cis-LALAPG.E.CH3C.35.23.4.knob | Fc57 | Fc58 | Fc59 | Fc60 | Fc61 | Fc62 | Fc63 | Fc64 |
| | cis-LALAPG.DE.CH3C.35.23.4.knob | Fc65 | Fc66 | Fc67 | Fc68 | Fc69 | Fc70 | Fc71 | Fc72 |

TABLE 11

| | Assay done | | | | | | |
|---|---|---|---|---|---|---|---|
| | TfR-mediated | | | | | | |
| | ADCC | HER2-mediated ADCC | | | ADCP | | |
| Cell line used | CHO-hTfR | BT-474 | OE19 | ZR-75-30 | OE19 | ZR-75-30 | SKBR3 |
| Anti-HER2_D4 (control) | 3.0E+04 | 4.3E+05 | 1.4E+06 | 4.8E+05 | 1.1E+05 | 3.6E+04 | 3.5E+04 |
| Fc1 | 2.8E+04 | 1.5E+04 | 4.9E+05 | 3.5E+04 | 9.2E+05 | 1.6E+05 | 3.1E+05 |
| Fc41 | 2.5E+04 | 3.9E+04 | 2.1E+05 | 2.8E+04 | 2.2E+05 | 8.9E+04 | 5.9E+04 |
| Fc5 | 2.7E+04 | 3.3E+04 | 2.2E+05 | 3.0E+04 | | 4.1E+04 | 3.8E+04 |
| Fc45 | 2.6E+04 | 2.3E+04 | 7.1E+04 | 2.4E+04 | 1.5E+05 | 3.6E+04 | 3.3E+04 |
| Fc42 | 2.7E+04 | 1.5E+05 | 6.5E+05 | 7.7E+04 | 2.6E+05 | 1.0E+05 | 5.7E+04 |
| Fc52 | 2.5E+04 | 4.6E+05 | 1.2E+06 | 2.1E+05 | 2.3E+05 | 8.5E+04 | 5.8E+04 |
| Fc44 | 2.6E+04 | 2.0E+05 | 7.3E+05 | 9.3E+04 | 1.6E+05 | 4.1E+04 | 3.9E+04 |
| Fc50 | 2.9E+04 | 3.2E+05 | 2.6E+06 | 1.9E+05 | 1.6E+06 | 3.4E+05 | 2.5E+05 |
| Fc68 | 2.9E+04 | 3.6E+05 | 1.8E+06 | 2.2E+05 | | | |
| Fc7 | 2.7E+04 | 3.1E+04 | 3.0E+05 | 5.0E+04 | | | |
| Fc24 | 1.4E+05 | 2.8E+05 | 1.6E+06 | 1.6E+05 | | | |
| Fc8 | 2.7E+04 | 8.9E+04 | 8.2E+05 | 1.6E+05 | | | |
| Fc40 | 2.5E+05 | 3.4E+05 | 2.0E+06 | 2.3E+05 | | | |
| Fc23 | 7.2E+04 | 1.3E+05 | 1.1E+06 | 7.1E+04 | | | |
| Fc25 | 4.8E+04 | | | 2.2E+05 | | | |
| Fc2 | 2.3E+04 | | | 2.2E+05 | | 1.6E+05 | 3.0E+05 |
| Fc26 | 4.7E+04 | | | 3.4E+05 | | | |

The aim was to develop Fc variants that did not increase TfR-mediated ADCC compared to the control and/or Fc1 and which also had a comparable level of HER2-mediated ADCC as the control and/or improved level of HER2-mediated ADCC levels compared to Fc1. As shown above in Table 11, Fc1, Fc41, Fc5, Fc45, Fc42, Fc52, Fc44, Fc50, Fc68, Fc7, Fc8, Fc2, Fc34, and Fc4 all had a comparable levels of TfR-mediated ADCC in TfR-overexpressing CHO cells as the control. Fc50 and Fc52 showed the highest level of HER2-mediated ADCC across all the tested HER2-overexpressing cell lines without increasing TfR-mediated ADCC activation.

ADCP levels of Fc1, Fc41, Fc5, Fc45, Fc42, Fc52, Fc44, and Fc50 variants are also shown in Table 11 compared to the control across HER2-over expressing cell lines, i.e., OE19, ZR-75-30, and SKBR3.

### Example 4. In Vitro Growth Inhibition of Anti-HER2 Bispecific Antibodies

A growth inhibition assay was used to determine the viability of cells after treatment with different antibodies for different durations. Several cell lines with varying expression levels of HER2 and TfR were tested. The cells SKBR3 (ATCC HTB-30), ZR-75-30 (ATCC CRL-1504), BT-474 (ATCC HTB-20), OE-19 (Sigma 96071721), CHO-KI+HumanTfR (ChemPartner CRO agreement) were cultured in RPMI (Life Technologies 61870-036) supplemented with 10% FBS (Hyclone Bovine serum SH30080.03) and 1% Penicillin-Streptomycin (Life Technologies 15140-122) to exponential phase. After washing with PBS, the cells were resuspended at 1.0×10⁵ cells/mL in RPMI supplemented with 10% FBS and 1% Penicillin/Streptomycin. Black Poly-D-Lysine plates (Corning 354640) were coated with 100 μl of cell culture media containing 10,000 cells/well. The plates were incubated for 24 hrs in a 37 °C, 5% CO₂ incubator.

Antibody titrations were prepared in RPMI with 10% FBS serum and 1% Penicillin/Streptomycin. The antibodies were added to each plate at 65 μl per well, then covered and incubated for 72 hrs (for OE-19 cell line only) and at 37 °C, 5% CO₂. For BT-474 and ZR-75-30 cell lines, an additional 65 μl of antibody were added after 72 hrs and then incubated for another 72 hrs at 37 °C, 5% CO₂.

On Day 7, cell growth was determined using 5 μL of WST-1 reagent (Sigma Aldrich) in 50 μL of growth media. The plate was incubated for 4 hours in the presence of WST-1 reagent, and absorbance was determined at 440 nm. The percent of growth inhibition/proliferation was calculated based on A440 nM and was normalized to the untreated control.

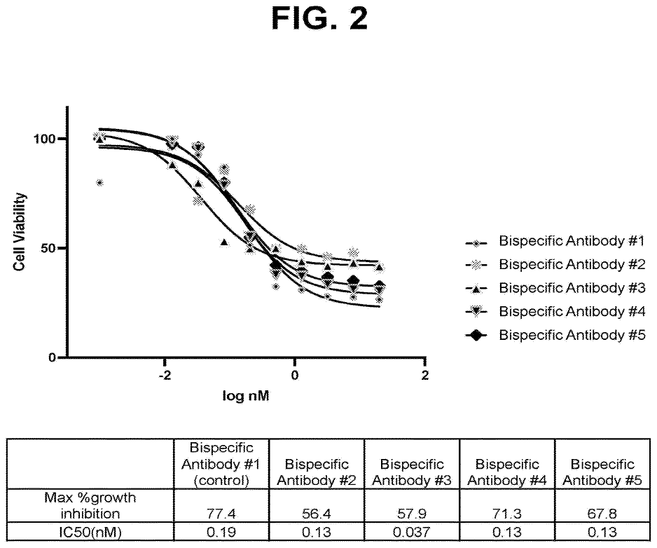

Growth inhibition assay results on ZR-75-30 cells for the different antibodies in Table 12 as well as IC50 and max % growth inhibition values are shown in FIG. 2. Each of Bispecific Antibody #2, #3, #4, and #5 showed improved EC50 value/efficacy compared to the control. Bispecific Antibody #4 and 5 showed comparable max % growth inhibition/efficacy to the control.

TABLE 12

| | HC1 | HC2 | LC |
|---|---|---|---|
| Bispecific | SEQ ID NO: 20 | SEQ ID NO: 24 | SEQ ID NO: 19 |
| Antibody #1 | | | |
| (control) | | | |
| Bispecific | SEQ ID NO: 93 | SEQ ID NO: 92 | SEQ ID NO: 10 |
| Antibody #2 | | | |
| Bispecific | SEQ ID NO: 37 | SEQ ID NO: 26 | SEQ ID NO: 10 |
| Antibody #3 | | | |
| Bispecific | SEQ ID NO: 37 | SEQ ID NO: 27 | SEQ ID NO: 10 |
| Antibody #4 | | | |
| Bispecific | SEQ ID NO: 37 | SEQ ID NO: 25 | SEQ ID NO: 10 |
| Antibody #5 | | | |

### Example 5. In Vivo Xenograft Studies with ATV:CLC Bispecific Antibody

Two human HER2+ cell lines were used to evaluate response of ATV:CLC bispecific antibody #1 in subcutaneous xenograft models in immunodeficient (NOD/SCID) mice. All molecules were prepared in the same formulation buffer (10 mM NaAcetate, 6% sucrose, pH5.5) or PBS/saline except for trastuzumab (Clinical Herceptin) and pertuzumab (Clinical Perjeta) which were purchased and prepared according to instructions and/or diluted further with PBS or saline.

TABLE 13
Anti-HER2 bispecific molecules

| | VH_D4 | VH_D2 | | |
|---|---|---|---|---|
| Patent nomenclature | (knob side) | (hole side) | LC | Fc Mods |
| Common light chain | SEQ ID NO: 108 | SEQ ID NO: 109 | SEQ ID NO: 19 | No TV |
| (CLC) bispecific | | | | |
| antibody control | | | | |
| (Herceptarg) | | | | |
| Common light chain | SEQ ID NO: 15 | SEQ ID NO: 3 | SEQ ID NO: 10 | No TV |
| (CLC) bispecific | | | | |
| antibody control #2 | | | | |
| ATV:Common light | SEQ ID NO: 108 | SEQ ID NO: 109 | SEQ ID NO: 19 | Knob side: |
| chain (CLC) bispecific | | | | TV35.23.4, cis- |
| antibody #1 | | | | LALA |
| ATV:Common light | SEQ ID NO: 15 | SEQ ID NO: 3 | SEQ ID NO: 10 | Knob side: |
| chain (CLC) bispecific | | | | TV35.23.4, cis- |
| antibody #2 | | | | LALA |
| ATV:Common light | SEQ ID NO: 15 | SEQ ID NO: 3 | SEQ ID NO: 10 | Knob side: |
| chain (CLC) bispecific | | | | TV35.23.4, cis- |
| antibody #3 | | | | LALAPS, S239D |
| | | | | Hole side: I332E |
| ATV:Common light | SEQ ID NO: 15 | SEQ ID NO: 3 | SEQ ID NO: 10 | Knob side: |
| chain (CLC) bispecific | | | | TV35.23.4, cis- |
| antibody #7 | | | | LALAPS, S239D |
| | | | | and I332E |
| | | | | Hole side: S239D |
| | | | | and I332E |

For the BT-474 breast cancer cell line-derived xenograft (CDX) model, female NSG (NOD scid gamma) mice (6-7 weeks of age) were injected subcutaneously into the axilla with BT-474 cells and treatment initiated six days following inoculation when tumors were between 100-200 mm³ in volume. Mice were randomized into treatment groups based on mean tumor volume, with n=11 mice per group. Trastuzumab and pertuzumab combination treatment at 40+40 mg/kg or ATV:CLC bispecific antibody #1 at 80 mg/kg was administered via intraperitoneal (IP) injection. Tumor volumes were measured three times per week using calipers.

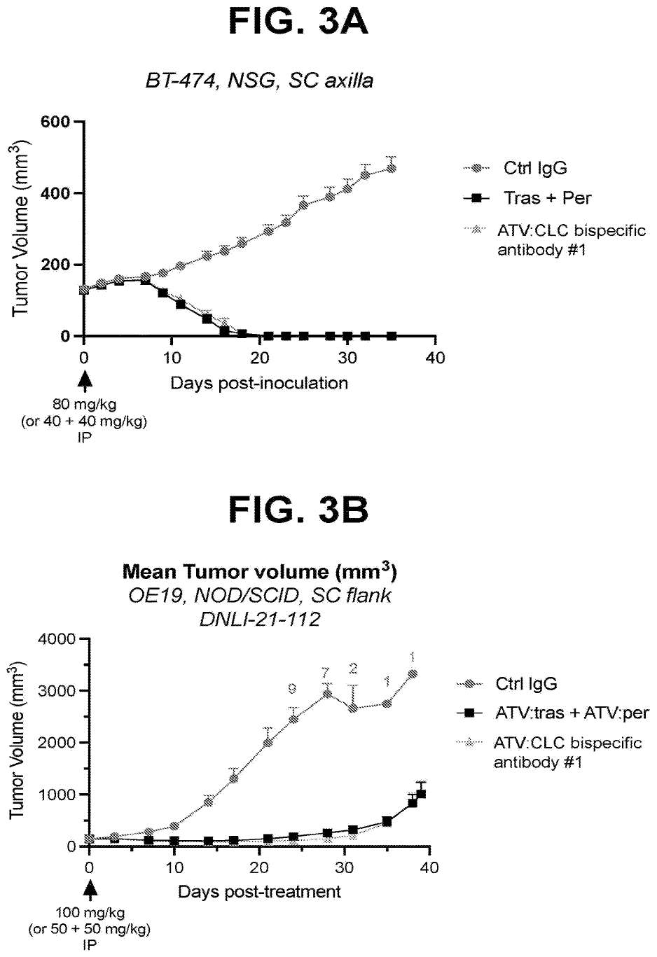

In a relatively trastuzumab-sensitive BT-474 xenograft model, ATV:CLC bispecific antibody #1 showed equivalent inhibition of tumor growth following a single dose compared with trastuzumab and pertuzumab, with complete regression of the tumors for the entire treatment group after 21 days (FIG. 3A).

For the OE19 gastro-esophageal junction CDX model, female NOD/SCID mice (6-8 weeks of age) were injected subcutaneously with OE19 cells in the right upper flank region and treatment initiated one week following inoculation when tumors were between 100-200 mm³ in volume. Mice were randomized into treatment groups based on a matched distribution/stratified method, with n=11 mice per group. ATV:trastuzumab and ATV:pertuzumab combination treatment at 50+50 mg/kg or ATV:CLC bispecific antibody #1 at 100 mg/kg was administered via IP injection. Tumor volumes were measured three times per week using calipers.

In the OE19 xenograft model, which shows relative resistance to trastuzumab and pertuzumab combination treatment, ATV:CLC bispecific antibody #1 and the combination of ATVcis-LALA:trastuzumab and ATVcis-LALA:pertuzumab both showed significant tumor growth delay compared to the control group (FIG. 3B). All groups start with n=11 mice per group. Numbers on graph represent the number of animals remaining in control group after subset of animals reached humane endpoint. One animal found dead in ATV combo group on day 17 without apparent cause. ATV:trastuzumab and ATV:pertuzumab are traztuzumab and pertuzumab antibodies comprising an Fc modification that binds to TfR (“TV”) and cis-LALA mutations. These in vivo results are in line with in vitro growth inhibition data which demonstrated an increased potency and increased maximal effect of ATV:HER2 (anti-HER2 molecules with TV) in the OE19 cell line compared to anti-HER2 molecules lacking the TfR binding Fc modification.

In a follow-up lower dose study, to study the effect of TfR binding, ATV:CLC bispecific antibody #1 was compared with a CLC bispecific antibody control which had identical Fabs as ATV:CLC bispecific antibody #1 but lacked the TfR binding Fc modification. For the BT-474 breast cancer CDX model, female NOD/SCID mice (6-8 weeks of age) were implanted with estrogen pellets (0.36 mg, 17B-estradiol, 60 day pellet) one day before tumor inoculation. BT-474 cells were then injected subcutaneously into the mammary fat pad and treatment initiated eight days following inoculation when tumors were between 100-200 mm³ in volume. Mice were randomized into treatment groups based on a matched distribution/stratified method, with n=11 mice per group. Tumor volumes were measured two times per week using calipers.

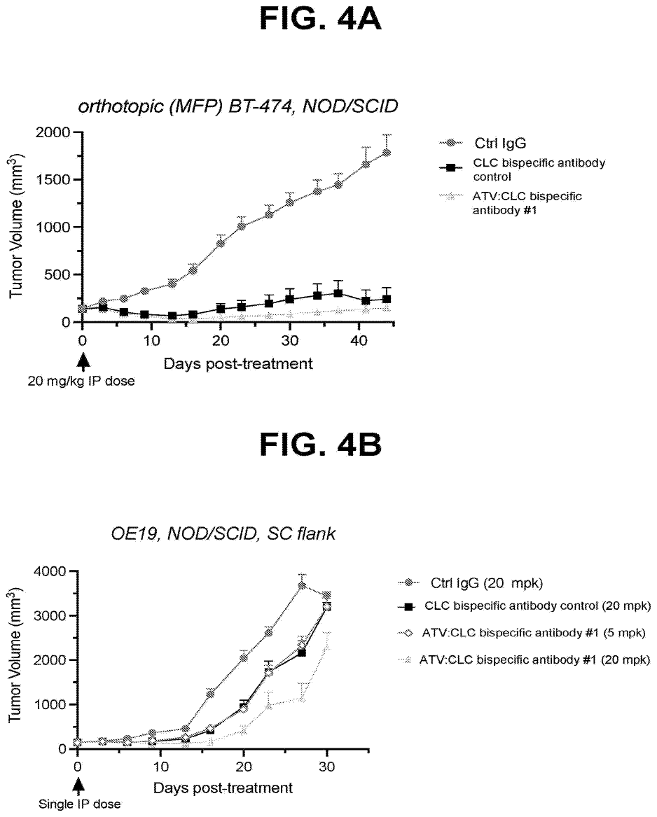

A single 20 mg/kg dose of ATV:CLC bispecific antibody #1 administered intraperitoneally showed similar tumor growth delay as the CLC bispecific antibody control (no TfR binding) in the sensitive BT-474 xenograft model (FIG. 4A). ATV:CLC bispecific antibody #1 showed an improved response in the more resistant OE19 xenograft model at equal doses of 20 mg/kg and equivalent anti-tumor response at a four-fold lower dose (5 mg/kg) than the CLC bispecific antibody control (FIG. 4B).

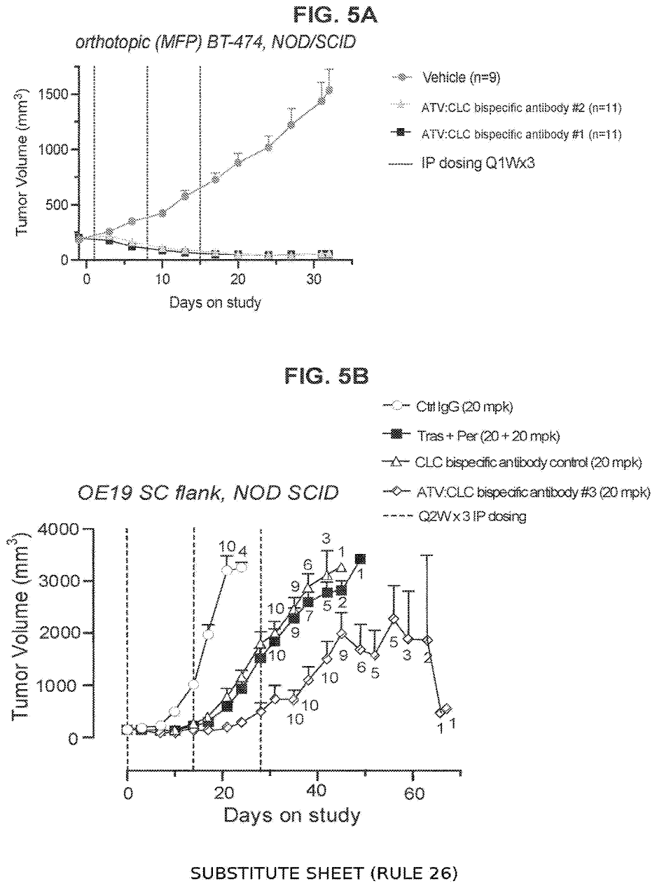

In a further bridging study, ATV:CLC bispecific antibody #1 and #2 were compared in multidose xenograft studies. In the BT-474 model, mice (n=11 for each group) were dosed Q1W via IP, i.e., mice were administered a single dose per week for 3 weeks. ATV:CLC bispecific antibody #1 and ATV:CLC bispecific antibody #2 showed equal inhibition and delay of tumor growth (FIG. 5A). Additionally, the same groups were dosed in combination with daily 50 mg/kg oral dosing of tucatinib for 21 days but no additional improvements were seen with ATV:CLC bispecific antibody #1 or #2.

Lastly, an Fc engineered variant of ATV:CLC bispecific antibody #2, ATV:CLC bispecific antibody #3 (comprising additional Fc modifications, e.g., P329S, I332E, and S239D) was also compared to anti-HER2 molecules lacking TfR binding in a multidose OE19 xenograft study. Mice were dosed Q2W via IP, i.e., mice were administered a single dose every 2 weeks for 6 weeks. ATV:CLC bispecific antibody #3 showed increased tumor growth delay and increased survival compared to the combination of trastuzumab and pertuzumab or the CLC bispecific antibody control (FIG. 5B). All groups start with n=11 mice per group. Numbers on graph represent the number of animals remaining in group after subset of animals reached humane endpoint.

### Example 6. Brain Uptake and Distribution of ATV:CLC Bispecific Antibody

TfR^{mu/hu} KI mice (see, e.g., International Publication No. WO 2018/152285) were administered a single 25 mg/kg IV dose of CLC bispecific antibody control or ATV:CLC bispecific antibody #3 (n=4/group). In-life blood was collected at 30 minutes and 6 hours and terminal blood and fresh frozen brain were collected at 1, 4, 7, and 10 days post-dose to evaluate huIgG concentrations in plasma and brain lysate via ELISA.

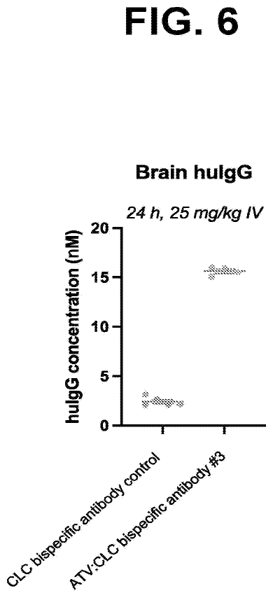

Plasma and brain concentrations were measured following a single dose of bispecific CLC bispecific antibody control or ATV:CLC bispecific antibody #3 in TfR^{mu/hu} KI mice. Brain concentrations of ATV:CLC bispecific antibody #3 were approximately 6.5-fold higher at 24 h post-dose compared to the CLC bispecific antibody control (FIG. 6). This demonstrates TfR-mediated brain delivery for the ATV molecules. Similarly, in a time course study, ATV:CLC bispecific antibody #2, #3, and #7 showed approximately 4-5-fold higher brain concentrations 24 h and up to ~2-fold higher brain concentrations 4 days after IV dosing.

Furthermore, immunohistochemistry of dosed molecules in the brain was performed. One fresh brain hemisphere per animal was immersion fixed for approximately 24 h at 4 C for immunohistochemistry before cryoprotecting in sucrose and sectioning on a freezing microtome. Coronal brain sections (40 μm) were selected for each animal and stained by incubating in blocking buffer (1% BSA+1× fish gelatin+0.5% Triton X-100+0.01% sodium azide in PBS) for three hours at room temperature. Sections were then incubated overnight in dilution buffer (1% BSA+0.3% Triton X-100+0.01% sodium azide in PBS) containing primary/secondary antibodies (NeuN, Abcam, ab177487 and donkey-anti-huIgG, Jackson, 709-606-149) at 4 C, washed three times for 15 minutes each in PBS with 0.3% Triton X-100, and incubated for three hours in dilution buffer containing secondary antibodies (donkey-anti-rabbit, Invitrogen, A21206) and DAPI (5 μg/mL, Invitrogen, D1306), and washed three times for 15 minutes each in PBS with 0.3% Triton X-100 before mounting and coverslipping with Prolong Glass (Invitrogen, P36984). Slides were imaged using a Leica SP8 confocal microscope at 20× magnification and segmentation and visualization performed using Imaris.

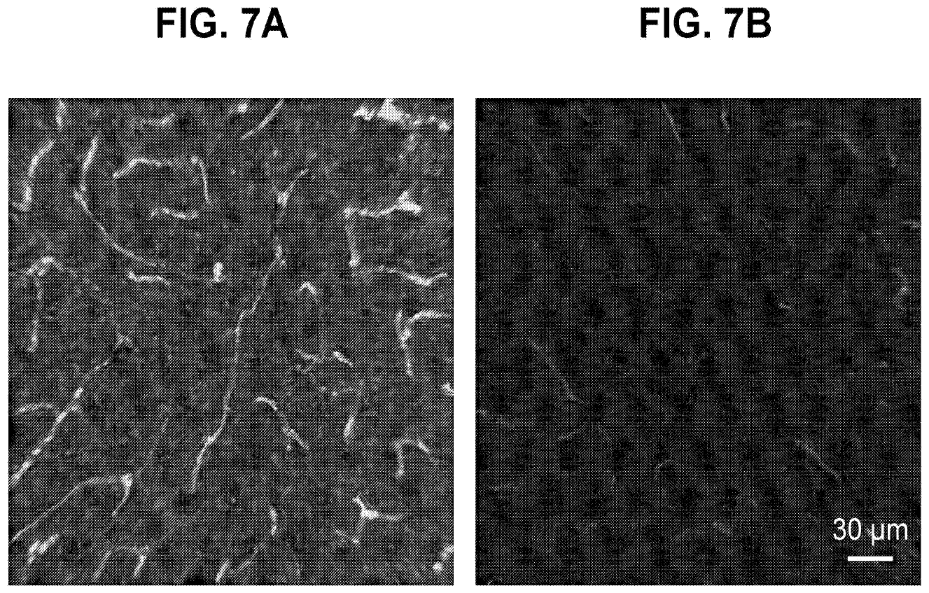

Immunohistochemistry for the huIgG backbone of the dosed molecules revealed a broad distribution of ATV:CLC bispecific antibody #3 across the normal brain, localizing within blood vessels and NeuN+ neurons along with diffuse signal within the parenchyma (FIG. 7A). In contrast, the CLC bispecific antibody control showed limited entry or distribution within the brain tissue (FIG. 7B). This is in line with the significantly lower brain concentrations observed for non-TV anti-HER2 molecules, i.e., molecules without TfR binding. Similar results, i.e., vascular and neuronal/parenchymal localization, were observed with ATV:CLC bispecific antibody #2 and ATV:CLC bispecific antibody #7.

### Example 7. Plasma PK of ATV:CLC Bispecific Antibodies in Cynomolgus Monkeys

To assess the impact of the Fc modifications on systemic clearance, Fc modification variants were compared. Since TV35.23.4 (i.e., CH3C.35.23.4) does not have cyno cross-reactivity, i.e., does not bind cyno TfR, bispecific HER2 ATVs with TV35.21 (see, CH3C.35.21 in Table A above) instead of TV35.23.4 were used. As shown in Table 14 below, these molecules use the same Fabs as ATV:CLC bispecific antibody #2, #3, and #7 used in the mouse studies above.

ATV:CLC bispecific antibody #4, #5, and #6 were compared to Clinical Herceptin (trastuzumab) and serum concentrations of huIgG were measured at various time points following a single 50 mg/kg intravenous dose in female cynomolgus monkeys (n=3/group).

TABLE 14
Anti-HER2 bispecific molecules for cyno study

| | VH_D4 | VH D2 | | |
|---|---|---|---|---|
| | (knob side) | (hole side) | LC | Fc Modifications |
| ATV:CLC bispecific | SEQ ID NO: 15 | SEQ ID NO: 3 | SEQ ID NO: 10 | Knob side: TV35.21, |
| antibody #4 | | | | cis-LALA |
| ATV:CLC bispecific | SEQ ID NO: 15 | SEQ ID NO: 3 | SEQ ID NO: 10 | Knob side: TV35.21, cis- |
| antibody #5 | | | | LALAPS, S239D |
| | | | | Hole side: I332E |
| ATV:CLC bispecific | SEQ ID NO: 15 | SEQ ID NO: 3 | SEQ ID NO: 10 | Knob side: TV35.21, cis- |
| antibody #6 | | | | LALAPS, S239D and I332E |
| | | | | Hole side: S239D and I332E |

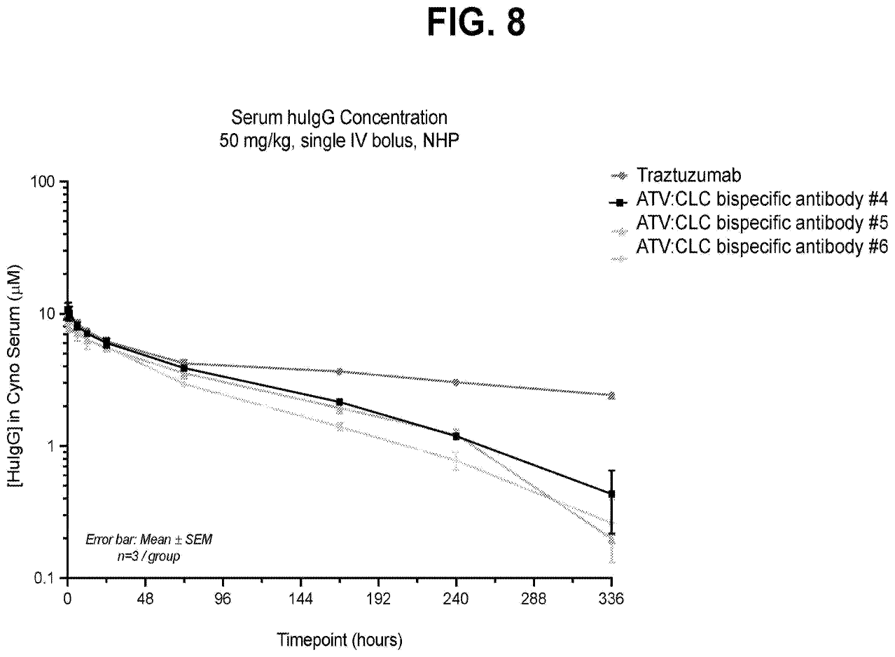

All ATV:HER2 molecules showed a more rapid clearance from systemic circulation compared to Herceptin (trastuzumab), as expected due to TfR-mediated clearance (FIG. 8).

### Example 8. In Vitro ADCC/ADCP of Anti-HER2 Bispecific Antibodies in NK Cells

A cell-based antibody-dependent cell cytotoxicity (ADCC) assay was used to assess whether the differences in Fc gamma receptor binding affinities for the different Fc mutants impacted HER2-mediated tumor cell or TfR-mediated cell killing using isolated human NK cells.

NK cells were isolated from whole blood and were used to assess activation of human FcγRIIIa. Blood was collected on Trizma. The cells were isolated according to the RosetteSep Human NK cell enrichment Protocol (Stemcell 15065). RosetteSep Cocktail was added to Blood Samples in a SepMate tube and left at RT for 15 mins. After incubation the samples were diluted with an equal volume of PBS (Gibco 10010-0310) and 10% FBS (Hyclone Bovine serum SH30080.03). The diluted sample is then added to the Density gradient media Lymphoprep (Stemcell 07801) and centrifuged for 10 mins. The enriched cells are then collected and wash 2 times with PBS. Finally, 20 ng/ml of IL-21 is added to the cells and then left overnight for next day use.

Cell lines with varying expression levels of HER2 and TfR were tested. The cells SKBR3 (ATCC HTB-30) and CHO-KI+HumanTfR (ChemPartner CRO agreement) were cultured in RPMI (Life Technologies 61870-036) supplemented with 10% FBS (Hyclone Bovine serum SH30080.03) and 1% Penicillin-Streptomycin (Life Technologies 15140-122) to exponential phase, washed twice with PBS and resuspended at 1.0×10⁶ cells/mL in RPMI supplemented with 10% FBS and 1% Penicillin/Streptomycin.

Clear 96-well non-treated V bottom plates (Costar 3897) were coated with 25 μL of media containing 50,000 cells/well. Antibody titrations were prepared in RPMI with 10% FBS serum and 25 μl per well was added to the plates to opsonize cells, then covered and incubated for 30 minutes at 37 °C, 5% CO₂. During antibody opsonization, the NK cells were washed one time with media containing RPMI and 10% FBS. Cells were counted and an ET ratio of 25:1 was used for cell density. After 30-minute opsonization, NK cells were added to each plate at 25 μl per well and incubated for 4 hours. After incubation, plates were allowed to acclimate to room temperature and spun down at 300×g for 5 mins. 50 μL of the supernatant was removed to a White 96-well clear bottom plate (Thermo 165306). 50 μl of CytoTox 96® Assay Reagent was added to each well of the plate containing the supernatant. The plate was covered to protect it from light and incubate for 30 minutes at room temperature. Stop Solution is added, and the absorbance signal is measured at 490 nm in a plate reader. Released LDH in culture supernatants is measured with a 30-minute coupled enzymatic assay, which results in the conversion of a tetrazolium salt (iodonitrotetrazolium violet; INT) into a red formazan product. The amount of color formed is proportional to the number of lysed cells.

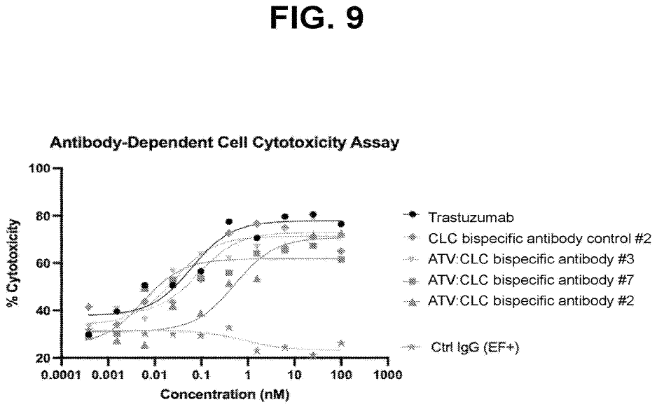

Interestingly, in this assay on HER2 expressing tumor cells, the cis-LALA modification resulted in only a slight rightward shift in the curve indicating a slight decrease in potency, but an equal maximum effect of cell killing was observed as the non-TV anti-HER2 bispecific, i.e., CLC bispecific antibody control #2 and trastuzumab (FIG. 9).

No cell killing was observed for TfR-expressing (HER2-) cells with cis-LALA or additional Fc mutations, suggesting these molecules would not negatively impact TfR-expressing cells.

The ADCP reporter assay to measure FcgRIIA activation also demonstrated that ATV:CLC bispecific #2 and Fc variants (ATV:CLC bispecific #3 and #7) showed similar receptor activation as each other which was greater than trastuzumab and slightly less than CLC bispecific antibody control #2.

### Example 9. FcgR Binding Assay of ATV:CLC Bispecific Antibodies

Fc gamma receptor binding affinities of engineered anti-HER2 antibodies were measured by SPR using a Biacore 8K instrument. Biotinylated recombinant Fc gamma receptors were captured on Biacore™ Series SA sensor chips followed by injections of serial 3-fold dilutions of Fc-engineered anti-Her2 antibodies at a flow rate of 30 L/min. Each sample was analyzed using five 60 second injections with increasing antibody concentrations followed by a 5 minute dissociation. A 1:1 Languir model of simultaneous fitting of k on and k off was used for kinetics analysis.

TABLE 15
Fc gamma receptor binding affinities of
Fc-engineered anti-Her2 antibodies

| | | ATV:CLC | ATV:CLC | ATV:CLC |
|---|---|---|---|---|
| | | bispecific | bispecific | bispecific |
| | Trastuzumab | antibody #4 | antibody #5 | antibody #6 |
| FcgR2a | 48 nM | 430 nM | 39 nM | 13 nM |
| FcgR3a(V158) | 890 nM | 3.2 μM | 560 nM | 760 nM |

Increased FcgR affinities are observed in the Fc variants engineered with S239D and I332E, i.e., ATV:CLC bispecific antibody #5 and #6, compared with ATV:CLC bispecific antibody #4 and trastuzumab. However, taken together with the results in the ADCC assays described above, this increased affinity may only apply when the antibodies are bound to the Fab target, i.e., HER2.

### IX. Exemplary Embodiments

Exemplary embodiments provided in accordance with the presently disclosed subject matter include, but are not limited to, the claims and the following embodiments:
1. An isolated antibody comprising one or more complementarity determining regions (CDRs) selected from the group consisting of:
   (a) a heavy chain CDR1 comprising the amino acid sequence of SEQ ID NO:89;
   (b) a heavy chain CDR2 comprising the amino acid sequence of SEQ ID NO:90; and
   (c) a heavy chain CDR3 comprising the amino acid sequence of SEQ ID NO:91,
   wherein at least one of:
      X₁ in SEQ ID NO: 89 is not T;
      X₂ in SEQ ID NO: 89 is not F;
      X₃ in SEQ ID NO: 89 is not T;
      X₁ in SEQ ID NO: 90 is not N;
      X₂ in SEQ ID NO: 90 is not N;
      X₃ in SEQ ID NO: 90 is not S;
      X₄ in SEQ ID NO: 90 is not G;
      X₅ in SEQ ID NO: 90 is not G;
      X₆ in SEQ ID NO: 90 is not Q;
      X₁ in SEQ ID NO: 91 is not L;
      X₂ in SEQ ID NO: 91 is not G;
      X₃ in SEQ ID NO: 91 is not P; and
      X₄ in SEQ ID NO: 91 is not S.
2. The isolated antibody of embodiment 1, wherein the heavy chain CDR1 comprises the amino acid sequence of SEQ ID NO:89, wherein X₁ is N, K, M, or H.
3. The isolated antibody of embodiment 1, wherein the heavy chain CDR2 comprises the amino acid sequence of SEQ ID NO:90, wherein X₅ is Q.
4. The isolated antibody of embodiment 1, wherein the heavy chain CDR2 comprises the amino acid sequence of SEQ ID NO:90, wherein X₆ is R, H, or T.
5. The isolated antibody of embodiment 1, wherein the heavy chain CDR3 comprises the amino acid sequence of SEQ ID NO:91, wherein X₄ is W, F, D, L, or Y.
6. The isolated antibody of embodiment 1, wherein the heavy chain CDR3 comprises the amino acid sequence of SEQ ID NO:91, wherein X₄ is L.
7. The isolated antibody of embodiment 1, comprising one or more CDRs selected from the group consisting of:
   (a) a heavy chain CDR1 comprising the amino acid sequence of SEQ ID NO:89;
   (b) a heavy chain CDR2 comprising the amino acid sequence of SEQ ID NO:90, wherein X₅ is Q; and
   (c) a heavy chain CDR3 comprising the amino acid sequence of SEQ ID NO:91, wherein X₄ is L.
8. The isolated antibody of embodiment 1, comprising one or more CDRs selected from the group consisting of:
   (a) a heavy chain CDR1 having at least 90% sequence identity to an amino acid sequence selected from the group consisting of SEQ ID NOS:4 and 49-52 or having up to two amino acid substitutions relative to an amino acid sequence selected from the group consisting of SEQ ID NOS:4 and 49-52;
   (b) a heavy chain CDR2 having at least 90% sequence identity to an amino acid sequence selected from the group consisting of SEQ ID NOS:5-6 and 53-55 or having up to two amino acid substitutions relative to an amino acid sequence selected from the group consisting of SEQ ID NOS:5-6 and 53-55; and
   (c) a heavy chain CDR3 having at least 90% sequence identity to an amino acid sequence selected from the group consisting of SEQ ID NOS:7-8 and 56-59 or having up to two amino acid substitutions relative to an amino acid sequence selected from the group consisting of SEQ ID NOS:7-8 and 56-59.
9. The isolated antibody of embodiment 8, comprising one or more CDRs selected from the group consisting of:
   (a) a heavy chain CDR1 having at least 90% sequence identity to the amino acid sequence of SEQ ID NO:4 or having up to two amino acid substitutions relative to the amino acid sequence of SEQ ID NO:4;
   (b) a heavy chain CDR2 having at least 90% sequence identity to the amino acid sequence of SEQ ID NO:5 or SEQ ID NO:6 or having up to two amino acid substitutions relative to the amino acid sequence of SEQ ID NO:5 or SEQ ID NO:6; and
   (c) a heavy chain CDR3 having at least 90% sequence identity to the amino acid sequence of SEQ ID NO:7 or SEQ ID NO:8 or having up to two amino acid substitutions relative to the amino acid sequence of SEQ ID NO:7 or SEQ ID NO:8.
10. The isolated antibody of embodiment 9, comprising one or more CDRs selected from the group consisting of:
   (a) a heavy chain CDR1 comprising the amino acid sequence of SEQ ID NO:4;
   (b) a heavy chain CDR2 comprising the amino acid sequence of SEQ ID NO:5 or SEQ ID NO:6; and
   (c) a heavy chain CDR3 comprising the amino acid sequence of SEQ ID NO:7 or SEQ ID NO:8.
11. The isolated antibody of embodiment 10, comprising one or more CDRs selected from the group consisting of:
   (a) a heavy chain CDR1 comprising the amino acid sequence of SEQ ID NO:4;
   (b) a heavy chain CDR2 comprising the amino acid sequence of SEQ ID NO:6; and
   (c) a heavy chain CDR3 comprising the amino acid sequence of SEQ ID NO:7.
12. The isolated antibody of embodiment 10, comprising one or more CDRs selected from the group consisting of:
   (a) a heavy chain CDR1 comprising the amino acid sequence of SEQ ID NO:4;
   (b) a heavy chain CDR2 comprising the amino acid sequence of SEQ ID NO:5; and
   (c) a heavy chain CDR3 comprising the amino acid sequence of SEQ ID NO:8.
13. The isolated antibody of embodiment 10, comprising one or more CDRs selected from the group consisting of:
   (a) a heavy chain CDR1 comprising the amino acid sequence of SEQ ID NO:4;
   (b) a heavy chain CDR2 comprising the amino acid sequence of SEQ ID NO:6; and
   (c) a heavy chain CDR3 comprising the amino acid sequence of SEQ ID NO:8.
14. The isolated antibody of embodiment 8, comprising a heavy chain variable region comprising an amino acid sequence having at least 90% sequence identity to any one of SEQ ID NOS:1-3.
15. The isolated antibody of embodiment 8, comprising a heavy chain variable region comprising the amino acid sequence of any one of SEQ ID NOS:1-3.
16. An isolated antibody comprising:
   (a) a light chain CDR3 comprising the amino acid sequence of SEQ ID NO:13 or 14.
17. The isolated antibody of embodiment 16, further comprising one or more CDRs selected from the group consisting of:
   (b) a light chain CDR1 having at least 90% sequence identity to the amino acid sequence of SEQ ID NO:11 or having up to two amino acid substitutions relative to the amino acid sequence of SEQ ID NO:11; and
   (c) a light chain CDR2 having up to two amino acid substitutions relative to the amino acid sequence of SEQ ID NO:12.
18. The isolated antibody of embodiment 16, further comprising one or more CDRs selected from the group consisting of:
   (b) a light chain CDR1 comprising the amino acid sequence of SEQ ID NO:11; and
   (c) a light chain CDR2 comprising the amino acid sequence of SEQ ID NO:12.
19. The isolated antibody of any one of embodiments 16 to 18, wherein the light chain CDR3 comprises the amino acid sequence of SEQ ID NO:13.
20. The isolated antibody of any one of embodiments 16 to 18, wherein the light chain CDR3 comprises the amino acid sequence of SEQ ID NO:14.
21. The isolated antibody of embodiment 17, comprising a light chain variable region comprising an amino acid sequence having at least 90% sequence identity to any one of SEQ ID NOS:9-10.
22. The isolated antibody of embodiment 17, comprising a light chain variable region comprising the amino acid sequence of any one of SEQ ID NOS:9-10.
23. An isolated antibody comprising an antigen binding site comprising:
   (a) a heavy chain CDR1 having at least 90% sequence identity to an amino acid sequence selected from the group consisting of SEQ ID NOS:4 and 49-52 or having up to two amino acid substitutions relative to an amino acid sequence selected from the group consisting of SEQ ID NOS:4 and 49-52;
   (b) a heavy chain CDR2 having at least 90% sequence identity to an amino acid sequence selected from the group consisting of SEQ ID NOS:5-6 and 53-55 or having up to two amino acid substitutions relative to an amino acid sequence selected from the group consisting of SEQ ID NOS:5-6 and 53-55; and
   (c) a heavy chain CDR3 having at least 90% sequence identity to an amino acid sequence selected from the group consisting of SEQ ID NOS:7-8 and 56-59 or having up to two amino acid substitutions relative to an amino acid sequence selected from the group consisting of SEQ ID NOS:7-8 and 56-59;
   (d) a light chain CDR1 having at least 90% sequence identity to the amino acid sequence of SEQ ID NO:11 or having up to two amino acid substitutions relative to the amino acid sequence of SEQ ID NO:11;
   (e) a light chain CDR2 having up to two amino acid substitutions relative to the amino acid sequence of SEQ ID NO:12; and
   (f) a light chain CDR3 comprising the amino acid sequence of SEQ ID NO:13 or 14.
24. The isolated antibody of embodiment 23, wherein the antigen binding site comprises:
   (a) a heavy chain CDR1 having at least 90% sequence identity to the amino acid sequence of SEQ ID NO:4 or having up to two amino acid substitutions relative to the amino acid sequence of SEQ ID NO:4;
   (b) a heavy chain CDR2 having at least 90% sequence identity to the amino acid sequence of SEQ ID NO:5 or SEQ ID NO:6 or having up to two amino acid substitutions relative to the amino acid sequence of SEQ ID NO:5 or SEQ ID NO:6;
   (c) a heavy chain CDR3 having at least 90% sequence identity to the amino acid sequence of SEQ ID NO:7 or SEQ ID NO:8 or having up to two amino acid substitutions relative to the amino acid sequence of SEQ ID NO:7 or SEQ ID NO:8;
   (d) a light chain CDR1 having at least 90% sequence identity to the amino acid sequence of SEQ ID NO:11 or having up to two amino acid substitutions relative to the amino acid sequence of SEQ ID NO:11;
   (e) a light chain CDR2 having up to two amino acid substitutions relative to the amino acid sequence of SEQ ID NO:12; and
   (f) a light chain CDR3 comprising the amino acid sequence of SEQ ID NO:13 or 14.
25 The isolated antibody of embodiment 24, wherein the antigen binding site comprises a heavy chain variable region comprising an amino acid sequence having at least 90% sequence identity to any one of SEQ ID NOS:1-3 and a light chain variable region comprising an amino acid sequence having at least 90% sequence identity to any one of SEQ ID NOS:9-10.
26. The isolated antibody of embodiment 24, wherein the antigen binding site comprises a heavy chain variable region comprising the amino acid sequence of any one of SEQ ID NOS:1-3 and a light chain variable region comprising the amino acid sequence of any one of SEQ ID NOS:9-10.
27. The isolated antibody of any one of embodiments 23-26, further comprising a second antigen binding site comprising one or more CDRs selected from the group consisting of:
   (a) a heavy chain CDR1 comprising the amino acid sequence of SEQ ID NO:16 or having up to two amino acid substitutions relative to the amino acid sequence of SEQ ID NO:16;
   (b) a heavy chain CDR2 having at least 90% sequence identity to the amino acid sequence of SEQ ID NO:17 or having up to two amino acid substitutions relative to the amino acid sequence of SEQ ID NO:17; and
   (c) a heavy chain CDR3 having at least 90% sequence identity to the amino acid sequence of SEQ ID NO:18 or having up to two amino acid substitutions relative to the amino acid sequence of SEQ ID NO:18.
28. The isolated antibody of embodiment 27, wherein the second antigen binding site comprises a heavy chain variable region comprising an amino acid sequence having at least 90% sequence identity to SEQ ID NO:15.
29. The isolated antibody of embodiment 27 or 28, wherein the second antigen binding site further comprises one or more CDRs selected from the group consisting of:
   (a) a light chain CDR1 having at least 90% sequence identity to the amino acid sequence of SEQ ID NO:11 or having up to two amino acid substitutions relative to the amino acid sequence of SEQ ID NO:11;
   (b) a light chain CDR2 having up to two amino acid substitutions relative to the amino acid sequence of SEQ ID NO:12; and
   (c) a light chain CDR3 having up to two amino acid substitutions relative to the amino acid sequence of SEQ ID NO:13 or 14.
30. The isolated antibody of embodiment 29, wherein the second antigen binding site comprises a light chain variable region comprising an amino acid sequence having at least 90% sequence identity to any one of SEQ ID NOS:9-10.
31. The isolated antibody of embodiment 29 or 30, wherein the first and second antigen binding sites comprise the same light chain CDR1, CDR2, and CDR3 sequences.
32. The isolated antibody of embodiment 31, comprising heavy and light chain CDRs selected from the combinations listed in Table 1.
33. An isolated antibody comprising heavy and light chains selected from the combinations listed in Table 2.
34. An isolated antibody comprising:
   (a) a first antigen binding site for human epidermal growth factor receptor 2 (HER2) subdomain IV;
   (b) a second antigen binding site for human HER2 subdomain II; and
   (c) a modified Fc polypeptide dimer comprising a first Fc polypeptide that contains modifications that create a TfR-binding site, wherein a light chain polypeptide sequence in the first antigen binding site is identical to a light chain polypeptide sequence in the second antigen binding site.
35. The isolated antibody of embodiment 34, wherein the first Fc polypeptide comprises a modified CH3 domain comprising the TfR-binding site.
36. The isolated antibody of embodiment 35, wherein the modified CH3 domain is derived from a human IgG1, IgG2, IgG3, or IgG4 CH3 domain.
37. The isolated antibody of embodiment 35 or 36, wherein the modified CH3 domain comprises one, two, three, four, five, six, seven, eight, nine, ten, or eleven substitutions in a set of amino acid positions comprising 380, 384, 386, 387, 388, 389, 390, 413, 415, 416, and 421, according to EU numbering.
38. The isolated antibody of any one of embodiments 35 to 37, wherein the modified CH3 domain comprises Glu, Leu, Ser, Val, Trp, Tyr, or Gln at position 380; Leu, Tyr, Phe, Trp, Met, Pro, or Val at position 384; Leu, Thr, His, Pro, Asn, Val, or Phe at position 386; Val, Pro, Ile, or an acidic amino acid at position 387; Trp at position 388; an aliphatic amino acid, Gly, Ser, Thr, or Asn at position 389; Gly, His, Gln, Leu, Lys, Val, Phe, Ser, Ala, Asp, Glu, Asn, Arg, or Thr at position 390; an acidic amino acid, Ala, Ser, Leu, Thr, Pro, Ile, or His at position 413; Glu, Ser, Asp, Gly, Thr, Pro, Gln, or Arg at position 415; Thr, Arg, Asn, or an acidic amino acid at position 416; and/or an aromatic amino acid, His, or Lys at position 421, according to EU numbering.
39. The isolated antibody of any one of embodiments 34 to 38, wherein the first Fc polypeptide that contains modifications that create the TfR-binding site binds to the apical domain of TfR.
40. The isolated antibody of any one of embodiments 34 to 39, wherein the first Fc polypeptide and the second Fc polypeptide each comprises modifications that promote heterodimerization.
41. The isolated antibody of embodiment 40, wherein the first Fc polypeptide comprises a T366W substitution and the second Fc polypeptide comprises T366S, L368A, and Y407V substitutions, according to EU numbering.
42. The isolated antibody of embodiment 40, wherein the first Fc polypeptide comprises T366S, L368A, and Y407V substitutions and the second Fc polypeptide comprises a T366W substitution, according to EU numbering.
43. The isolated antibody of any one of embodiments 34 to 42, wherein the first Fc polypeptide and/or the second Fc polypeptide independently comprises modifications that reduce TfR-mediated effector function.
44. The isolated antibody of embodiment 43, wherein the modifications that reduce effector function are L234A and L235A substitutions, according to EU numbering.
45. The isolated antibody of embodiment 44, wherein the first Fc polypeptide specifically binds to TfR and comprises L234A and L235A substitutions.
46. The isolated antibody of embodiment 45, wherein the first Fc polypeptide further comprises a P329G or a P329S substitution, according to EU numbering.
47. The isolated antibody of embodiment 46, wherein the second Fc polypeptide comprises Leu at positions 234 and 235 and a proline at position 329, according to EU numbering.
48. The isolated antibody of embodiment 44, wherein the second Fc polypeptide specifically binds to TfR and comprises L234A and L235A substitutions.
49. The isolated antibody of embodiment 48, wherein the second Fc polypeptide further comprises a P329G or a P329S substitution, according to EU numbering.
50. The isolated antibody of embodiment 49, wherein the first Fc polypeptide comprises Leu at positions 234 and 235 and a proline at position 329, according to EU numbering.
51. The isolated antibody of any one of embodiments 34 to 50, wherein a hinge region or a portion thereof is linked to the N-terminus of the first Fc polypeptide and/or the second Fc polypeptide.
52. The isolated antibody of any one of embodiments 34 to 51, wherein the first Fc polypeptide and/or the second Fc polypeptide independently comprises a sequence having at least 90% identity to a sequence selected from the group consisting of SEQ ID NOS:71-86 and 98-100.
53. The isolated antibody of embodiment 52, wherein the first Fc polypeptide or the second Fc polypeptide comprises a sequence having at least 90% identity to a sequence selected from the group consisting of SEQ ID NOS:71-73, 85, and 99-100.
54. The isolated antibody of embodiment 52, wherein the first Fc polypeptide or the second Fc polypeptide comprises a sequence having at least 90% identity to a sequence selected from the group consisting of SEQ ID NOS:74-84, 86 and 98.
55. The isolated antibody of embodiment 34, wherein:
   the first antigen binding site comprises the amino acid sequence of SEQ ID NO:15;
   the second antigen binding site comprises an amino acid sequence selected from the group consisting of SEQ ID NOS:1-3 and 60-70;
   the first Fc polypeptide that contains modifications that create the TfR-binding site comprises an amino acid sequence selected from the group consisting of SEQ ID NOS:74-84, 86, and 98; and
   the light chain polypeptide sequence comprises the amino acid sequence of SEQ ID NO:9 or SEQ ID NO:10.
56. The isolated antibody of embodiment 55, further comprising a second Fc polypeptide comprising an amino acid sequence selected from the group consisting of SEQ ID NOS:71-73, 85, and 99-100.
57. The isolated antibody of any one of embodiments 34 to 56, wherein the first Fc polypeptide and/or the second Fc polypeptide independently comprises a S239D and/or a I332E substitution, according to EU numbering.
58. The isolated antibody of embodiment 57, wherein the first Fc polypeptide and/or the second Fc polypeptide independently comprising the S239D and/or the I332E substitution is capable of enhancing HER2-mediated effector function.
59. The isolated antibody of embodiment 57 or 58, wherein:
   (a) the first Fc polypeptide comprises a S239D substitution and the second Fc polypeptide comprises a S239D substitution, according to EU numbering;
   (b) the first Fc polypeptide comprises a I332E substitution and the second Fc polypeptide comprises a S239D substitution, according to EU numbering;
   (c) the first Fc polypeptide comprises a S239D and a I332E substitution and the second Fc polypeptide comprises a S239D substitution, according to EU numbering;
   (d) the second Fc polypeptide comprises a S239D substitution, according to EU numbering;
   (e) the first Fc polypeptide comprises a S239D substitution and the second Fc polypeptide comprises a I332E substitution, according to EU numbering;
   (f) the first Fc polypeptide comprises a I332E substitution and the second Fc polypeptide comprises a I332E substitution, according to EU numbering;
   (g) the first Fc polypeptide comprises a S239D and a I332E substitution and the second Fc polypeptide comprises a I332E substitution, according to EU numbering;
   (h) the second Fc polypeptide comprises a I332E substitution, according to EU numbering;
   (i) the first Fc polypeptide comprises a S239D substitution and the second Fc polypeptide comprises a S239D and a I332E substitution, according to EU numbering;
   (j) the first Fc polypeptide comprises a I332E substitution and the second Fc polypeptide comprises a S239D and a I332E substitution, according to EU numbering;
   (k) the first Fc polypeptide comprises a S239D and a I332E substitution and the second Fc polypeptide comprises a S239D and a I332E substitution, according to EU numbering;
   (l) the second Fc polypeptide comprises a S239D and a I332E substitution, according to EU numbering;
   (m) the first Fc polypeptide comprises a S239D substitution, according to EU numbering;
   (n) the first Fc polypeptide comprises a I332E substitution, according to EU numbering; or
   (o) the first Fc polypeptide comprises a S239D and a I332E substitution, according to EU numbering.
60. The isolated antibody of embodiment 59, wherein:
   (a) the first Fc polypeptide comprises a I332E substitution and the second Fc polypeptide comprises a S239D substitution, according to EU numbering;
   (b) the first Fc polypeptide comprises a S239D and a I332E substitution and the second Fc polypeptide comprises a S239D substitution, according to EU numbering;
   (c) the first Fc polypeptide comprises a S239D substitution and the second Fc polypeptide comprises a I332E substitution, according to EU numbering;
   (d) the second Fc polypeptide comprises a I332E substitution, according to EU numbering;
   (e) the first Fc polypeptide comprises a S239D substitution and the second Fc polypeptide comprises a S239D and a I332E substitution, according to EU numbering; or
   (f) the first Fc polypeptide comprises a I332E substitution, according to EU numbering.
61. The isolated antibody of embodiment 60, wherein:
   (a) the first Fc polypeptide comprises a I332E substitution and a serine at position 239, and the second Fc polypeptide comprises a S239D substitution and a isoleucine at position 332, according to EU numbering;
   (b) the first Fc polypeptide comprises a S239D and a I332E substitution, and the second Fc polypeptide comprises a S239D substitution and a isoleucine at position 332, according to EU numbering;
   (c) the first Fc polypeptide comprises a S239D substitution and a isoleucine at position 332, and the second Fc polypeptide comprises a I332E substitution and a serine at position 239, according to EU numbering;
   (d) the first Fc polypeptide comprises a serine at position 239 and a isoleucine at 332, and the second Fc polypeptide comprises a I332E substitution and a serine at position 239, according to EU numbering;
   (e) the first Fc polypeptide comprises a S239D substitution and a isoleucine at position 332, and the second Fc polypeptide comprises a S239D and a I332E substitution, according to EU numbering; or
   (f) the first Fc polypeptide comprises a I332E substitution and a serine at position 239, according to EU numbering, and the second Fc polypeptide comprises a serine at position 239 and a isoleucine at 332.
62. The isolated antibody of any one of embodiments 34 to 61, comprising two heavy chains and two light chains.
63. The isolated antibody of embodiment 62, comprising heavy and light chains selected from the combinations listed in Table 2.
64. The isolated antibody of embodiment 62, wherein the first heavy chain comprises a V_{H} and a Fc sequence selected from the combinations in Table 3 and the second heavy chain comprises a V_{H} and a Fc sequence selected from the combinations in Table 4.
65. The isolated antibody of embodiment 62, wherein the first heavy chain comprises a V_{H} and a Fc sequence selected from the combinations in Table 5 and the second heavy chain comprises a V_{H} and a Fc sequence selected from the combinations in Table 6.
66. A pharmaceutical composition comprising the isolated antibody of any one of embodiments 1 to 65 and a pharmaceutically acceptable carrier.
67. An isolated polynucleotide comprising a nucleotide sequence encoding the isolated antibody of any one of embodiments 1 to 65.
68. A vector comprising the polynucleotide of embodiment 67.
69. A host cell comprising the polynucleotide of embodiment 67 or the vector of embodiment 68.
70. A method for treating a cancer or treating brain metastasis of a cancer in a subject, the method comprising administering to the subject a therapeutically effective amount of the isolated antibody of any one of embodiments 1 to 65 or the pharmaceutical composition of embodiment 66.
71. The method of embodiment 70, wherein the isolated antibody is adminstered in combination with a chemotherapy or radiation therapy.
72. The method of embodiment 70 or 71, wherein the cancer is a metastatic cancer.
73. The method of any one of embodiments 70 to 72, wherein the cancer is a breast cancer.
74. The method of any one of embodiments 70 to 73, wherein the cancer is a HER2-positive cancer.

It is understood that the examples and embodiments described herein are for illustrative purposes only and that various modifications or changes in light thereof will be suggested to persons skilled in the art and are to be included within the spirit and purview of this application and scope of the appended claims. The sequences of the sequence accession numbers cited herein are hereby incorporated by reference.

TABLE 1
CDR combinations

| | HC1_D4 | HC1_D4 | HC1_D4 | HC2_D2 | HC2_D2 | HC2_D2 | LC | LC | LC |
|---|---|---|---|---|---|---|---|---|---|
| | CDR1 | CDR2 | CDR3 | CDR1 | CDR2 | CDR3 | CDR1 | CDR2 | CDR3 |
| | SEQ ID | SEQ ID | SEQ ID | SEQ ID | SEQ ID | SEQ ID | SEQ ID | SEQ ID | SEQ ID |
| Combo # | NO | NO | NO | NO | NO | NO | NO | NO | NO |
| A | 16 | 17 | 18 | 4 | 6 | 7 | 11 | 12 | 13 |
| B | 16 | 17 | 18 | 4 | 5 | 8 | 11 | 12 | 13 |
| C | 16 | 17 | 18 | 4 | 6 | 8 | 11 | 12 | 13 |
| D | 16 | 17 | 18 | 4 | 6 | 7 | 11 | 12 | 14 |
| E | 16 | 17 | 18 | 4 | 5 | 8 | 11 | 12 | 14 |
| F | 16 | 17 | 18 | 4 | 6 | 8 | 11 | 12 | 14 |
| G | 16 | 17 | 18 | 49 | 5 | 7 | 11 | 12 | 13 |
| H | 16 | 17 | 18 | 50 | 5 | 7 | 11 | 12 | 13 |
| I | 16 | 17 | 18 | 51 | 5 | 7 | 11 | 12 | 13 |
| J | 16 | 17 | 18 | 52 | 5 | 7 | 11 | 12 | 13 |
| K | 16 | 17 | 18 | 4 | 53 | 7 | 11 | 12 | 13 |
| L | 16 | 17 | 18 | 4 | 54 | 7 | 11 | 12 | 13 |
| M | 16 | 17 | 18 | 4 | 55 | 7 | 11 | 12 | 13 |
| N | 16 | 17 | 18 | 4 | 5 | 56 | 11 | 12 | 13 |
| O | 16 | 17 | 18 | 4 | 5 | 57 | 11 | 12 | 13 |
| P | 16 | 17 | 18 | 4 | 5 | 58 | 11 | 12 | 13 |
| Q | 16 | 17 | 18 | 4 | 5 | 59 | 11 | 12 | 13 |
| R | 16 | 17 | 18 | 49 | 5 | 7 | 11 | 12 | 14 |
| S | 16 | 17 | 18 | 50 | 5 | 7 | 11 | 12 | 14 |
| T | 16 | 17 | 18 | 51 | 5 | 7 | 11 | 12 | 14 |
| U | 16 | 17 | 18 | 52 | 5 | 7 | 11 | 12 | 14 |
| V | 16 | 17 | 18 | 4 | 53 | 7 | 11 | 12 | 14 |
| W | 16 | 17 | 18 | 4 | 54 | 7 | 11 | 12 | 14 |
| X | 16 | 17 | 18 | 4 | 55 | 7 | 11 | 12 | 14 |
| Y | 16 | 17 | 18 | 4 | 5 | 56 | 11 | 12 | 14 |
| Z | 16 | 17 | 18 | 4 | 5 | 57 | 11 | 12 | 14 |
| AB | 16 | 17 | 18 | 4 | 5 | 58 | 11 | 12 | 14 |
| AC | 16 | 17 | 18 | 4 | 5 | 59 | 11 | 12 | 14 |

TABLE 2
Heavy chain (HC) and light chain (LC) combinations

| Combo | HC1_D4 | HC2_D2 | |
|---|---|---|---|
| # | SEQ ID NO | SEQ ID NO | LC SEQ ID NO |
| A | 37 | 25 | 9 |
| B | 31 | 30 | 9 |
| C | 31 | 29 | 9 |
| D | 38 | 30 | 9 |
| E | 32 | 30 | 9 |
| F | 32 | 29 | 9 |
| G | 39 | 30 | 9 |
| H | 37 | 26 | 9 |
| I | 31 | 34 | 9 |
| J | 31 | 33 | 9 |
| K | 38 | 34 | 9 |
| L | 32 | 34 | 9 |
| M | 32 | 33 | 9 |
| N | 39 | 34 | 9 |
| O | 37 | 27 | 9 |
| P | 31 | 36 | 9 |
| Q | 31 | 35 | 9 |
| R | 38 | 36 | 9 |
| S | 32 | 36 | 9 |
| T | 32 | 35 | 9 |
| U | 39 | 46 | 9 |
| V | 37 | 25 | 10 |
| W | 31 | 30 | 10 |
| X | 31 | 29 | 10 |
| Y | 38 | 30 | 10 |
| Z | 32 | 30 | 10 |
| AA | 32 | 29 | 10 |
| AB | 39 | 30 | 10 |
| AC | 37 | 26 | 10 |
| AD | 31 | 34 | 10 |
| AE | 31 | 33 | 10 |
| AF | 38 | 34 | 10 |
| AG | 32 | 34 | 10 |
| AH | 32 | 33 | 10 |
| AI | 39 | 34 | 10 |
| AJ | 37 | 27 | 10 |
| AK | 31 | 36 | 10 |
| AL | 31 | 35 | 10 |
| AM | 38 | 36 | 10 |
| AN | 32 | 36 | 10 |
| AO | 32 | 35 | 10 |
| AP | 39 | 46 | 10 |
| AQ | 20 | 24 | 19 |
| AR | 21 | 24 | 19 |
| AS | 22 | 24 | 19 |
| AT | 23 | 24 | 19 |

TABLE 3
HC_D2 V_{H} and Fc (hole) combinations

| Combo # | V_{H}_D2 SEQ ID NO | Fc SEQ ID NO |
|---|---|---|
| A | 1 | 71 |
| B | 2 | 71 |
| C | 3 | 71 |
| D | 1 | 72 |
| E | 2 | 72 |
| F | 3 | 72 |
| G | 1 | 73 |
| H | 2 | 73 |
| I | 3 | 73 |
| J | 1 | 85 |
| K | 2 | 85 |
| L | 3 | 85 |

TABLE 4
HC_D4 V_{H} and Fc (knob) combinations

| Combo # | V_{H}_D4 SEQ ID NO | Fc SEQ ID NO |
|---|---|---|
| A | 15 | 86 |
| B | 15 | 74 |
| C | 15 | 75 |
| D | 15 | 76 |
| E | 15 | 77 |
| F | 15 | 78 |
| G | 15 | 79 |
| H | 15 | 80 |
| I | 15 | 81 |
| J | 15 | 82 |
| K | 15 | 83 |
| L | 15 | 84 |

TABLE 5
HC_D2 V_{H} and Fc (knob) combinations

| Combo # | V_{H}_D2 SEQ ID NO | Fc SEQ ID NO |
|---|---|---|
| A | 1 | 86 |
| B | 1 | 74 |
| C | 1 | 75 |
| D | 1 | 76 |
| E | 1 | 77 |
| F | 1 | 78 |
| G | 1 | 79 |
| H | 1 | 80 |
| I | 1 | 81 |
| J | 1 | 82 |
| K | 1 | 83 |
| L | 1 | 84 |
| M | 2 | 86 |
| N | 2 | 74 |
| O | 2 | 75 |
| P | 2 | 76 |
| Q | 2 | 77 |
| R | 2 | 78 |
| S | 2 | 79 |
| T | 2 | 80 |
| U | 2 | 81 |
| V | 2 | 82 |
| W | 2 | 83 |
| X | 2 | 84 |
| Y | 2 | 86 |
| Z | 2 | 74 |
| AA | 2 | 75 |
| AB | 3 | 76 |
| AC | 3 | 77 |
| AD | 3 | 78 |
| AE | 3 | 79 |
| AF | 3 | 80 |
| AG | 3 | 81 |
| AH | 3 | 82 |
| AI | 3 | 83 |
| AJ | 3 | 84 |

TABLE 6
HC_D4 V_{H} and Fc (hole) combinations

| Combo # | V_{H}_D4 SEQ ID NO | Fc SEQ ID NO |
|---|---|---|
| A | 15 | 71 |
| B | 15 | 72 |
| C | 15 | 73 |
| D | 15 | 85 |

INFORMAL SEQUENCE LISTING

| SEQ | | |
|---|---|---|
| ID | | |
| NO | Sequence | Description |
| 1 | EVQLVESGGGLVQPGGSLRLSCAASGFTFTDYTMDWVRQAP | Anti-HER2_D2_VH_v1 |
| | GKGLEWVADVNPNSGQSIYNQRFKGRFTLSVDRSKNTLYLQ | |
| | MNSLRAEDTAVYYCARNLGPSFYFDYWGQGTLVTVSS | |
| 2 | EVQLVESGGGLVQPGGSLRLSCAASGFTFTDYTMDWVRQAP | Anti-HER2_D2_VH_v2 |
| | GKGLEWVADVNPNSGGSIYNQRFKGRFTLSVDRSKNTLYLQ | |
| | MNSLRAEDTAVYYCARNLGPLFYFDYWGQGTLVTVSS | |
| 3 | EVQLVESGGGLVQPGGSLRLSCAASGFTFTDYTMDWVRQAP | Anti-HER2_D2_VH_v3 |
| | GKGLEWVADVNPNSGQSIYNQRFKGRFTLSVDRSKNTLYLQ | |
| | MNSLRAEDTAVYYCARNLGPLFYFDYWGQGTLVTVSS | |
| 4 | GFTFTDYTMD | Anti-HER2_D2_CDR-H1 |
| 5 | DVNPNSGGSIYNQRFKG | Anti-HER2_D2_CDR-H2 |
| 6 | DVNPNSGQSIYNQRFKG | Anti-HER2_D2_CDR-H2.1 |
| 7 | ARNLGPSFYFDY | Anti-HER2_D2_CDR-H3 |
| 8 | ARNLGPLFYFDY | Anti-HER2_D2_CDR-H3.1 |
| 9 | DIQMTQSPSSLSASVGDRVTITCRASQDVNTAVAWYQQKPGK | Anti-HER2_light chain_v1 |
| | APKLLIYSASFLYSGVPSRFSGSRSGTDFTLTISSLQPEDFATYY | |
| | CQQFYTTPPTFGQGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| 10 | DIQMTQSPSSLSASVGDRVTITCRASQDVNTAVAWYQQKPGK | Anti-HER2_light chain_v2 |
| | APKLLIYSASFLYSGVPSRFSGSRSGTDFTLTISSLQPEDFATYY | |
| | CQQYYTTPPTFGQGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| 11 | RASQDVNTAVA | Anti-HER2_CDR-L1 |
| 12 | SASFLYS | Anti-HER2_CDR-L2 |
| 13 | QQFYTTPPT | Anti-HER2_CDR-L3.1 |
| 14 | QQYYTTPPT | Anti-HER2_CDR-L3.2 |
| 15 | EVQLVESGGGLVQPGGSLRLSCAASGFNIKDTYIHWVRQAPG | Anti-HER2_D4_VH |
| | KGLEWVARIYPTNGYTRYADSVKGRFTISADTSKNTAYLQM | |
| | NSLRAEDTAVYYCSRWGGDGFYAMDYWGQGTLVTVSSAST | |
| | KGPSVFPLAPSSKSTSGGTAALGCLVKDYFPEPVTVSWNSGA | |
| | LTSGVHTFPAVLQSS | |
| 16 | GFNIKDTYIH | Anti-HER2_D4_CDR-H1 |
| 17 | RIYPTNGYTRYADSVKG | Anti-HER2_D4_CDR-H2 |
| 18 | RWGGDGFYAMDY | Anti-HER2_D4_CDR-H3 |
| 19 | DIQMTQSPSSLSASVGDRVTITCKASQDVSTAVAWYQQKPGK | Anti-HER2_light chain_v3 |
| | APKLLIYSASFRYTGVPSRFSGSRSGTDFTLTISSLQPEDFATY | |
| | YCQQHYTTPPTFGQGTKVEIKRTVAAPSVFIFPPSDEQLKSGT | |
| | ASVVCLLNNFYPREAKVQWKVDNALQSGNSQESVTEQDSKD | |
| | STYSLSSTLTLSKADYEKHKVYACEVTHQGLSSPVTKSFNRG | |
| | EC | |
| 20 | EVQLVESGGGLVQPGGSLRLSCAASGFNIKDTYIHWVRQAPG | Anti-HER2_D4_heavy chain_ |
| | KGLEWVARIYPTNGYTRYADSVKGRFTISADTSKNTAYLQM | v1 |
| | NSLRAEDTAVYYCSRWGGEGFYAMDYWGQGTLVTVSSAST | |
| | KGPSVFPLAPSSKSTSGGTAALGCLVKDYFPEPVTVSWNSGA | |
| | LTSGVHTFPAVLQSSGLYSLSSVVTVPSSSLGTQTYICNVNHK | |
| | PSNTKVDKKVEPKSCDKTHTCPPCPAPELLGGPSVFLFPPKPK | |
| | DTLMISRTPEVTCVVVDVSHEDPEVKFNWYVDGVEVHNAKT | |
| | KPREEQYNSTYRVVSVLTVLHQDWLNGKEYKCKVSNKALPA | |
| | PIEKTISKAKGQPREPQVYTLPPSRDELTKNQVSLWCLVKGFY | |
| | PSDIAVEWESNGQPENNYKTTPPVLDSDGSFFLYSKLTVDKSR | |
| | WQQGNVFSCSVMHEALHNHYTQKSLSLSPGK | |
| 21 | EVQLVESGGGLVQPGGSLRLSCAASGFNIKDTYIHWVRQAPG | Anti-HER2_D4_heavy chain |
| | KGLEWVARIYPTNGYTRYADSVKGRFTISADTSKNTAYLQM | v2 |
| | NSLRAEDTAVYYCSRWGGEGFYAMDYWGQGTLVTVSSAST | |
| | KGPSVFPLAPSSKSTSGGTAALGCLVKDYFPEPVTVSWNSGA | |
| | LTSGVHTFPAVLQSSGLYSLSSVVTVPSSSLGTQTYICNVNHK | |
| | PSNTKVDKKVEPKSCDKTHTCPPCPAPEAAGGPSVFLFPPKPK | |
| | DTLMISRTPEVTCVVVDVSHEDPEVKFNWYVDGVEVHNAKT | |
| | KPREEQYNSTYRVVSVLTVLHQDWLNGKEYKCKVSNKALPA | |
| | PIEKTISKAKGQPREPQVYTLPPSRDELTKNQVSLWCLVKGFY | |
| | PSDIAVEWESYGTEWSNYKTTPPVLDSDGSFFLYSKLTVSKEE | |
| | WQQGFVFSCSVMHEALHNHYTQKSLSLSPGK | |
| 22 | EVQLVESGGGLVQPGGSLRLSCAASGFNIKDTYIHWVRQAPG | Anti-HER2_D4_heavy |
| | KGLEWVARIYPTNGYTRYADSVKGRFTISADTSKNTAYLQM | chain_v3 |
| | NSLRAEDTAVYYCSRWGGEGFYAMDYWGQGTLVTVSSAST | |
| | KGPSVFPLAPSSKSTSGGTAALGCLVKDYFPEPVTVSWNSGA | |
| | LTSGVHTFPAVLQSSGLYSLSSVVTVPSSSLGTQTYICNVNHK | |
| | PSNTKVDKKVEPKSCDKTHTCPPCPAPEAAGGPSVFLFPPKPK | |
| | DTLMISRTPEVTCVVVDVSHEDPEVKFNWYVDGVEVHNAKT | |
| | KPREEQYNSTYRVVSVLTVLHQDWLNGKEYKCKVSNKALG | |
| | APIEKTISKAKGQPREPQVYTLPPSRDELTKNQVSLWCLVKGF | |
| | YPSDIAVEWESYGTEWSNYKTTPPVLDSDGSFFLYSKLTVSKE | |
| | EWQQGFVFSCSVMHEALHNHYTQKSLSLSPGK | |
| 23 | EVQLVESGGGLVQPGGSLRLSCAASGFNIKDTYIHWVRQAPG | Anti-HER2_D4_heavy |
| | KGLEWVARIYPTNGYTRYADSVKGRFTISADTSKNTAYLQM | chain_v4 |
| | NSLRAEDTAVYYCSRWGGEGFYAMDYWGQGTLVTVSSAST | |
| | KGPSVFPLAPSSKSTSGGTAALGCLVKDYFPEPVTVSWNSGA | |
| | LTSGVHTFPAVLQSSGLYSLSSVVTVPSSSLGTQTYICNVNHK | |
| | PSNTKVDKKVEPKSCDKTHTCPPCPAPEAAGGPSVFLFPPKPK | |
| | DTLMISRTPEVTCVVVDVSHEDPEVKFNWYVDGVEVHNAKT | |
| | KPREEQYNSTYRVVSVLTVLHQDWLNGKEYKCKVSNKALSA | |
| | PIEKTISKAKGQPREPQVYTLPPSRDELTKNQVSLWCLVKGFY | |
| | PSDIAVEWESYGTEWSNYKTTPPVLDSDGSFFLYSKLTVSKEE | |
| | WQQGFVFSCSVMHEALHNHYTQKSLSLSPGK | |
| 24 | EVQLVESGGGLVQPGGSLRLSCAASGFTFNDYTMDWVRQAP | Anti-HER2_D2 heavy |
| | GKGLEWVADVNPNSGGSIVNRRFKGRFTLSVDRSKNTLYLQ | chain_v1 |
| | MNSLRAEDTAVYYCARNLGPFFYFDYWGQGTLVTVSSASTK | |
| | GPSVFPLAPSSKSTSGGTAALGCLVKDYFPEPVTVSWNSGALT | |
| | SGVHTFPAVLQSSGLYSLSSVVTVPSSSLGTQTYICNVNHKPS | |
| | NTKVDKKVEPKSCDKTHTCPPCPAPELLGGPSVFLFPPKPKDT | |
| | LMISRTPEVTCVVVDVSHEDPEVKFNWYVDGVEVHNAKTKP | |
| | REEQYNSTYRVVSVLTVLHQDWLNGKEYKCKVSNKALPAPI | |
| | EKTISKAKGQPREPQVYTLPPSRDELTKNQVSLSCAVKGFYPS | |
| | DIAVEWESNGQPENNYKTTPPVLDSDGSFFLVSKLTVDKSRW | |
| | QQGNVFSCSVMHEALHNHYTQKSLSLSPGK | |
| 25 | EVQLVESGGGLVQPGGSLRLSCAASGFTFTDYTMDWVRQAP | Anti-HER2_D2 heavy |
| | GKGLEWVADVNPNSGQSIYNQRFKGRFTLSVDRSKNTLYLQ | chain_v2 |
| | MNSLRAEDTAVYYCARNLGPSFYFDYWGQGTLVTVSSASTK | |
| | GPSVFPLAPSSKSTSGGTAALGCLVKDYFPEPVTVSWNSGALT | |
| | SGVHTFPAVLQSSGLYSLSSVVTVPSSSLGTQTYICNVNHKPS | |
| | NTKVDKKVEPKSCDKTHTCPPCPAPELLGGPSVFLFPPKPKDT | |
| | LMISRTPEVTCVVVDVSHEDPEVKFNWYVDGVEVHNAKTKP | |
| | REEQYNSTYRVVSVLTVLHQDWLNGKEYKCKVSNKALPAPI | |
| | EKTISKAKGQPREPQVYTLPPSRDELTKNQVSLSCAVKGFYPS | |
| | DIAVEWESNGQPENNYKTTPPVLDSDGSFFLVSKLTVDKSRW | |
| | QQGNVFSCSVMHEALHNHYTQKSLSLSPGK | |
| 26 | EVQLVESGGGLVQPGGSLRLSCAASGFTFTDYTMDWVRQAP | Anti-HER2_D2 heavy |
| | GKGLEWVADVNPNSGGSIYNQRFKGRFTLSVDRSKNTLYLQ | chain v3 |
| | MNSLRAEDTAVYYCARNLGPLFYFDYWGQGTLVTVSSASTK | |
| | GPSVFPLAPSSKSTSGGTAALGCLVKDYFPEPVTVSWNSGALT | |
| | SGVHTFPAVLQSSGLYSLSSVVTVPSSSLGTQTYICNVNHKPS | |
| | NTKVDKKVEPKSCDKTHTCPPCPAPELLGGPSVFLFPPKPKDT | |
| | LMISRTPEVTCVVVDVSHEDPEVKFNWYVDGVEVHNAKTKP | |
| | REEQYNSTYRVVSVLTVLHQDWLNGKEYKCKVSNKALPAPI | |
| | EKTISKAKGQPREPQVYTLPPSRDELTKNQVSLSCAVKGFYPS | |
| | DIAVEWESNGQPENNYKTTPPVLDSDGSFFLVSKLTVDKSRW | |
| | QQGNVFSCSVMHEALHNHYTQKSLSLSPGK | |
| 27 | EVQLVESGGGLVQPGGSLRLSCAASGFTFTDYTMDWVRQAP | Anti-HER2_D2_heavy |
| | GKGLEWVADVNPNSGQSIYNQRFKGRFTLSVDRSKNTLYLQ | chain_v4 |
| | MNSLRAEDTAVYYCARNLGPLFYFDYWGQGTLVTVSSASTK | |
| | GPSVFPLAPSSKSTSGGTAALGCLVKDYFPEPVTVSWNSGALT | |
| | SGVHTFPAVLQSSGLYSLSSVVTVPSSSLGTQTYICNVNHKPS | |
| | NTKVDKKVEPKSCDKTHTCPPCPAPELLGGPSVFLFPPKPKDT | |
| | LMISRTPEVTCVVVDVSHEDPEVKFNWYVDGVEVHNAKTKP | |
| | REEQYNSTYRVVSVLTVLHQDWLNGKEYKCKVSNKALPAPI | |
| | EKTISKAKGQPREPQVYTLPPSRDELTKNQVSLSCAVKGFYPS | |
| | DIAVEWESNGQPENNYKTTPPVLDSDGSFFLVSKLTVDKSRW | |
| | QQGNVFSCSVMHEALHNHYTQKSLSLSPGK | |
| 28 | EVQLVESGGGLVQPGGSLRLSCAASGFNIKDTYIHWVRQAPG | Anti-HER2_D4_heavy |
| | KGLEWVARIYPTNGYTRYADSVKGRFTISADTSKNTAYLQM | chain_v5 |
| | NSLRAEDTAVYYCSRWGGDGFYAMDYWGQGTLVTVSSAST | |
| | KGPSVFPLAPSSKSTSGGTAALGCLVKDYFPEPVTVSWNSGA | |
| | LTSGVHTFPAVLQSSGLYSLSSVVTVPSSSLGTQTYICNVNHK | |
| | PSNTKVDKKVEPKSCDKTHTCPPCPAPEAAGGPDVFLFPPKPK | |
| | DTLMISRTPEVTCVVVDVSHEDPEVKFNWYVDGVEVHNAKT | |
| | KPREEQYNSTYRVVSVLTVLHQDWLNGKEYKCKVSNKALPA | |
| | PIEKTISKAKGQPREPQVYTLPPSRDELTKNQVSLWCLVKGFY | |
| | PSDIAVEWESYGTEWSNYKTTPPVLDSDGSFFLYSKLTVSKEE | |
| | WQQGFVFSCSVMHEALHNHYTQKSLSLSPGK | |
| 29 | EVQLVESGGGLVQPGGSLRLSCAASGFTFTDYTMDWVRQAP | Anti-HER2_D2_heavy |
| | GKGLEWVADVNPNSGQSIYNQRFKGRFTLSVDRSKNTLYLQ | chain_v5 |
| | MNSLRAEDTAVYYCARNLGPSFYFDYWGQGTLVTVSSASTK | |
| | GPSVFPLAPSSKSTSGGTAALGCLVKDYFPEPVTVSWNSGALT | |
| | SGVHTFPAVLQSSGLYSLSSVVTVPSSSLGTQTYICNVNHKPS | |
| | NTKVDKKVEPKSCDKTHTCPPCPAPELLGGPDVFLFPPKPKDT | |
| | LMISRTPEVTCVVVDVSHEDPEVKFNWYVDGVEVHNAKTKP | |
| | REEQYNSTYRVVSVLTVLHQDWLNGKEYKCKVSNKALPAPE | |
| | EKTISKAKGQPREPQVYTLPPSRDELTKNQVSLSCAVKGFYPS | |
| | DIAVEWESNGQPENNYKTTPPVLDSDGSFFLVSKLTVDKSRW | |
| | QQGNVFSCSVMHEALHNHYTQKSLSLSPGK | |
| 30 | EVQLVESGGGLVQPGGSLRLSCAASGFTFTDYTMDWVRQAP | Anti-HER2_D2_heavy |
| | GKGLEWVADVNPNSGQSIYNQRFKGRFTLSVDRSKNTLYLQ | chain_v6 |
| | MNSLRAEDTAVYYCARNLGPSFYFDYWGQGTLVTVSSASTK | |
| | GPSVFPLAPSSKSTSGGTAALGCLVKDYFPEPVTVSWNSGALT | |
| | SGVHTFPAVLQSSGLYSLSSVVTVPSSSLGTQTYICNVNHKPS | |
| | NTKVDKKVEPKSCDKTHTCPPCPAPELLGGPSVFLFPPKPKDT | |
| | LMISRTPEVTCVVVDVSHEDPEVKFNWYVDGVEVHNAKTKP | |
| | REEQYNSTYRVVSVLTVLHQDWLNGKEYKCKVSNKALPAPE | |
| | EKTISKAKGQPREPQVYTLPPSRDELTKNQVSLSCAVKGFYPS | |
| | DIAVEWESNGQPENNYKTTPPVLDSDGSFFLVSKLTVDKSRW | |
| | QQGNVFSCSVMHEALHNHYTQKSLSLSPGK | |
| 31 | EVQLVESGGGLVQPGGSLRLSCAASGFNIKDTYIHWVRQAPG | Anti-HER2_D4_heavy |
| | KGLEWVARIYPTNGYTRYADSVKGRFTISADTSKNTAYLQM | chain_v6 |
| | NSLRAEDTAVYYCSRWGGDGFYAMDYWGQGTLVTVSSAST | |
| | KGPSVFPLAPSSKSTSGGTAALGCLVKDYFPEPVTVSWNSGA | |
| | LTSGVHTFPAVLQSSGLYSLSSVVTVPSSSLGTQTYICNVNHK | |
| | PSNTKVDKKVEPKSCDKTHTCPPCPAPEAAGGPDVFLFPPKPK | |
| | DTLMISRTPEVTCVVVDVSHEDPEVKFNWYVDGVEVHNAKT | |
| | KPREEQYNSTYRVVSVLTVLHQDWLNGKEYKCKVSNKALG | |
| | APIEKTISKAKGQPREPQVYTLPPSRDELTKNQVSLWCLVKGF | |
| | YPSDIAVEWESYGTEWSNYKTTPPVLDSDGSFFLYSKLTVSKE | |
| | EWQQGFVFSCSVMHEALHNHYTQKSLSLSPGK | |
| 32 | EVQLVESGGGLVQPGGSLRLSCAASGFNIKDTYIHWVRQAPG | Anti-HER2_D4_heavy |
| | KGLEWVARIYPTNGYTRYADSVKGRFTISADTSKNTAYLQM | chain_v7 |
| | NSLRAEDTAVYYCSRWGGDGFYAMDYWGQGTLVTVSSAST | |
| | KGPSVFPLAPSSKSTSGGTAALGCLVKDYFPEPVTVSWNSGA | |
| | LTSGVHTFPAVLQSSGLYSLSSVVTVPSSSLGTQTYICNVNHK | |
| | PSNTKVDKKVEPKSCDKTHTCPPCPAPEAAGGPDVFLFPPKPK | |
| | DTLMISRTPEVTCVVVDVSHEDPEVKFNWYVDGVEVHNAKT | |
| | KPREEQYNSTYRVVSVLTVLHQDWLNGKEYKCKVSNKALSA | |
| | PIEKTISKAKGQPREPQVYTLPPSRDELTKNQVSLWCLVKGFY | |
| | PSDIAVEWESYGTEWSNYKTTPPVLDSDGSFFLYSKLTVSKEE | |
| | WQQGFVFSCSVMHEALHNHYTQKSLSLSPGK | |
| 33 | EVQLVESGGGLVQPGGSLRLSCAASGFTFTDYTMDWVRQAP | Anti-HER2_D2_heavy |
| | GKGLEWVADVNPNSGGSIYNQRFKGRFTLSVDRSKNTLYLQ | chain_v7 |
| | MNSLRAEDTAVYYCARNLGPLFYFDYWGQGTLVTVSSASTK | |
| | GPSVFPLAPSSKSTSGGTAALGCLVKDYFPEPVTVSWNSGALT | |
| | SGVHTFPAVLQSSGLYSLSSVVTVPSSSLGTQTYICNVNHKPS | |
| | NTKVDKKVEPKSCDKTHTCPPCPAPELLGGPDVFLFPPKPKDT | |
| | LMISRTPEVTCVVVDVSHEDPEVKFNWYVDGVEVHNAKTKP | |
| | REEQYNSTYRVVSVLTVLHQDWLNGKEYKCKVSNKALPAPE | |
| | EKTISKAKGQPREPQVYTLPPSRDELTKNQVSLSCAVKGFYPS | |
| | DIAVEWESNGQPENNYKTTPPVLDSDGSFFLVSKLTVDKSRW | |
| | QQGNVFSCSVMHEALHNHYTQKSLSLSPGK | |
| 34 | EVQLVESGGGLVQPGGSLRLSCAASGFTFTDYTMDWVRQAP | Anti-HER2_D2_heavy |
| | GKGLEWVADVNPNSGGSIYNQRFKGRFTLSVDRSKNTLYLQ | chain_v8 |
| | MNSLRAEDTAVYYCARNLGPLFYFDYWGQGTLVTVSSASTK | |
| | GPSVFPLAPSSKSTSGGTAALGCLVKDYFPEPVTVSWNSGALT | |
| | SGVHTFPAVLQSSGLYSLSSVVTVPSSSLGTQTYICNVNHKPS | |
| | NTKVDKKVEPKSCDKTHTCPPCPAPELLGGPSVFLFPPKPKDT | |
| | LMISRTPEVTCVVVDVSHEDPEVKFNWYVDGVEVHNAKTKP | |
| | REEQYNSTYRVVSVLTVLHQDWLNGKEYKCKVSNKALPAPE | |
| | EKTISKAKGQPREPQVYTLPPSRDELTKNQVSLSCAVKGFYPS | |
| | DIAVEWESNGQPENNYKTTPPVLDSDGSFFLVSKLTVDKSRW | |
| | QQGNVFSCSVMHEALHNHYTQKSLSLSPGK | |
| 35 | EVQLVESGGGLVQPGGSLRLSCAASGFTFTDYTMDWVRQAP | Anti-HER2_D2_heavy |
| | GKGLEWVADVNPNSGQSIYNQRFKGRFTLSVDRSKNTLYLQ | chain_v9 |
| | MNSLRAEDTAVYYCARNLGPLFYFDYWGQGTLVTVSSASTK | |
| | GPSVFPLAPSSKSTSGGTAALGCLVKDYFPEPVTVSWNSGALT | |
| | SGVHTFPAVLQSSGLYSLSSVVTVPSSSLGTQTYICNVNHKPS | |
| | NTKVDKKVEPKSCDKTHTCPPCPAPELLGGPDVFLFPPKPKDT | |
| | LMISRTPEVTCVVVDVSHEDPEVKFNWYVDGVEVHNAKTKP | |
| | REEQYNSTYRVVSVLTVLHQDWLNGKEYKCKVSNKALPAPE | |
| | EKTISKAKGQPREPQVYTLPPSRDELTKNQVSLSCAVKGFYPS | |
| | DIAVEWESNGQPENNYKTTPPVLDSDGSFFLVSKLTVDKSRW | |
| | QQGNVFSCSVMHEALHNHYTQKSLSLSPGK | |
| 36 | EVQLVESGGGLVQPGGSLRLSCAASGFTFTDYTMDWVRQAP | Anti-HER2_D2_heavy |
| | GKGLEWVADVNPNSGQSIYNQRFKGRFTLSVDRSKNTLYLQ | chain_v10 |
| | MNSLRAEDTAVYYCARNLGPLFYFDYWGQGTLVTVSSASTK | |
| | GPSVFPLAPSSKSTSGGTAALGCLVKDYFPEPVTVSWNSGALT | |
| | SGVHTFPAVLQSSGLYSLSSVVTVPSSSLGTQTYICNVNHKPS | |
| | NTKVDKKVEPKSCDKTHTCPPCPAPELLGGPSVFLFPPKPKDT | |
| | LMISRTPEVTCVVVDVSHEDPEVKFNWYVDGVEVHNAKTKP | |
| | REEQYNSTYRVVSVLTVLHQDWLNGKEYKCKVSNKALPAPE | |
| | EKTISKAKGQPREPQVYTLPPSRDELTKNQVSLSCAVKGFYPS | |
| | DIAVEWESNGQPENNYKTTPPVLDSDGSFFLVSKLTVDKSRW | |
| | QQGNVFSCSVMHEALHNHYTQKSLSLSPGK | |
| 37 | EVQLVESGGGLVQPGGSLRLSCAASGFNIKDTYIHWVRQAPG | Anti-HER2_D4_heavy |
| | KGLEWVARIYPTNGYTRYADSVKGRFTISADTSKNTAYLQM | chain_v8 |
| | NSLRAEDTAVYYCSRWGGDGFYAMDYWGQGTLVTVSSAST | |
| | KGPSVFPLAPSSKSTSGGTAALGCLVKDYFPEPVTVSWNSGA | |
| | LTSGVHTFPAVLQSSGLYSLSSVVTVPSSSLGTQTYICNVNHK | |
| | PSNTKVDKKVEPKSCDKTHTCPPCPAPEAAGGPSVFLFPPKPK | |
| | DTLMISRTPEVTCVVVDVSHEDPEVKFNWYVDGVEVHNAKT | |
| | KPREEQYNSTYRVVSVLTVLHQDWLNGKEYKCKVSNKALPA | |
| | PIEKTISKAKGQPREPQVYTLPPSRDELTKNQVSLWCLVKGFY | |
| | PSDIAVEWESYGTEWSNYKTTPPVLDSDGSFFLYSKLTVSKEE | |
| | WQQGFVFSCSVMHEALHNHYTQKSLSLSPGK | |
| 38 | EVQLVESGGGLVQPGGSLRLSCAASGFNIKDTYIHWVRQAPG | Anti-HER2_D4_heavy |
| | KGLEWVARIYPTNGYTRYADSVKGRFTISADTSKNTAYLQM | chain_v9 |
| | NSLRAEDTAVYYCSRWGGDGFYAMDYWGQGTLVTVSSAST | |
| | KGPSVFPLAPSSKSTSGGTAALGCLVKDYFPEPVTVSWNSGA | |
| | LTSGVHTFPAVLQSSGLYSLSSVVTVPSSSLGTQTYICNVNHK | |
| | PSNTKVDKKVEPKSCDKTHTCPPCPAPEAAGGPSVFLFPPKPK | |
| | DTLMISRTPEVTCVVVDVSHEDPEVKFNWYVDGVEVHNAKT | |
| | KPREEQYNSTYRVVSVLTVLHQDWLNGKEYKCKVSNKALG | |
| | APIEKTISKAKGQPREPQVYTLPPSRDELTKNQVSLWCLVKGF | |
| | YPSDIAVEWESYGTEWSNYKTTPPVLDSDGSFFLYSKLTVSKE | |
| | EWQQGFVFSCSVMHEALHNHYTQKSLSLSPGK | |
| 39 | EVQLVESGGGLVQPGGSLRLSCAASGFNIKDTYIHWVRQAPG | Anti-HER2_D4_heavy |
| | KGLEWVARIYPTNGYTRYADSVKGRFTISADTSKNTAYLQM | chain_v10 |
| | NSLRAEDTAVYYCSRWGGDGFYAMDYWGQGTLVTVSSAST | |
| | KGPSVFPLAPSSKSTSGGTAALGCLVKDYFPEPVTVSWNSGA | |
| | LTSGVHTFPAVLQSSGLYSLSSVVTVPSSSLGTQTYICNVNHK | |
| | PSNTKVDKKVEPKSCDKTHTCPPCPAPEAAGGPSVFLFPPKPK | |
| | DTLMISRTPEVTCVVVDVSHEDPEVKFNWYVDGVEVHNAKT | |
| | KPREEQYNSTYRVVSVLTVLHQDWLNGKEYKCKVSNKALSA | |
| | PIEKTISKAKGQPREPQVYTLPPSRDELTKNQVSLWCLVKGFY | |
| | PSDIAVEWESYGTEWSNYKTTPPVLDSDGSFFLYSKLTVSKEE | |
| | WQQGFVFSCSVMHEALHNHYTQKSLSLSPGK | |
| 40 | EVQLVESGGGLVQPGGSLRLSCAASGFNIKDTYIHWVRQAPG | Anti-HER2_D4_heavy |
| | KGLEWVARIYPTNGYTRYADSVKGRFTISADTSKNTAYLQM | chain_v11 |
| | NSLRAEDTAVYYCSRWGGDGFYAMDYWGQGTLVTVSSAST | |
| | KGPSVFPLAPSSKSTSGGTAALGCLVKDYFPEPVTVSWNSGA | |
| | LTSGVHTFPAVLQSSGLYSLSSVVTVPSSSLGTQTYICNVNHK | |
| | PSNTKVDKKVEPKSCDKTHTCPPCPAPEAAGGPSVFLFPPKPK | |
| | DTLMISRTPEVTCVVVDVSHEDPEVKFNWYVDGVEVHNAKT | |
| | KPREEQYNSTYRVVSVLTVLHQDWLNGKEYKCKVSNKALSA | |
| | PEEKTISKAKGQPREPQVYTLPPSRDELTKNQVSLWCLVKGF | |
| | YPSDIAVEWESYGTEWSNYKTTPPVLDSDGSFFLYSKLTVSKE | |
| | EWQQGFVFSCSVMHEALHNHYTQKSLSLSPGK | |
| 41 | EVQLVESGGGLVQPGGSLRLSCAASGFNIKDTYIHWVRQAPG | Anti-HER2_D4_heavy |
| | KGLEWVARIYPTNGYTRYADSVKGRFTISADTSKNTAYLQM | chain_v12 |
| | NSLRAEDTAVYYCSRWGGDGFYAMDYWGQGTLVTVSSAST | |
| | KGPSVFPLAPSSKSTSGGTAALGCLVKDYFPEPVTVSWNSGA | |
| | LTSGVHTFPAVLQSSGLYSLSSVVTVPSSSLGTQTYICNVNHK | |
| | PSNTKVDKKVEPKSCDKTHTCPPCPAPEAAGGPDVFLFPPKPK | |
| | DTLMISRTPEVTCVVVDVSHEDPEVKFNWYVDGVEVHNAKT | |
| | KPREEQYNSTYRVVSVLTVLHQDWLNGKEYKCKVSNKALSA | |
| | PEEKTISKAKGQPREPQVYTLPPSRDELTKNQVSLWCLVKGF | |
| | YPSDIAVEWESYGTEWSNYKTTPPVLDSDGSFFLYSKLTVSKE | |
| | EWQQGFVFSCSVMHEALHNHYTQKSLSLSPGK | |
| 42 | EVQLVESGGGLVQPGGSLRLSCAASGFNIKDTYIHWVRQAPG | Anti-HER2_D4_heavy |
| | KGLEWVARIYPTNGYTRYADSVKGRFTISADTSKNTAYLQM | chain_v13 |
| | NSLRAEDTAVYYCSRWGGDGFYAMDYWGQGTLVTVSSAST | |
| | KGPSVFPLAPSSKSTSGGTAALGCLVKDYFPEPVTVSWNSGA | |
| | LTSGVHTFPAVLQSSGLYSLSSVVTVPSSSLGTQTYICNVNHK | |
| | PSNTKVDKKVEPKSCDKTHTCPPCPAPELLGGPSVFLFPPKPK | |
| | DTLMISRTPEVTCVVVDVSHEDPEVKFNWYVDGVEVHNAKT | |
| | KPREEQYNSTYRVVSVLTVLHQDWLNGKEYKCKVSNKALPA | |
| | PIEKTISKAKGQPREPQVYTLPPSRDELTKNQVSLSCAVKGFY | |
| | PSDIAVEWESNGQPENNYKTTPPVLDSDGSFFLVSKLTVDKSR | |
| | WQQGNVFSCSVMHEALHNHYTQKSLSLSPGK | |
| 43 | EVQLVESGGGLVQPGGSLRLSCAASGFTFTDYTMDWVRQAP | Anti-HER2_D2_heavy |
| | GKGLEWVADVNPNSGQSIYNQRFKGRFTLSVDRSKNTLYLQ | chain_v11 |
| | MNSLRAEDTAVYYCARNLGPSFYFDYWGQGTLVTVSSASTK | |
| | GPSVFPLAPSSKSTSGGTAALGCLVKDYFPEPVTVSWNSGALT | |
| | SGVHTFPAVLQSSGLYSLSSVVTVPSSSLGTQTYICNVNHKPS | |
| | NTKVDKKVEPKSCDKTHTCPPCPAPEAAGGPSVFLFPPKPKD | |
| | TLMISRTPEVTCVVVDVSHEDPEVKFNWYVDGVEVHNAKTK | |
| | PREEQYNSTYRVVSVLTVLHQDWLNGKEYKCKVSNKALPAP | |
| | IEKTISKAKGQPREPQVYTLPPSRDELTKNQVSLWCLVKGFYP | |
| | SDIAVEWESYGTEWSNYKTTPPVLDSDGSFFLYSKLTVSKEE | |
| | WQQGFVFSCSVMHEALHNHYTQKSLSLSPGK | |
| 44 | EVQLVESGGGLVQPGGSLRLSCAASGFTFTDYTMDWVRQAP | Anti-HER2_D2_heavy |
| | GKGLEWVADVNPNSGGSIYNQRFKGRFTLSVDRSKNTLYLQ | chain_v12 |
| | MNSLRAEDTAVYYCARNLGPLFYFDYWGQGTLVTVSSASTK | |
| | GPSVFPLAPSSKSTSGGTAALGCLVKDYFPEPVTVSWNSGALT | |
| | SGVHTFPAVLQSSGLYSLSSVVTVPSSSLGTQTYICNVNHKPS | |
| | NTKVDKKVEPKSCDKTHTCPPCPAPEAAGGPSVFLFPPKPKD | |
| | TLMISRTPEVTCVVVDVSHEDPEVKFNWYVDGVEVHNAKTK | |
| | PREEQYNSTYRVVSVLTVLHQDWLNGKEYKCKVSNKALPAP | |
| | IEKTISKAKGQPREPQVYTLPPSRDELTKNQVSLWCLVKGFYP | |
| | SDIAVEWESYGTEWSNYKTTPPVLDSDGSFFLYSKLTVSKEE | |
| | WQQGFVFSCSVMHEALHNHYTQKSLSLSPGK | |
| 45 | EVQLVESGGGLVQPGGSLRLSCAASGFTFTDYTMDWVRQAP | Anti-HER2_D2_heavy |
| | GKGLEWVADVNPNSGQSIYNQRFKGRFTLSVDRSKNTLYLQ | chain_v13 |
| | MNSLRAEDTAVYYCARNLGPLFYFDYWGQGTLVTVSSASTK | |
| | GPSVFPLAPSSKSTSGGTAALGCLVKDYFPEPVTVSWNSGALT | |
| | SGVHTFPAVLQSSGLYSLSSVVTVPSSSLGTQTYICNVNHKPS | |
| | NTKVDKKVEPKSCDKTHTCPPCPAPEAAGGPSVFLFPPKPKD | |
| | TLMISRTPEVTCVVVDVSHEDPEVKFNWYVDGVEVHNAKTK | |
| | PREEQYNSTYRVVSVLTVLHQDWLNGKEYKCKVSNKALPAP | |
| | IEKTISKAKGQPREPQVYTLPPSRDELTKNQVSLWCLVKGFYP | |
| | SDIAVEWESYGTEWSNYKTTPPVLDSDGSFFLYSKLTVSKEE | |
| | WQQGFVFSCSVMHEALHNHYTQKSLSLSPGK | |
| 46 | EVQLVESGGGLVQPGGSLRLSCAASGFTFTDYTMDWVRQAP | Anti-HER2_D2_heavy |
| | GKGLEWVADVNPNSGQSIYNQRFKGRFTLSVDRSKNTLYLQ | chain_v14 |
| | MNSLRAEDTAVYYCARNLGPSFYFDYWGQGTLVTVSSASTK | |
| | GPSVFPLAPSSKSTSGGTAALGCLVKDYFPEPVTVSWNSGALT | |
| | SGVHTFPAVLQSSGLYSLSSVVTVPSSSLGTQTYICNVNHKPS | |
| | NTKVDKKVEPKSCDKTHTCPPCPAPELLGGPDVFLFPPKPKDT | |
| | LMISRTPEVTCVVVDVSHEDPEVKFNWYVDGVEVHNAKTKP | |
| | REEQYNSTYRVVSVLTVLHQDWLNGKEYKCKVSNKALPAPI | |
| | EKTISKAKGQPREPQVYTLPPSRDELTKNQVSLSCAVKGFYPS | |
| | DIAVEWESNGQPENNYKTTPPVLDSDGSFFLVSKLTVDKSRW | |
| | QQGNVFSCSVMHEALHNHYTQKSLSLSPGK | |
| 47 | EVQLVESGGGLVQPGGSLRLSCAASGFTFTDYTMDWVRQAP | Anti-HER2_D2_heavy |
| | GKGLEWVADVNPNSGGSIYNQRFKGRFTLSVDRSKNTLYLQ | chain_v15 |
| | MNSLRAEDTAVYYCARNLGPLFYFDYWGQGTLVTVSSASTK | |
| | GPSVFPLAPSSKSTSGGTAALGCLVKDYFPEPVTVSWNSGALT | |
| | SGVHTFPAVLQSSGLYSLSSVVTVPSSSLGTQTYICNVNHKPS | |
| | NTKVDKKVEPKSCDKTHTCPPCPAPELLGGPDVFLFPPKPKDT | |
| | LMISRTPEVTCVVVDVSHEDPEVKFNWYVDGVEVHNAKTKP | |
| | REEQYNSTYRVVSVLTVLHQDWLNGKEYKCKVSNKALPAPI | |
| | EKTISKAKGQPREPQVYTLPPSRDELTKNQVSLSCAVKGFYPS | |
| | DIAVEWESNGQPENNYKTTPPVLDSDGSFFLVSKLTVDKSRW | |
| | QQGNVFSCSVMHEALHNHYTQKSLSLSPGK | |
| 48 | EVQLVESGGGLVQPGGSLRLSCAASGFTFTDYTMDWVRQAP | Anti-HER2_D2_heavy |
| | GKGLEWVADVNPNSGQSIYNQRFKGRFTLSVDRSKNTLYLQ | chain_v16 |
| | MNSLRAEDTAVYYCARNLGPLFYFDYWGQGTLVTVSSASTK | |
| | GPSVFPLAPSSKSTSGGTAALGCLVKDYFPEPVTVSWNSGALT | |
| | SGVHTFPAVLQSSGLYSLSSVVTVPSSSLGTQTYICNVNHKPS | |
| | NTKVDKKVEPKSCDKTHTCPPCPAPELLGGPDVFLFPPKPKDT | |
| | LMISRTPEVTCVVVDVSHEDPEVKFNWYVDGVEVHNAKTKP | |
| | REEQYNSTYRVVSVLTVLHQDWLNGKEYKCKVSNKALPAPI | |
| | EKTISKAKGQPREPQVYTLPPSRDELTKNQVSLSCAVKGFYPS | |
| | DIAVEWESNGQPENNYKTTPPVLDSDGSFFLVSKLTVDKSRW | |
| | QQGNVFSCSVMHEALHNHYTQKSLSLSPGK | |
| 49 | GFNFTDYTMD | Anti-HER2_CDR-H1.1 |
| 50 | GFKFTDYTMD | Anti-HER2_CDR-H1.2 |
| 51 | GFMFTDYTMD | Anti-HER2_CDR-H1.3 |
| 52 | GFHFTDYTMD | Anti-HER2_CDR-H1.4 |
| 53 | DVNPNSGGSIYNRRFKG | Anti-HER2_CDR-H2.2 |
| 54 | DVNPNSGGSIYNHRFKG | Anti-HER2_CDR-H2.3 |
| 55 | DVNPNSGGSIYNTRFKG | Anti-HER2_CDR-H2.4 |
| 56 | ARNLGPWFYFDY | Anti-HER2_CDR-H3.2 |
| 57 | ARNLGPFFYFDY | Anti-HER2_CDR-H3.3 |
| 58 | ARNLGPDFYFDY | Anti-HER2_CDR-H3.4 |
| 59 | ARNLGPYFYFDY | Anti-HER2_CDR-H3.5 |
| 60 | EVQLVESGGGLVQPGGSLRLSCAASGFNFTDYTMDWVRQAP | Anti-HER2_D2_VH_v4 |
| | GKGLEWVADVNPNSGGSIYNQRFKGRFTLSVDRSKNTLYLQ | |
| | MNSLRAEDTAVYYCARNLGPSFYFDYWGQGTLVTVSS | |
| 61 | EVQLVESGGGLVQPGGSLRLSCAASGFKFTDYTMDWVRQAP | Anti-HER2_D2_VH_v5 |
| | GKGLEWVADVNPNSGGSIYNQRFKGRFTLSVDRSKNTLYLQ | |
| | MNSLRAEDTAVYYCARNLGPSFYFDYWGQGTLVTVSS | |
| 62 | EVQLVESGGGLVQPGGSLRLSCAASGFMFTDYTMDWVRQAP | Anti-HER2_D2_VH_v6 |
| | GKGLEWVADVNPNSGGSIYNQRFKGRFTLSVDRSKNTLYLQ | |
| | MNSLRAEDTAVYYCARNLGPSFYFDYWGQGTLVTVSS | |
| 63 | EVQLVESGGGLVQPGGSLRLSCAASGFHFTDYTMDWVRQAP | Anti-HER2_D2_VH_v7 |
| | GKGLEWVADVNPNSGGSIYNQRFKGRFTLSVDRSKNTLYLQ | |
| | MNSLRAEDTAVYYCARNLGPSFYFDYWGQGTLVTVSS | |
| 64 | EVQLVESGGGLVQPGGSLRLSCAASGFTFTDYTMDWVRQAP | Anti-HER2_D2_VH_v8 |
| | GKGLEWVADVNPNSGGSIYNRRFKGRFTLSVDRSKNTLYLQ | |
| | MNSLRAEDTAVYYCARNLGPSFYFDYWGQGTLVTVSS | |
| 65 | EVQLVESGGGLVQPGGSLRLSCAASGFTFTDYTMDWVRQAP | Anti-HER2_D2_VH_v9 |
| | GKGLEWVADVNPNSGGSIYNHRFKGRFTLSVDRSKNTLYLQ | |
| | MNSLRAEDTAVYYCARNLGPSFYFDYWGQGTLVTVSS | |
| 66 | EVQLVESGGGLVQPGGSLRLSCAASGFTFTDYTMDWVRQAP | Anti-HER2_D2_VH_v10 |
| | GKGLEWVADVNPNSGGSIYNTRFKGRFTLSVDRSKNTLYLQ | |
| | MNSLRAEDTAVYYCARNLGPSFYFDYWGQGTLVTVSS | |
| 67 | EVQLVESGGGLVQPGGSLRLSCAASGFTFTDYTMDWVRQAP | Anti-HER2_D2_VH_v11 |
| | GKGLEWVADVNPNSGGSIYNQRFKGRFTLSVDRSKNTLYLQ | |
| | MNSLRAEDTAVYYCARNLGPWFYFDYWGQGTLVTVSS | |
| 68 | EVQLVESGGGLVQPGGSLRLSCAASGFTFTDYTMDWVRQAP | Anti-HER2_D2_VH_v12 |
| | GKGLEWVADVNPNSGGSIYNQRFKGRFTLSVDRSKNTLYLQ | |
| | MNSLRAEDTAVYYCARNLGPFFYFDYWGQGTLVTVSS | |
| 69 | EVQLVESGGGLVQPGGSLRLSCAASGFTFTDYTMDWVRQAP | Anti-HER2_D2_VH_v13 |
| | GKGLEWVADVNPNSGGSIYNQRFKGRFTLSVDRSKNTLYLQ | |
| | MNSLRAEDTAVYYCARNLGPDFYFDYWGQGTLVTVSS | |
| 70 | EVQLVESGGGLVQPGGSLRLSCAASGFTFTDYTMDWVRQAP | Anti-HER2_D2_VH_v4 |
| | GKGLEWVADVNPNSGGSIYNQRFKGRFTLSVDRSKNTLYLQ | |
| | MNSLRAEDTAVYYCARNLGPYFYFDYWGQGTLVTVSS | |
| 71 | APELLGGPDVFLFPPKPKDTLMISRTPEVTCVVVDVSHEDPEV | Fc sequence with hole |
| | KFNWYVDGVEVHNAKTKPREEQYNSTYRVVSVLTVLHQDW | mutations.hD |
| | LNGKEYKCKVSNKALPAPIEKTISKAKGQPREPQVYTLPPSRD | |
| | ELTKNQVSLSCAVKGFYPSDIAVEWESNGQPENNYKTTPPVL | |
| | DSDGSFFLVSKLTVDKSRWQQGNVFSCSVMHEALHNHYTQK | |
| | SLSLSPGK | |
| 72 | APELLGGPSVFLFPPKPKDTLMISRTPEVTCVVVDVSHEDPEV | Fc sequence with hole |
| | KFNWYVDGVEVHNAKTKPREEQYNSTYRVVSVLTVLHQDW | mutations.hE |
| | LNGKEYKCKVSNKALPAPEEKTISKAKGQPREPQVYTLPPSR | |
| | DELTKNQVSLSCAVKGFYPSDIAVEWESNGQPENNYKTTPPV | |
| | LDSDGSFFLVSKLTVDKSRWQQGNVFSCSVMHEALHNHYTQ | |
| | KSLSLSPGK | |
| 73 | APELLGGPDVFLFPPKPKDTLMISRTPEVTCVVVDVSHEDPEV | Fc sequence with hole |
| | KFNWYVDGVEVHNAKTKPREEQYNSTYRVVSVLTVLHQDW | mutations.hDE |
| | LNGKEYKCKVSNKALPAPEEKTISKAKGQPREPQVYTLPPSR | |
| | DELTKNQVSLSCAVKGFYPSDIAVEWESNGQPENNYKTTPPV | |
| | LDSDGSFFLVSKLTVDKSRWQQGNVFSCSVMHEALHNHYTQ | |
| | KSLSLSPGK | |
| 74 | APEAAGGPDVFLFPPKPKDTLMISRTPEVTCVVVDVSHEDPEV | Clone CH3C.35.23.4 with |
| | KFNWYVDGVEVHNAKTKPREEQYNSTYRVVSVLTVLHQDW | knob, LALA, and kD |
| | LNGKEYKCKVSNKALPAPIEKTISKAKGQPREPQVYTLPPSRD | |
| | ELTKNQVSLWCLVKGFYPSDIAVEWESYGTEWSNYKTTPPVL | |
| | DSDGSFFLYSKLTVSKEEWQQGFVFSCSVMHEALHNHYTQK | |
| | SLSLSPGK | |
| 75 | APEAAGGPSVFLFPPKPKDTLMISRTPEVTCVVVDVSHEDPEV | Clone CH3C.35.23.4 with |
| | KFNWYVDGVEVHNAKTKPREEQYNSTYRVVSVLTVLHQDW | knob, LALA, and kE |
| | LNGKEYKCKVSNKALPAPEEKTISKAKGQPREPQVYTLPPSR | |
| | DELTKNQVSLWCLVKGFYPSDIAVEWESYGTEWSNYKTTPP | |
| | VLDSDGSFFLYSKLTVSKEEWQQGFVFSCSVMHEALHNHYT | |
| | QKSLSLSPGK | |
| 76 | APEAAGGPDVFLFPPKPKDTLMISRTPEVTCVVVDVSHEDPEV | Clone CH3C.35.23.4 with |
| | KFNWYVDGVEVHNAKTKPREEQYNSTYRVVSVLTVLHQDW | knob, LALA, and kDE |
| | LNGKEYKCKVSNKALPAPEEKTISKAKGQPREPQVYTLPPSR | |
| | DELTKNQVSLWCLVKGFYPSDIAVEWESYGTEWSNYKTTPP | |
| | VLDSDGSFFLYSKLTVSKEEWQQGFVFSCSVMHEALHNHYT | |
| | QKSLSLSPGK | |
| 77 | APEAAGGPSVFLFPPKPKDTLMISRTPEVTCVVVDVSHEDPEV | Clone CH3C.35.23.4 with |
| | KFNWYVDGVEVHNAKTKPREEQYNSTYRVVSVLTVLHQDW | knob, LALA, and PG |
| | LNGKEYKCKVSNKALGAPIEKTISKAKGQPREPQVYTLPPSRD | |
| | ELTKNQVSLWCLVKGFYPSDIAVEWESYGTEWSNYKTTPPVL | |
| | DSDGSFFLYSKLTVSKEEWQQGFVFSCSVMHEALHNHYTQK | |
| | SLSLSPGK | |
| 78 | APEAAGGPSVFLFPPKPKDTLMISRTPEVTCVVVDVSHEDPEV | Clone CH3C.35.23.4 with |
| | KFNWYVDGVEVHNAKTKPREEQYNSTYRVVSVLTVLHQDW | knob, LALA, and PS |
| | LNGKEYKCKVSNKALSAPIEKTISKAKGQPREPQVYTLPPSRD | |
| | ELTKNQVSLWCLVKGFYPSDIAVEWESYGTEWSNYKTTPPVL | |
| | DSDGSFFLYSKLTVSKEEWQQGFVFSCSVMHEALHNHYTQK | |
| | SLSLSPGK | |
| 79 | APEAAGGPDVFLFPPKPKDTLMISRTPEVTCVVVDVSHEDPEV | Clone CH3C.35.23.4 with |
| | KFNWYVDGVEVHNAKTKPREEQYNSTYRVVSVLTVLHQDW | knob, LALA, PG, and kD |
| | LNGKEYKCKVSNKALGAPIEKTISKAKGQPREPQVYTLPPSRD | |
| | ELTKNQVSLWCLVKGFYPSDIAVEWESYGTEWSNYKTTPPVL | |
| | DSDGSFFLYSKLTVSKEEWQQGFVFSCSVMHEALHNHYTQK | |
| | SLSLSPGK | |
| 80 | APEAAGGPDVFLFPPKPKDTLMISRTPEVTCVVVDVSHEDPEV | Clone CH3C.35.23.4 with |
| | KFNWYVDGVEVHNAKTKPREEQYNSTYRVVSVLTVLHQDW | knob, LALA, PS, and kD |
| | LNGKEYKCKVSNKALSAPIEKTISKAKGQPREPQVYTLPPSRD | |
| | ELTKNQVSLWCLVKGFYPSDIAVEWESYGTEWSNYKTTPPVL | |
| | DSDGSFFLYSKLTVSKEEWQQGFVFSCSVMHEALHNHYTQ | |
| | KSLSLSPGK | |
| 81 | APEAAGGPSVFLFPPKPKDTLMISRTPEVTCVVVDVSHEDPEV | Clone CH3C.35.23.4 with |
| | KFNWYVDGVEVHNAKTKPREEQYNSTYRVVSVLTVLHQDW | knob, LALA, PG, and kE |
| | LNGKEYKCKVSNKALGAPEEKTISKAKGQPREPQVYTLPPSR | |
| | DELTKNQVSLWCLVKGFYPSDIAVEWESYGTEWSNYKTTPP | |
| | VLDSDGSFFLYSKLTVSKEEWQQGFVFSCSVMHEALHNHYT | |
| | QKSLSLSPGK | |
| 82 | APEAAGGPSVFLFPPKPKDTLMISRTPEVTCVVVDVSHEDPEV | Clone CH3C.35.23.4 with |
| | KFNWYVDGVEVHNAKTKPREEQYNSTYRVVSVLTVLHQDW | knob, LALA, PS, and kE |
| | LNGKEYKCKVSNKALSAPEEKTISKAKGQPREPQVYTLPPSR | |
| | DELTKNQVSLWCLVKGFYPSDIAVEWESYGTEWSNYKTTPP | |
| | VLDSDGSFFLYSKLTVSKEEWQQGFVFSCSVMHEALHNHYT | |
| | QKSLSLSPGK | |
| 83 | APEAAGGPDVFLFPPKPKDTLMISRTPEVTCVVVDVSHEDPEV | Clone CH3C.35.23.4 with |
| | KFNWYVDGVEVHNAKTKPREEQYNSTYRVVSVLTVLHQDW | knob, LALA, PG, and kDE |
| | LNGKEYKCKVSNKALGAPEEKTISKAKGQPREPQVYTLPPSR | |
| | DELTKNQVSLWCLVKGFYPSDIAVEWESYGTEWSNYKTTPP | |
| | VLDSDGSFFLYSKLTVSKEEWQQGFVFSCSVMHEALHNHYT | |
| | QKSLSLSPGK | |
| 84 | APEAAGGPDVFLFPPKPKDTLMISRTPEVTCVVVDVSHEDPEV | Clone CH3C.35.23.4 with |
| | KFNWYVDGVEVHNAKTKPREEQYNSTYRVVSVLTVLHQDW | knob, LALA, PS, and kDE |
| | LNGKEYKCKVSNKALSAPEEKTISKAKGQPREPQVYTLPPSR | |
| | DELTKNQVSLWCLVKGFYPSDIAVEWESYGTEWSNYKTTPP | |
| | VLDSDGSFFLYSKLTVSKEEWQQGFVFSCSVMHEALHNHYT | |
| | QKSLSLSPGK | |
| 85 | APELLGGPSVFLFPPKPKDTLMISRTPEVTCVVVDVSHEDPEV | Fc sequence with hole |
| | KFNWYVDGVEVHNAKTKPREEQYNSTYRVVSVLTVLHQDW | mutations |
| | LNGKEYKCKVSNKALPAPIEKTISKAKGQPREPQVYTLPPSRD | |
| | ELTKNQVSLSCAVKGFYPSDIAVEWESNGQPENNYKTTPPVL | |
| | DSDGSFFLVSKLTVDKSRWQQGNVFSCSVMHEALHNHYTQK | |
| | SLSLSPGK | |
| 86 | APEAAGGPSVFLFPPKPKDTLMISRTPEVTCVVVDVSHEDPEV | Clone CH3C.35.23.4 with |
| | KFNWYVDGVEVHNAKTKPREEQYNSTYRVVSVLTVLHQDW | knob and LALA mutations |
| | LNGKEYKCKVSNKALPAPIEKTISKAKGQPREPQVYTLPPSRD | |
| | ELTKNQVSLWCLVKGFYPSDIAVEWESYGTEWSNYKTTPPVL | |
| | DSDGSFFLYSKLTVSKEEWQQGFVFSCSVMHEALHNHYTQK | |
| | SLSLSPGK | |
| 87 | DIQMTQSPSSLSASVGDRVTITCRASQDVNTAVAWYQQKPGK | Anti-HER2_D4_light |
| | APKLLIYSASFLYSGVPSRFSGSRSGTDFTLTISSLQPEDFATYY | chain_control |
| | CQQHYTTPPTFGQGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| 88 | DIQMTQSPSSLSASVGDRVTITCRASQDVNTAVAWYQQKPGK | Anti-HER2_light |
| | APKLLIYSASFLYSGVPSRFSGSRSGTDFTLTISSLQPEDFATYY | chain_consensus |
| | CQQXYTTPPTFGQGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| 89 | GFX₁X₂X₃DYTMD | Anti- |
| | | HER2_CDR1_consensus |
| 90 | DVX₁PX₂X₃X₄X₅SIYNX₆RFKG | Anti- |
| | | HER2_CDR2_consensus |
| 91 | ARNX₁X₂X₃X₄FYFDY | Anti- |
| | | HER2_CDR3_consensus |
| 92 | EVQLVESGGGLVQPGGSLRLSCAASGFTFTDYTMDWVRQAP | Anti-HER2_D2_heavy |
| | GKGLEWVADVNPNSGGSIYNQRFKGRFTLSVDRSKNTLYLQ | chain_control |
| | MNSLRAEDTAVYYCARNLGPSFYFDYWGQGTLVTVSSASTK | |
| | GPSVFPLAPSSKSTSGGTAALGCLVKDYFPEPVTVSWNSGALT | |
| | SGVHTFPAVLQSSGLYSLSSVVTVPSSSLGTQTYICNVNHKPS | |
| | NTKVDKKVEPKSCDKTHTCPPCPAPELLGGPSVFLFPPKPKDT | |
| | LMISRTPEVTCVVVDVSHEDPEVKFNWYVDGVEVHNAKTKP | |
| | REEQYNSTYRVVSVLTVLHQDWLNGKEYKCKVSNKALPAPI | |
| | EKTISKAKGQPREPQVYTLPPSREEMTKNQVSLTCLVKGFYPS | |
| | DIAVEWESNGQPENNYKTTPPVLDSDGSFFLYSKLTVDKSRW | |
| | QQGNVFSCSVMHEALHNHYTQKSLSLSPG | |
| 93 | EVQLVESGGGLVQPGGSLRLSCAASGFNIKDTYIHWVRQAPG | Anti-HER2_D4_heavy |
| | KGLEWVARIYPTNGYTRYADSVKGRFTISADTSKNTAYLQM | chain_control |
| | NSLRAEDTAVYYCSRWGGDGFYAMDYWGQGTLVTVSSAST | |
| | KGPSVFPLAPSSKSTSGGTAALGCLVKDYFPEPVTVSWNSGA | |
| | LTSGVHTFPAVLQSSGLYSLSSVVTVPSSSLGTQTYICNVNHK | |
| | PSNTKVDKKVEPKSCDKTHTCPPCPAPELLGGPSVFLFPPKPK | |
| | DTLMISRTPEVTCVVVDVSHEDPEVKFNWYVDGVEVHNAKT | |
| | KPREEQYNSTYRVVSVLTVLHQDWLNGKEYKCKVSNKALPA | |
| | PIEKTISKAKGQPREPQVYTLPPSREEMTKNQVSLTCLVKGFY | |
| | PSDIAVEWESNGQPENNYKTTPPVLDSDGSFFLYSKLTVDKSR | |
| | WQQGNVFSCSVMHEALHNHYTQKSLSLSPG | |
| 94 | DIQMTQSPSSLSASVGDRVTITCKASQDVSIGVAWYQQKPGK | Anti-HER2_D2_light |
| | APKLLIYSASYRYTGVPSRFSGSGSGTDFTLTISSLQPEDFATY | chain_control |
| | YCQQYYIYPYTFGQGTKVEIKRTVAAPSVFIFPPSDEQLKSGT | |
| | ASVVCLLNNFYPREAKVQWKVDNALQSGNSQESVTEQDSKD | |
| | STYSLSSTLTLSKADYEKHKVYACEVTHQGLSSPVTKSFNRG | |
| | EC | |
| 95 | APELLGGPSVFLFPPKPKDTLMISRTPEVTCVVVDVSHEDPEV | Wild-type human Fc |
| | KFNWYVDGVEVHNAKTKPREEQYNSTYRVVSVLTVLHQDW | sequence |
| | LNGKEYKCKVSNKALPAPIEKTISKAKGQPREPQVYTLPPSRD | |
| | ELTKNQVSLTCLVKGFYPSDIAVEWESNGQPENNYKTTPPVL | |
| | DSDGSFFLYSKLTVDKSRWQQGNVFSCSVMHEALHNHYTQK | |
| | SLSLSPGK | |
| 96 | EPKSCDKTHTCPPCP | Human IgG1 hinge region |
| 97 | APELLGGPSVFLFPPKPKDTLMISRTPEVTCVVVDVSHEDPEV | Clone CH3C.35.23.4 |
| | KFNWYVDGVEVHNAKTKPREEQYNSTYRVVSVLTVLHQDW | |
| | LNGKEYKCKVSNKALPAPIEKTISKAKGQPREPQVYTLPPSRD | |
| | ELTKNQVSLTCLVKGFYPSDIAVEWESYGTEWSNYKTTPPVL | |
| | DSDGSFFLYSKLTVSKEEWQQGFVFSCSVMHEALHNHYTQK | |
| | SLSLSPGK | |
| 98 | APELLGGPSVFLFPPKPKDTLMISRTPEVTCVVVDVSHEDPEV | Clone CH3C.35.23.4 with |
| | KFNWYVDGVEVHNAKTKPREEQYNSTYRVVSVLTVLHQDW | knob mutation |
| | LNGKEYKCKVSNKALPAPIEKTISKAKGQPREPQVYTLPPSRD | |
| | ELTKNQVSLWCLVKGFYPSDIAVEWESYGTEWSNYKTTPPVL | |
| | DSDGSFFLYSKLTVSKEEWQQGFVFSCSVMHEALHNHYTQK | |
| | SLSLSPGK | |
| 99 | APELLGGPSVFLFPPKPKDTLMISRTPEVTCVVVDVSHEDPEV | Clone CH3C.35.23.4 with |
| | KFNWYVDGVEVHNAKTKPREEQYNSTYRVVSVLTVLHQDW | hole mutations |
| | LNGKEYKCKVSNKALPAPIEKTISKAKGQPREPQVYTLPPSRD | |
| | ELTKNQVSLSCAVKGFYPSDIAVEWESYGTEWSNYKTTPPVL | |
| | DSDGSFFLVSKLTVSKEEWQQGFVFSCSVMHEALHNHYTQK | |
| | SLSLSPGK | |
| 100 | APEAAGGPSVFLFPPKPKDTLMISRTPEVTCVVVDVSHEDPEV | Clone CH3C.35.23.4 with |
| | KFNWYVDGVEVHNAKTKPREEQYNSTYRVVSVLTVLHQDW | hole and LALA mutations |
| | LNGKEYKCKVSNKALPAPIEKTISKAKGQPREPQVYTLPPSRD | |
| | ELTKNQVSLSCAVKGFYPSDIAVEWESYGTEWSNYKTTPPVL | |
| | DSDGSFFLVSKLTVSKEEWQQGFVFSCSVMHEALHNHYTQK | |
| | SLSLSPGK | |
| 101 | APELLGGPSVFLFPPKPKDTLMISRTPEVTCVVVDVSHEDPEV | Clone CH3C.35.23.2 |
| | KFNWYVDGVEVHNAKTKPREEQYNSTYRVVSVLTVLHQDW | |
| | LNGKEYKCKVSNKALPAPIEKTISKAKGQPREPQVYTLPPSRD | |
| | ELTKNQVSLTCLVKGFYPSDIAVEWESYGTEWANYKTTPPVL | |
| | DSDGSFFLYSKLTVTKEEWQQGFVFSCSVMHEALHNHYTQK | |
| | SLSLSPGK | |
| 102 | APELLGGPSVFLFPPKPKDTLMISRTPEVTCVVVDVSHEDPEV | Clone CH3C.35.23.2 with |
| | KFNWYVDGVEVHNAKTKPREEQYNSTYRVVSVLTVLHQDW | knob mutation |
| | LNGKEYKCKVSNKALPAPIEKTISKAKGQPREPQVYTLPPSRD | |
| | ELTKNQVSLWCLVKGFYPSDIAVEWESYGTEWANYKTTPPV | |
| | LDSDGSFFLYSKLTVTKEEWQQGFVFSCSVMHEALHNHYTQ | |
| | KSLSLSPGK | |
| 103 | APEAAGGPSVFLFPPKPKDTLMISRTPEVTCVVVDVSHEDPEV | Clone CH3C.35.23.2 with |
| | KFNWYVDGVEVHNAKTKPREEQYNSTYRVVSVLTVLHQDW | knob and LALA mutations |
| | LNGKEYKCKVSNKALPAPIEKTISKAKGQPREPQVYTLPPSRD | |
| | ELTKNQVSLWCLVKGFYPSDIAVEWESYGTEWANYKTTPPV | |
| | LDSDGSFFLYSKLTVTKEEWQQGFVFSCSVMHEALHNHYTQ | |
| | KSLSLSPGK | |
| 104 | APELLGGPSVFLFPPKPKDTLMISRTPEVTCVVVDVSHEDPEV | Clone CH3C.35.23.2 with |
| | KFNWYVDGVEVHNAKTKPREEQYNSTYRVVSVLTVLHQDW | hole mutations |
| | LNGKEYKCKVSNKALPAPIEKTISKAKGQPREPQVYTLPPSRD | |
| | ELTKNQVSLSCAVKGFYPSDIAVEWESYGTEWANYKTTPPVL | |
| | DSDGSFFLVSKLTVTKEEWQQGFVFSCSVMHEALHNHYTQK | |
| | SLSLSPGK | |
| 105 | APEAAGGPSVFLFPPKPKDTLMISRTPEVTCVVVDVSHEDPEV | Clone CH3C.35.23.2 with |
| | KFNWYVDGVEVHNAKTKPREEQYNSTYRVVSVLTVLHQDW | hole and LALA mutations |
| | LNGKEYKCKVSNKALPAPIEKTISKAKGQPREPQVYTLPPSRD | |
| | ELTKNQVSLSCAVKGFYPSDIAVEWESYGTEWANYKTTPPVL | |
| | DSDGSFFLVSKLTVTKEEWQQGFVFSCSVMHEALHNHYTQK | |
| | SLSLSPGK | |
| 106 | APELLGGPSVFLFPPKPKDTLMISRTPEVTCVVVDVSHEDPEV | Clone CH3C.35.20.1 |
| | KFNWYVDGVEVHNAKTKPREEQYNSTYRVVSVLTVLHQDW | |
| | LNGKEYKCKVSNKALPAPIEKTISKAKGQPREPQVYTLPPSRD | |
| | ELTKNQVSLTCLVKGFYPSDIAVEWESFGTEWSSYKTTPPVL | |
| | DSDGSFFLYSKLTVTKEEWQQGFVFSCSVMHEALHNHYTQK | |
| | SLSLSPGK | |
| 107 | APELLGGPSVFLFPPKPKDTLMISRTPEVTCVVVDVSHEDPEV | Fc sequence with knob |
| | KFNWYVDGVEVHNAKTKPREEQYNSTYRVVSVLTVLHQDW | mutation |
| | LNGKEYKCKVSNKALPAPIEKTISKAKGQPREPQVYTLPPSRD | |
| | ELTKNQVSLWCLVKGFYPSDIAVEWESNGQPENNYKTTPPVL | |
| | DSDGSFFLYSKLTVDKSRWQQGNVFSCSVMHEALHNHYTQK | |
| | SLSLSPGK | |
| 108 | EVQLVESGGGLVQPGGSLRLSCAASGFNIKDTYIHWVRQAPG | Anti-HER2_D4_VH_v1 |
| | KGLEWVARIYPTNGYTRYADSVKGRFTISADTSKNTAYLQM | |
| | NSLRAEDTAVYYCSRWGGEGFYAMDYWGQGTLVTVSS | |
| 109 | EVQLVESGGGLVQPGGSLRLSCAASGFTFNDYTMDWVRQAP | Anti-HER2_D2_VH_v4 |
| | GKGLEWVADVNPNSGGSIVNRRFKGRFTLSVDRSKNTLYLQ | |
| | MNSLRAEDTAVYYCARNLGPFFYFDYWGQGTLVTVSS | |

1. An isolated antibody comprising:
(a) a first heavy chain comprising a first heavy chain CDR1 comprising the amino acid sequence of SEQ ID NO:16, a first heavy chain CDR2 comprising the amino acid sequence of SEQ ID NO:17, and a first heavy chain CDR3 comprising the amino acid sequence of SEQ ID NO:18;
(b) a second heavy chain comprises a second heavy chain CDR1 comprising the amino acid sequence of SEQ ID NO:4; a second heavy chain CDR2 comprising the amino acid sequence of SEQ ID NO:5 or 6, and a second heavy chain CDR3 comprising the amino acid sequence of SEQ ID NO:7 or 8;
(c) first and second light chains, wherein the first and second light chains each comprise a light chain CDR1 comprising the amino acid sequence of SEQ ID NO:11, a light chain CDR2 comprising the amino acid sequence of SEQ ID NO:12, and a light chain CDR2 comprising the amino acid sequence of SEQ ID NO:13 or 14;
(d) a first Fc polypeptide comprising a modified CH3 domain that contains modifications that create a TfR-binding site and one or more modifications that reduce TfR-mediated effector function, wherein a first Fc polypeptide comprises:
(i) Glu, Leu, Ser, Val, Trp, Tyr, or Gln at position 380;
Leu, Tyr, Phe, Trp, Met, Pro, or Val at position 384;
Leu, Thr, His, Pro, Asn, Val, or Phe at position 386;
Val, Pro, Ile, or an acidic amino acid at position 387;
Trp at position 388;
an aliphatic amino acid, Gly, Ser, Thr, or Asn at position 389;
Gly, His, Gln, Leu, Lys, Val, Phe, Ser, Ala, Asp, Glu, Asn, Arg, or Thr at position 390;
an acidic amino acid, Ala, Ser, Leu, Thr, Pro, Ile, or His at position 413;
Glu, Ser, Asp, Gly, Thr, Pro, Gln, or Arg at position 415;
Thr, Arg, Asn, or an acidic amino acid at position 416; and
an aromatic amino acid, His, or Lys at position 421; and
(ii) an Ala at position 234 and Ala at position 235,
wherein the positions are according to EU numbering; and
(e) a second Fc polypeptide does not contain a TfR-binding site or any modifications that reduce effector function.

2. The isolated antibody of claim 1, wherein:
(i) the second heavy chain CDR2 comprises the amino acid sequence of SEQ ID NO:5 and the second heavy chain CDR3 comprises the amino acid sequence of SEQ ID NO:7;
(ii) the second heavy chain CDR2 comprises the amino acid sequence of SEQ ID NO:5 and the second heavy chain CDR3 comprises the amino acid sequence of SEQ ID NO:8;
(iii) the second heavy chain CDR2 comprises the amino acid sequence of SEQ ID NO:6 and the second heavy chain CDR3 comprises the amino acid sequence of SEQ ID NO:7; or
(iv) the second heavy chain CDR2 comprises the amino acid sequence of SEQ ID NO:6 and the second heavy chain CDR3 comprises the amino acid sequence of SEQ ID NO:8.

3. The isolated antibody of claim 1, wherein:
(a) the first heavy chain comprises the amino acid sequence of SEQ ID NO:15, or an amino acid sequence having at least 90% sequence identity to SEQ ID NO:15;
(b) the second heavy chain comprises the amino acid sequence of SEQ ID NO:1, 2, or 3 or an amino acid sequence having at least 90% sequence identity to SEQ ID NO:1, 2, or 3; and
(c) the first and second light chains each comprises a light chain variable region comprising the amino acid sequence of SEQ ID NO:9 or 10 or an amino acid sequence having at least 90% sequence identity to SEQ ID NO:9 or 10.

4. The isolated antibody of claim 1, wherein:
(i) the first heavy chain comprises the amino acid sequence of SEQ ID NO:15, the second heavy chain comprises the amino acid sequence of SEQ ID NO:1, and the first and second light chains each comprises a light chain variable region comprising the amino acid sequence of SEQ ID NO:9;
(ii) the first heavy chain comprises the amino acid sequence of SEQ ID NO:15, the second heavy chain comprises the amino acid sequence of SEQ ID NO:2, and the first and second light chains each comprises a light chain variable region comprising the amino acid sequence of SEQ ID NO:9;
(iii) the first heavy chain comprises the amino acid sequence of SEQ ID NO:15, the second heavy chain comprises the amino acid sequence of SEQ ID NO:3, and the first and second light chains each comprises a light chain variable region comprising the amino acid sequence of SEQ ID NO:9;
(iv) the first heavy chain comprises the amino acid sequence of SEQ ID NO:15, the second heavy chain comprises the amino acid sequence of SEQ ID NO:1, and the first and second light chains each comprises a light chain variable region comprising the amino acid sequence of SEQ ID NO:10;
(v) the first heavy chain comprises the amino acid sequence of SEQ ID NO:15, the second heavy chain comprises the amino acid sequence of SEQ ID NO:2, and the first and second light chains each comprises a light chain variable region comprising the amino acid sequence of SEQ ID NO:10; or
(vi) the first heavy chain comprises the amino acid sequence of SEQ ID NO:15, the second heavy chain comprises the amino acid sequence of SEQ ID NO:3, and the first and second light chains each comprises a light chain variable region comprising the amino acid sequence of SEQ ID NO:10.

5. The isolated antibody of claim 1, wherein the modified CH3 domain comprises:
Glu at position 380;
Tyr at position 384;
Thr at position 386;
Glu at position 387;
Trp at position 388;
Val at position 389;
Asn at position 390;
Thr at position 413;
Glu or Ser at position 415;
Glu at position 416; and
Phe at position 421.

6. The isolated antibody of claim 1, wherein the first Fc polypeptide further comprises: a G at position 329 or a Ser at position 329, wherein the position is according to EU numbering.

7. The isolated antibody of claim 1, wherein the second Fc polypeptide comprises a Leu at position 234, a Leu a position 235, and a Pro at position 329, wherein the positions are according to EU numbering.

8. The isolated antibody of claim 1, wherein:
(a) the first heavy chain comprises the first Fc polypeptide and the second heavy chain comprises the second Fc polypeptide; or
(b) the first heavy chain comprises the second Fc polypeptide and the second heavy chain comprises the first Fc polypeptide.

9. The isolated antibody of claim 1, wherein an antibody hinge region or a portion thereof is linked to the N-terminus of the first Fc polypeptide and/or the second Fc polypeptide.

10. The isolated antibody of claim 1, wherein the first Fc polypeptide and/or the second Fc polypeptide further independently comprises a S239D substitution and/or an I332E substitution, wherein the positions are according to EU numbering.

11. The isolated antibody of claim 1, wherein:
(a) the first Fc polypeptide comprises a S239D substitution;
(b) the second Fc polypeptide comprises a S239D substitution;
(c) the first Fc polypeptide comprises a S239D substitution and the second Fc polypeptide comprises a S239D substitution;
(d) the first Fc polypeptide comprises an I332E substitution;
(e) the second Fc polypeptide comprises an I332E substitution;
(f) the first Fc polypeptide comprises an I332E substitution and the second Fc polypeptide comprises an I332E substitution;
(g) the first Fc polypeptide comprises a S239D substitution and the second Fc polypeptide comprises an I332E substitution;
(h) the first Fc polypeptide comprises an I332E substitution and the second Fc polypeptide comprises a S239D substitution;
(i) the first Fc polypeptide comprises a S239D substitution and an I332E substitution;
(j) the second Fc polypeptide comprises a S239D substitution and an I332E substitution;
(k) the first Fc polypeptide comprises a S239D substitution and an I332E substitution and the second Fc polypeptide comprises a S239D substitution;
(l) the first Fc polypeptide comprises a S239D substitution and an I332E substitution and the second Fc polypeptide comprises an I332E substitution;
(m) the first Fc polypeptide comprises a S239D substitution and the second Fc polypeptide comprises a S239D substitution and an I332E substitution;
(n) the first Fc polypeptide comprises an I332E substitution and the second Fc polypeptide comprises a S239D substitution and an I332E substitution; or
(o) the first Fc polypeptide comprises a S239D substitution and an I332E substitution and the second Fc polypeptide comprises a S239D substitution and an I332E substitution.

12. The isolated antibody of claim 1, wherein the first Fc polypeptide and the second Fc polypeptide each further comprise modifications that promote heterodimerization.

13. The isolated antibody of claim 12, wherein:
(a) the first Fc polypeptide comprises a T366W substitution and the second Fc polypeptide comprises a T366S substitution;
(b) the second Fc polypeptide comprises a T366W substitution and the first Fc polypeptide comprises a T366S substitution;
(c) the first Fc polypeptide comprises a T366W substitution and the second Fc polypeptide comprises T366S, L368A, and Y407V; or
(d) the second Fc polypeptide comprises a T366W substitution and the first Fc polypeptide comprises T366S, L368A, and Y407V substitutions,
wherein the positions are according to EU numbering.

14. A pharmaceutical composition comprising the isolated antibody of claim 1 and a pharmaceutically acceptable carrier and/or excipient.

15. Isolated polynucleotides or one or more expression vectors comprising nucleotide sequences encoding polypeptides that comprise the isolated antibody of claim 1.

16. A host cell comprising the nucleotide sequences of claim 15.

17. A method of treating a HER2-positive cancer in a subject, the method comprising administering to the subject a therapeutically effective amount of the pharmaceutical composition of claim 14.

18. The method of claim 17, wherein the cancer is: a breast cancer, an ovarian cancer, a bladder cancer, a salivary gland cancer, an endometrial cancer, a pancreatic cancer, a non-small-cell lung cancer, a gastric adenocarcinoma, or a gastroesophageal junction adenocarcinoma.

19. The method of claim 18, wherein the cancer is: a metastatic cancer or a brain metastasis of the cancer.

20. The method of claim 19, wherein the isolated antibody is administered in combination with a chemotherapy or radiation therapy.

21. An isolated antibody comprising:
(a) a first heavy chain comprising (i) a first heavy chain CDR1 comprising the amino acid sequence of SEQ ID NO:16, a first heavy chain CDR2 comprising the amino acid sequence of SEQ ID NO:17, and a first heavy chain CDR3 comprising the amino acid sequence of SEQ ID NO:18; (ii) a hinge region comprising SEQ ID NO:96; and (iii) a first Fc polypeptide comprising:
an Ala at position 234,
an Ala at position 235,
an Asp at position 239,
a Ser at position 329,
a Trp at position 366,
Glu, Leu, Ser, Val, Trp, Tyr, or Gln at position 380;
Leu, Tyr, Phe, Trp, Met, Pro, or Val at position 384;
Leu, Thr, His, Pro, Asn, Val, or Phe at position 386;
Val, Pro, Ile, or an acidic amino acid at position 387;
Trp at position 388;
an aliphatic amino acid, Gly, Ser, Thr, or Asn at position 389;
Gly, His, Gln, Leu, Lys, Val, Phe, Ser, Ala, Asp, Glu, Asn, Arg, or Thr at position 390;
an acidic amino acid, Ala, Ser, Leu, Thr, Pro, Ile, or His at position 413;
Glu, Ser, Asp, Gly, Thr, Pro, Gln, or Arg at position 415;
Thr, Arg, Asn, or an acidic amino acid at position 416; and
an aromatic amino acid, His, or Lys at position 421,
wherein the positions are according to EU numbering;
(b) (i) a second heavy chain comprises a second heavy chain CDR1 comprising the amino acid sequence of SEQ ID NO:4; a second heavy chain CDR2 comprising the amino acid sequence of SEQ ID NO:5 or 6, and a second heavy chain CDR3 comprising the amino acid sequence of SEQ ID NO:7 or 8; (ii) a hinge region comprising SEQ ID NO:96; and (iii) a second Fc polypeptide comprising:
an Asp at position 239,
a Ser at position 366,
an Ala at position 368, and
a Val at position 407,
wherein the positions are according to EU numbering; and
(c) first and second light chains, wherein the first and second light chains each comprise a light chain CDR1 comprising the amino acid sequence of SEQ ID NO:11, a light chain CDR2 comprising the amino acid sequence of SEQ ID NO:12, and a light chain CDR2 comprising the amino acid sequence of SEQ ID NO:13 or 14.

22. The isolated antibody of claim 21, wherein:
(a) the first heavy chain comprises the amino acid sequence of SEQ ID NO:15, or an amino acid sequence having at least 90% sequence identity to SEQ ID NO:15;
(b) the second heavy chain comprises the amino acid sequence of SEQ ID NO:1, 2, or 3 or an amino acid sequence having at least 90% sequence identity to SEQ ID NO:1, 2, or 3; and
(c) the first and second light chains each comprises a light chain variable region comprising the amino acid sequence of SEQ ID NO:9 or 10 or an amino acid sequence having at least 90% sequence identity to SEQ ID NO:9 or 10.

23. The isolated antibody of claim 22, wherein:
(i) the first heavy chain comprises the amino acid sequence of SEQ ID NO:15, the second heavy chain comprises the amino acid sequence of SEQ ID NO:1, and the first and second light chains each comprises a light chain variable region comprising the amino acid sequence of SEQ ID NO:9;
(ii) the first heavy chain comprises the amino acid sequence of SEQ ID NO:15, the second heavy chain comprises the amino acid sequence of SEQ ID NO:2, and the first and second light chains each comprises a light chain variable region comprising the amino acid sequence of SEQ ID NO:9;
(iii) the first heavy chain comprises the amino acid sequence of SEQ ID NO:15, the second heavy chain comprises the amino acid sequence of SEQ ID NO:3, and the first and second light chains each comprises a light chain variable region comprising the amino acid sequence of SEQ ID NO:9;
(iv) the first heavy chain comprises the amino acid sequence of SEQ ID NO:15, the second heavy chain comprises the amino acid sequence of SEQ ID NO:1, and the first and second light chains each comprises a light chain variable region comprising the amino acid sequence of SEQ ID NO:10;
(v) the first heavy chain comprises the amino acid sequence of SEQ ID NO:15, the second heavy chain comprises the amino acid sequence of SEQ ID NO:2, and the first and second light chains each comprises a light chain variable region comprising the amino acid sequence of SEQ ID NO:10; or
(vi) the first heavy chain comprises the amino acid sequence of SEQ ID NO:15, the second heavy chain comprises the amino acid sequence of SEQ ID NO:3, and the first and second light chains each comprises a light chain variable region comprising the amino acid sequence of SEQ ID NO:10.

24. A pharmaceutical composition comprising the isolated antibody of claim 21 and a pharmaceutically acceptable carrier and/or excipient.

25. Isolated polynucleotides or one or more expression vectors comprising nucleotide sequences encoding polypeptides that comprise the isolated antibody of claim 21.

26. A host cell comprising the nucleotide sequences of claim 25.

27. A method of treating a HER2-positive cancer in a subject, the method comprising administering to the subject a therapeutically effective amount of the pharmaceutical composition of claim 24.

28. The method of claim 27, wherein the cancer is: a breast cancer, an ovarian cancer, a bladder cancer, a salivary gland cancer, an endometrial cancer, a pancreatic cancer, a non-small-cell lung cancer, a gastric adenocarcinoma, or a gastroesophageal junction adenocarcinoma.

29. The method of claim 28, wherein the cancer is: a metastatic cancer or a brain metastasis of the cancer.

30. The method of claim 27, wherein the isolated antibody is administered in combination with a chemotherapy or radiation therapy.
